(19) United States
(12) Patent Application Publication
(10) Pub. No.: US 2023/0110188 A1
(43) Pub. Date: Apr. 13, 2023

(54) MuSK AGONIST ANTIBODY

(71) Applicants: argenx IIP BV, Ghent (BE); ACADEMISCH ZIEKENHUIS LEIDEN, Leiden (NL)

(72) Inventors: Christophe BLANCHETOT, Ghent (BE); Christophe STEYAERT, Ghent (BE); Dana VERGOOSSEN, Leiden (NL); Jakob Jan PLOMP, Leiden (NL); Kasra SAMAREH TALEBI, Leiden (NL); Johannes Justus Gerard Maria VERSCHUUREN, Leiden (NL); Silvère Maria VAN DER MAAREL, Leiden (NL); Martina Gerardina Maria HUIJBERS, Leiden (NL)

(21) Appl. No.: 17/930,913

(22) Filed: Sep. 9, 2022

Related U.S. Application Data
(63) Continuation of application No. PCT/EP2021/055844, filed on Mar. 9, 2021.

(30) Foreign Application Priority Data
Mar. 10, 2020 (GB) 2003444.3

(51) Int. Cl. C07K 16/40; A61P 25/28; A61P 21/00

(52) U.S. Cl. CPC C07K 16/40; A61P 25/28; A61P 21/00; A61K 2039/505

(57) ABSTRACT

The present invention relates to a MuSK agonist antibody, or a VH, VL or CDR3 thereof, a polynucleotide encoding said antibody or a VH, VL or CDR3 thereof or an expression vector comprising said polynucleotide or a composition comprising said antibody, polynucleotide or expression vector and their use in methods of preventing and treating diseases and conditions associated with an impaired neuromuscular transmission.

### FIELD OF THE INVENTION

The present invention relates to a MuSK (Muscle-Specific kinase) agonist antibody, or a VH, VL or CDR3 thereof, a polynucleotide encoding said antibody or a VH, VL or CDR3 thereof or an expression vector comprising said polynucleotide or a composition comprising said antibody, polynucleotide or expression vector and their use in methods of preventing and treating diseases and conditions associated with an impaired neuromuscular transmission.

### BACKGROUND TO THE INVENTION

MuSK is a tyrosine kinase receptor expressed at the postsynaptic surface of the neuromuscular junction (NMJ). MuSK is an essential receptor tyrosine kinase for establishment and maintenance of the NMJ. Activation of MuSK by agrin, a neuronally derived heparan-sulfate proteoglycan, and Low Density Lipoprotein Receptor-related protein 4 (LRP4), the agrin receptor, leads to activation of a range of intracellular signaling of which one culminates in clustering of acetylcholine receptors (AChRs) on the postsynaptic side of the NMJ. AChR clustering is a prerequisite for successful neuromuscular transmission and muscle contraction. Perturbation of MuSK signaling leads to impaired neuromuscular transmission and therewith causes muscle weakness. The ectodomain of MuSK comprises of three immunoglobulin-like domains (Ig-like domain 1-3) and a cysteine-rich domain (Fz-CRD) related to those in Frizzled proteins, the receptors for Wnts. Impairment of the NMJ structure or function is believed to be one of the hallmarks of in many neuromuscular disorders amongst which myasthenia gravis (MG) and amyotrophic lateral sclerosis (ALS).

The currently approved ALS treatment (Riluzole) benefits only 20% of ALS patients by extending their life for approximately three months. The effect of Riluzole on muscle function is very limited. Moreover, ALS is considered a genetically heterogenous disease likely representing several subgroups with differing underlying pathology. There is currently no cure available, nor will patient-tailored therapies likely be able to aid all ALS patients because of the different underlying disease mechanisms.

Surprisingly, the inventors identified MuSK agonist antibodies that bypasses the need for the natural agonist agrin-LRP4 heterodimer. MuSK agonist antibodies can thereby stimulate MuSK signaling and strengthen/stabilize neuromuscular synapses. As NMJ synaptic instability is a common feature in ALS and is expected to underlie at least in part the primary loss of motor function in these patients, stabilizing and strengthening NMJs through antibody-mediated MuSK agonism is anticipated to act therapeutically. These antibodies have high affinity for (human and mouse) MuSK and have an effect on muscle function which is evidenced in the experimental part of the application by the visualization of the induction of AChR clustering. Without being bound by any theory, antibodies eliciting such feature are attractive drug candidates in a number of diseases and conditions associated with an impaired neuromuscular transmission such as ALS.

### SUMMARY OF THE INVENTION

In a first aspect, the present invention relates to a MuSK agonist antibody comprising:
a variable domain specifically binding to an epitope of MuSK;
a human antibody constant domain having an Fc domain which does not have complement effector functionality;
   and
a combination of a heavy chain variable domain (VH) and a light chain variable domain (VK) as listed in the claims.

In an embodiment of this aspect, the antibody comprises a variable heavy chain domain (VH) and a variable light chain domain (VK) wherein the VH and VK domains comprise the CDR sequences as identified in the claims.

In an embodiment of this aspect, the antibody exhibits an induction or increase of induction of acetylcholine receptor clustering at the NMJ assessed by staining for AChRs clustering at the NMJ of diaphragms of mice compared to the staining obtained without MuSK agonist antibody.

In an embodiment of this aspect, the antibody bivalently binds to MuSK.

In an embodiment of this aspect, the human antibody constant domain of this antibody is derived from a human antibody that does not have a complement effector functionality. In an embodiment of this aspect, the human antibody constant domain of this antibody is a human IgG4 constant domain.

In an embodiment of this aspect, the human antibody constant domain of this antibody is derived from a human antibody which naturally has complement effector functionality, and which contains a mutation that silences the complement effector functionality. In an embodiment of this aspect, the human antibody constant domain of this antibody is derived from human IgG1, human IgG2, or human IgG3. In a preferred embodiment, the human antibody constant domain is derived from human IgG1. In an embodiment of this aspect, the mutations of the antibody constant domains comprise L234A and L235A.

In an embodiment of this aspect, the CDRs of the antibody have been engineered to reduce immunogenicity.

In a second aspect, there is provided a polynucleotide, which is represented by a nucleotide sequence which encodes the antibody of the first aspect or a VH or VL or CDR3 domain thereof.

In an embodiment of this aspect, the nucleotide sequence encodes an antibody comprising a combination of a heavy chain variable domain (VH) and a light chain variable domain (VK) as defined in the claims.

In a third aspect, there is provided an expression vector comprising the nucleotide sequence identified in the second aspect, preferably operably linked to a regulatory sequence which allows expression of the antibody, VH or VL or CDR3 in a host cell or cell-free expression system.

In a fourth aspect, there is provided a host cell or cell-free expression system containing the expression vector of the third aspect.

In a fifth aspect, there is provided a composition comprising an antibody of the first aspect, a polynucleotide of the second aspect, an expression vector of the third aspect or a host cell or cell-free expression system of the fourth aspect.

In an embodiment of this aspect, the composition is a pharmaceutical composition comprising at least one pharmaceutically acceptable carrier or excipient.

In a further aspect, there is provided an antibody of the first aspect, a polynucleotide of the second aspect, an expression vector of the third aspect, a host cell or cell-free expression system of the fourth aspect or a pharmaceutical composition of the fifth aspect for use as a medicament. In an embodiment, the medicament is for use for treating a disease or condition associated with an impaired neuromuscular transmission. In an embodiment, such disease or condition is a neurodegenerative disease, preferably Amyotrophic Lateral Sclerosis (ALS), Alzheimer's disease, Huntington's disease, Parkinson's disease, frontotemporal dementia, Spinal Muscular Atrophy (SMA), congenital myasthenia, Emery-Dreifuss muscular dystrophy, Charcot-Marie tooth, Myasthenia Gravis, (post)poliomyelitis, aging or age-related muscle wasting, sarcopenia.

In a further aspect, there is provided a method of treating a subject in need thereof, wherein the method comprises administering to the subject a therapeutically effective amount of an antibody of the first aspect, a polynucleotide of the second aspect, an expression vector of the third aspect, a host cell or cell-free expression system of the fourth aspect or a pharmaceutical composition of the fifth aspect.

In an embodiment, such therapeutically effective amount of an antibody of the first aspect, polynucleotide of the second aspect, expression vector of the third aspect, host cell or cell-free expression system of the fourth aspect or pharmaceutical composition of the fifth aspect is administered to prevent or treat a disease or condition related with an impaired neuromuscular transmission, preferably a neurodegenerative disease, more preferably Amyotrophic Lateral Sclerosis (ALS), Alzheimer's disease, Huntington's disease, Parkinson's disease, frontotemporal dementia, Spinal Muscular Atrophy (SMA), congenital myasthenia, Emery-Dreifuss muscular dystrophy, Charcot-Marie tooth, Myasthenia Gravis, (post)poliomyelitis, aging or age-related muscle wasting, sarcopenia.

In a further aspect, there is provided a kit comprising an antibody as defined in the first aspect or a VH or VL or CDR3 domain thereof.

### BRIEF DESCRIPTION OF DRAWINGS

FIG. 1: Biological activity at increasing temperatures. The figure relates to experiment 2 method 2 of Table 5.

FIG. 2: Epifluorescence images showing ex-vivo binding of our antibodies to mouse NMJs, visualized by BTX staining of AChR.

FIG. 3: The set-up of the Biacore assay.

FIG. 4: binding of antibodies to MuSK in Mesoscale in TBS versus TBS containing 1.5 mM Ca.

FIG. 5: binding of antibodies to MuSK in Mesoscale in TBS versus TBS containing 1 mM Mg

FIG. 6: binding in Elisa to full length and truncated variants of MuSK

FIG. 7: sensogram in Biacore for binding of different antibodies to a MuSK coated chip after saturation first with 3F6c (left panel). Zooming in on binding signals (right panel).

FIG. 8: domain structure of MuSK and epitope of the different antibodies

FIG. 9: graph presenting AChR clusters larger or equal to 15 pmt (left panel) or larger or equal to 3 pmt (right panel) compared to agrin upon incubation with a dose-concentration range of pre-lead agonistic MuSK Ab. (All numbers are averages normalized against an agrin control and N=3 unless otherwise specified in Tables 25-26)

FIG. 10: graph presenting AChR clusters larger or equal to 15 pmt compared to agrin upon incubation of MG-derived polyclonal Antibodies and pre-lead agonistic MuSK Ab. (All numbers are averages normalized against an agrin control and N=4 unless otherwise specified)

FIG. 11: graph presenting AChR clusters larger or equal to 3 pmt compared to agrin upon incubation of MG-derived polyclonal Antibodies and pre-lead agonistic MuSK Ab (numbers are averages normalized against an agrin control and N=4 unless otherwise specified)

FIG. 12: AChR staining of diaphragm NMJs with AF488-BTx of two untreated mice (5 representative images per mouse). Upper panel male, lower panel female

FIG. 13: AChR staining of diaphragm NMJs with AF488-BTx of two 3B5 treated mice (5 representative images per mouse). Upper panel male, lower panel female

FIG. 14: AChR staining of diaphragm NMJs with AF488-BTx of two 3D9b treated mice (5 representative images per mouse). Upper panel male, lower panel female. Some synapses appear normal while other show a striping pattern

FIG. 15: AChR staining of diaphragm NMJs with AF488-BTx of two 3F6c treated mice (5 representative images per mouse). Upper panel male, lower panel female.

FIG. 16: AChR staining of diaphragm NMJs with AF488-BTx of two 4D3 treated mice (5 representative images per mouse). Upper panel male, lower panel female.

FIG. 17: AChR staining of diaphragm NMJs with AF488-BTx of two 3D10a treated mice (5 representative images per mouse). Upper panel male, lower panel female.

### DETAILED DESCRIPTION

### A. Definitions

Unless defined otherwise, all technical and scientific terms used herein have the same meaning as is commonly understood by one skilled in the art in the technical field of the invention.

“Agonist”—As used herein, the term “agonist” means any agent capable of binding to MuSK. By binding to MuSK, the agonists of the invention are able to activate MuSK or able to elicit an agonistic MuSK activity as explained later herein. Agonists in accordance with the present invention will typically bind specifically or “specifically bind” to MuSK. The term “specifically bind” refers to the ability of an agonist to preferentially bind to its target MuSK. In an embodiment, an agonist binds or specifically binds MuSK without agrin. In the context of the invention “specifically binding” means the binding of an agent, preferably an antibody to a predetermined epitope of a target (i.e. MuSK) with an affinity corresponding to a KD (equilibrium dissociation constant) of 10-2 M or less, or 10-3 M or less, or 10-4 M or less, or 10-5 M or less, or 10-6 M or less, 10-7 M or less, or 10-8 M or less, 10-9 M or less, or 10-10 M or less, when determined by surface plasma resonance (SPR) technology in a Biacore 3000 instrument using the antibody as the analyte (wherein a low KD indicates a high affinity). Alternatively the assay used is an ELISA as used in the experimental part. Agents capable of binding to protein targets, particularly agents capable of exhibiting binding specificity for a given protein target, are known to those skilled in the art. Such agents include but are not limited to small molecule, biological molecules including peptides, and antibody mimetics such as affibodies, affilins, affitins, adnectins, atrimers, evasins, DARPins, anticalins, avimers, fynomers, versabodies and duocalins. Preferred agonists in accordance with the present invention are antibodies. The wording “agonist” may be replaced by “agonist agent” and when the agent is an antibody by “agonist antibody” Therefore in the context of the invention, a “MuSK agonist antibody” may be replaced by an “agonist”.

“Antibody” or “Immunoglobulin”—As used herein, the term “immunoglobulin” includes a polypeptide having a combination of two heavy and two light chains whether or not it possesses any relevant specific immunoreactivity. “Antibodies” refer to such assemblies which have significant known specific immunoreactive activity to an antigen of interest (herein MuSK). The term “MuSK antibodies” is used herein to refer to antibodies which exhibit immunological specificity for the MuSK protein, including human MuSK, and in some cases species homologues thereof. Antibodies and immunoglobulins comprise light and heavy chains, with or without an interchain covalent linkage between them. Basic immunoglobulin structures in vertebrate systems are relatively well understood.

The generic term “immunoglobulin” comprises five distinct classes of antibody that can be distinguished biochemically. All five classes of antibodies are within the scope of the present invention. The following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

The light chains of an antibody are classified as either kappa or lambda (KA. Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention.

The variable region (or variable domain) of an antibody allows the antibody to selectively recognize and specifically bind an epitope on an antigen. That is, the VL domain and VH domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the VH and VL chains. As defined herein, the variable region specifically binds an epitope of MuSK, said variable region comprises three CDRs on each of the VH and VL chains or VH and VKappa (VK) chains.

“MuSK” As used herein, the term “MuSK” stands for “Muscle-specific kinase”. The term “MuSK” is synonymous of “MuSK protein”. MuSK is a receptor tyrosine kinase expressed in muscle cells and enriched at the neuromuscular junction (NMJ) or at AChR dense plaques on myotubes in culture. The MuSK protein is characterized by the presence of three Ig-like domains (Ig-like 1, Ig-like 2 and Ig-like 3), a domain called Fz-like domain, a transmembrane domain and an intracellular domain comprising a tyrosine kinase. The stimulation of MuSK triggers a signal at the NMJ: neuromuscular transmission. This signal is essential for the maintenance of the structure and the function of the neuromuscular junction. Without being bound to any theory, it is believed that the stimulation/activation of MuSK is mimicked by the agonist of the invention as a result of its binding to an epitope of MuSK. The agonists of the invention are expected to be able to elicit an agonistic MuSK activity. In the context of the application, an agonistic MuSK activity is synonymous with a MuSK activity. In the context of the application, an agonistic MuSK activity may be replaced by the triggering of a MuSK-induced signal in a muscle cell at the NMJ. In an embodiment, the agonist can trigger MuSK in the absence of agrin or independently of agrin. A MuSK-induced signal may be at least one of the induction of Musk dimerization, the induction of MuSK tyrosine phosphorylation, the induction of AChRs clustering, the increase of the number of fully innervated NMJ, the decrease of the number of fully denervated NMJ, an improvement of the reliability of synaptic transmission, a reduction/decrease of motor neuron death, an improved motor function, a stabilization of NMJ, an extension of the lifespan of a treated subject.

In a preferred embodiment, an agonistic MuSK activity is to exhibit an induction or increase of induction of AChRs clustering at the NMJ assessed by staining for AChRs clustering at the NMJ of diaphragms of mice compared to the staining obtained without MuSK agonist antibody or obtained with a control antibody. In an embodiment, this induction or increase of clustering of AChRs at the NMJ results in a more normal/physiological NMJ morphology maintaining synaptic innervation and/or pre- and post-synaptic alignment.

The term “MuSK” encompasses the human protein and any homologues species thereof. The amino acid sequence of the extracellular domain of the human MuSK is represented by SEQ ID NO: 1 (Accession number Uniprot 015146-1, R&D Systems). Within this amino acid sequence, the human MuSK Ig-like 1 domain is from amino acid 28-116 (SEQ ID NO: 15), the human MuSK Ig-like 2 domain is from amino acid 121-205 (SEQ ID NO: 16), the human MuSK Ig-like 3 domain is from amino acid 212-302 (SEQ ID NO: 17), and the human MuSK Fz domain is from amino acid 312-450 (SEQ ID NO: 18). Also encompassed within the term “MuSK” are naturally occurring variants of the human sequence.

“Epitope”—As used herein, the term “epitope” means the region of the MuSK protein to which the agonist binds. An agonist will typically bind to its respective MuSK epitope via a complementary binding site on the agonist. The epitope to which the agonist binds will typically comprise one or more amino acids from the full-length MuSK protein. In an embodiment, an epitope comprises 5 to 20, 5 to 15, 5 to 10 contiguous amino acids of MuSK protein. The epitope may include amino acids that are contiguous in the MuSK protein i.e. a linear epitope or may include amino acids that are non-contiguous in the MuSK protein i.e. a conformational epitope.

“Binding Site”—As used herein, the term “binding site” comprises a region of a polypeptide which is responsible for selectively binding to a target antigen of interest (e.g. MuSK). As used herein, the term “monovalent” when referring to a polypeptide as disclosed herein denotes a polypeptide in monomeric form e.g. contains only one binding site for MuSK. A monovalent polypeptide as disclosed herein may comprise at least one CDR as disclosed herein and preferably comprises or consists of a combination of CDRs as disclosed herein. In an embodiment, a monovalent polypeptide comprises or consists of one immunoglobulin single variable domain as disclosed herein. In an embodiment, a monovalent polypeptide is an antibody as disclosed herein.

As used herein, the term “bivalent” when referring to a polypeptide as disclosed herein denotes a polypeptide that comprises or consists of two o immunoglobulin single variable domains, which may be the same or different, and may be directed against the same antigen or antigenic determinant or may be alternatively be directed against different antigenic determinants; or any suitable combinations thereof. A bivalent polypeptide comprises or consists of two binding sites. In an embodiment, a bivalent polypeptide comprises or consists of one or two immunoglobulin single variable domain as disclosed herein. In an embodiment, a bivalent polypeptide is an antibody as disclosed herein.

As used herein, the term “multivalent” when referring to a polypeptide as disclosed herein denotes a polypeptide that comprises or consists of two or more immunoglobulin single variable domains, which may be the same or different, and may be directed against the same antigen or antigenic determinant or may be alternatively be directed against different antigenic determinants; or any suitable combinations thereof. A multivalent polypeptide comprises or consists of two or more binding sites. Binding domains comprise at least one binding site. Exemplary binding domains include an antibody variable domain. The antibody molecules of the invention may comprise a single binding site or multiple (e.g., two, three or four) binding sites. In an embodiment, the MuSK agonist antibody of the invention bivalently or multivalently binds to MuSK.

“Derived From”—As used herein the term “derived from” a designated protein (e.g. a camelid antibody or antigen binding fragment thereof) refers to the origin of the polypeptide or amino acid sequence. In one embodiment, the polypeptide or amino acid sequence which is derived from a particular starting polypeptide is a CDR sequence or sequence related thereto. In one embodiment, the amino acid sequence which is derived from a particular starting polypeptide is not contiguous. For example, in one embodiment, one, two, three, four, five, or six CDRs are derived from a starting antibody.

In one embodiment, the polypeptide or amino acid sequence which is derived from a particular starting polypeptide or amino acid sequence has an amino acid sequence that is essentially identical or is identical to that of the starting sequence, or a portion thereof wherein the portion consists of at least 3-5 amino acids, at least 3-7 amino acids, at least 5-10 amino acids, at least 5-15 amino acids, at least 10-20 amino acids, at least 20-30 amino acids, or at least 30-50 amino acids, or which is otherwise identifiable to one of ordinary skill in the art as having its origin in the starting sequence.

In one embodiment, the polypeptide or amino acid sequence which is derived from a particular starting polypeptide or amino acid sequence has an amino acid sequence that comprises at least 80%, 81%, 82%, 83%, 84%, 85%, 86%, 87%, 88%, 89%, 90%, 91%, 92%, 93%, 94%, 95%, 96%, 97%, 98%, 99% identity with a given CDR, VH, VL, VK sequence and still retains a biological activity of the CDR, VH, VL, VK it derived from at least to some extent. Identity is preferably over the full length of a given CDR, VH, VL, VK or over a portion thereof as indicated herein.

In one embodiment, the one or more CDR sequences derived from the starting antibody are altered to produce variant CDR sequences, e.g. affinity variants, wherein the variant CDR sequences maintain target antigen binding activity at least to some extent. In this context, “at least some extent” means at least 50%, at least 60%, at least 70%, at least 80%, at least 90%, or at least 91% or at least 92% or at least 93% or at least 94% or at least 95% or at least 96% or at least 97% or at least 98% or at least 99% or 100%. The assay and the activity which may be compared are defined herein.

“Camelid-Derived”—In certain preferred embodiments, the antibodies of the invention comprise framework amino acid sequences and/or CDR amino acid sequences derived from a camelid conventional antibody raised by active immunisation of a camelid. However, antibodies of the invention comprising camelid-derived amino acid sequences may be engineered to comprise framework and/or constant region sequences derived from a human amino acid sequence (i.e. a human antibody) or other non-camelid mammalian species. For example, a human or non-human primate framework region, heavy chain portion, and/or hinge portion may be included in the MuSK antibodies. In one embodiment, one or more non-camelid amino acids may be present in the framework region of a “camelid-derived” antibody, e.g., a camelid framework amino acid sequence may comprise one or more amino acid mutations in which the corresponding human or non-human primate amino acid residue is present. Moreover, camelid-derived VH and VL (or VH and VK) domains, or humanised variants thereof, may be linked to the constant domains of human antibodies to produce a chimeric molecule, as described elsewhere herein.

“Conservative amino acid substitution”—A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide may be replaced with another amino acid residue from the same side chain family. In another embodiment, a string of amino acids can be replaced with a structurally similar string that differs in order and/or composition of side chain family members.

“Heavy chain portion”—As used herein, the term “heavy chain portion” includes amino acid sequences derived from the constant domains of an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. In one embodiment, an antibody or antigen binding fragment of the invention may comprise the Fc portion of an immunoglobulin heavy chain (e.g., a hinge portion, a CH2 domain, and a CH3 domain). In another embodiment, an antibody or antigen binding fragment of the invention may lack at least a portion of a constant domain (e.g., all or part of a CH2 domain). In certain embodiments, at least one, and preferably all, of the constant domains are derived from a human immunoglobulin heavy chain. For example, in one preferred embodiment, the heavy chain portion comprises a fully human hinge domain. In other preferred embodiments, the heavy chain portion comprising a fully human Fc portion (e.g., hinge, CH2 and CH3 domain sequences from a human immunoglobulin).

In certain embodiments, the constituent constant domains of the heavy chain portion are from different immunoglobulin molecules. For example, a heavy chain portion of a polypeptide may comprise a CH2 domain derived from an IgG1 molecule and a hinge region derived from an IgG3 or IgG4 molecule. In other embodiments, the constant domains are chimeric domains comprising portions of different immunoglobulin molecules. For example, a hinge may comprise a first portion from an IgG1 molecule and a second portion from an IgG3 or IgG4 molecule. As set forth above, it will be understood by one of ordinary skill in the art that the constant domains of the heavy chain portion may be modified such that they vary in amino acid sequence from the naturally occurring (wild-type) immunoglobulin molecule. That is, the polypeptides of the invention disclosed herein may comprise alterations or modifications to one or more of the heavy chain constant domains (CH1, hinge, CH2 or CH3) and/or to the light chain constant region domain (CL). Exemplary modifications include additions, deletions or substitutions of one or more amino acids in one or more domains.

“Chimeric”—A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship. Exemplary chimeric antibodies of the invention include fusion proteins comprising camelid-derived VH and VL domains, or humanised variants thereof, fused to the constant domains of a human antibody, e.g. human IgG1, IgG2, IgG3 or IgG4.

“Variable region” or “variable domain”—The terms “variable region” and “variable domain” are used herein interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20:267-279 (2000)). The first, second and third hypervariable loops of the VKappa light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20:267-279 (2000)).

Unless otherwise indicated, the terms L1, L2 and L3 respectively refer to the first, second and third hypervariable loops of a VL domain, and encompass hypervariable loops obtained from both Vkappa (VK) and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, Md., 1983) and the limits of the HVs and the CDRs may be different in some VH and VL domains.

The CDRs of the VL and VH domains can typically be defined as comprising the following amino acids: residues 24-34 (LCDR1), 50-56 (LCDR2) and 89-97 (LCDR3) in the light chain variable domain, and residues 31-35 or 31-35b (HCDR1), 50-65 (HCDR2) and 95-102 (HCDR3) in the heavy chain variable domain; (Kabat et al., Sequences of Proteins of Immunological Interest, 5th Ed. Public Health Service, National Institutes of Health, Bethesda, Md. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215:175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

“CDR”—As used herein, the term “CDR” or “complementarity determining region” means the non-contiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 1
CDR definitions

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

“Framework region”—The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable domain and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or

McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Hinge region”—As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux K. H. et al. J. Immunol. 161:4083-90 1998). Antibodies of the invention comprising a “fully human” hinge region may contain one of the hinge region sequences shown in Table 2 below.

TABLE 2
Human hinge sequences

| IgG | Upper hinge | Middle hinge | Lower hinge |
|---|---|---|---|
| IgG1 | EPKSCDKTHT | CPPCP | APELLGGP |
| | (SEQ ID NO: 2) | (SEQ ID NO: 3) | (SEQ ID NO: 4) |
| IgG3 | ELKTPLGDTTHT | CPRCP (EPKSCDT | APELLGGP |
| | (SEQ ID NO: 5) | PPPCPRCP)₃ | (SEQ ID NO: 7) |
| | | (SEQ ID NO: 6) | |
| IgG4 | ESKYGPP | CPSCP | APEFLGGP |
| | (SEQ ID NO: 8) | (SEQ ID NO: 9) | (SEQ ID NO: 10) |
| IgG2 | ERK | CCVECPPCP | APPVAGP |
| | (SEQ ID NO: 11) | (SEQ ID NO: 12) | (SEQ ID NO: 13) |

“CH2 domain”—As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system, Kabat E A et al. Sequences of Proteins of Immunological Interest. Bethesda, US Department of Health and Human Services, NIH. 1991). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

“Fragment”—The term “fragment”, as used in the context of antibodies of the invention, refers to a part or portion of an antibody or antibody chain comprising fewer amino acid residues than an intact or complete antibody or antibody chain. The term “antigen binding fragment” refers to a polypeptide fragment of an immunoglobulin or antibody that binds antigen or competes with intact antibody (i.e., with the intact antibody from which they were derived) for antigen binding (i.e., specific binding to MuSK). As used herein, the term “fragment” of an antibody molecule includes antigen binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, a one-armed (monovalent) antibody, diabodies, triabodies, tetrabodies or any antigen binding molecule formed by combination, assembly or conjugation of such antigen binding fragments. The term “antigen binding fragment” as used herein is further intended to encompass antibody fragments selected from the group consisting of unibodies, domain antibodies and nanobodies. Fragments can be obtained, e.g., via chemical or enzymatic treatment of an intact or complete antibody or antibody chain or by recombinant means.

“scFv” or “scFv fragment”—An scFv or scFv fragment means a single chain variable fragment. An scFv is a fusion protein of a VH domain and a VL domain of an antibody connected via a linker.

“Valency”—As used herein the term “valency” refers to the number of potential target binding sites in a polypeptide (i.e. antibody). Each target binding site specifically binds one target molecule or specific site on a target molecule (i.e. MuSK). When a polypeptide comprises more than one target binding site, each target binding site may specifically bind the same or different molecules (e.g., may bind to different ligands or different antigens, or different epitopes on the same antigen).

“Specificity”—The term “specificity” refers to the ability to bind (e.g., immunoreact with) a given target, e.g. MuSK. A polypeptide (i.e. antibody) may be monospecific and contain one or more binding sites which specifically bind a target or a polypeptide may be multispecific and contain two or more binding sites which specifically bind the same or different targets.

“Synthetic”—As used herein the term “synthetic” with respect to polypeptides includes polypeptides which comprise an amino acid sequence that is not naturally occurring. For example, non-naturally occurring polypeptides which are modified forms of naturally occurring polypeptides (e.g., comprising a mutation such as an addition, substitution or deletion) or which comprise a first amino acid sequence (which may or may not be naturally occurring) that is linked in a linear sequence of amino acids to a second amino acid sequence (which may or may not be naturally occurring) to which it is not naturally linked in nature. In an embodiment, the MuSK agonistic antibody of the invention are synthetic, as they do not occur in nature.

“Engineered”—As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the antibodies of the invention are engineered, including for example, humanized and/or chimeric antibodies, and antibodies which have been engineered to improve one or more properties, such as antigen binding, stability/half-life or effector function. In an embodiment, the term “engineered” is applied to a cell as identified herein.

“Modified antibody”—As used herein, the term “modified antibody” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. scFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen). In another embodiment, a modified antibody of the invention is a fusion protein comprising at least one heavy chain portion lacking a CH2 domain and comprising a binding domain of a polypeptide comprising the binding portion of one member of a receptor ligand pair.

The term “modified antibody” may also be used herein to refer to amino acid sequence variants of the antibodies of the invention as structurally defined herein. It will be understood by one of ordinary skill in the art that an antibody may be modified to produce a variant antibody which varies in amino acid sequence in comparison to the antibody from which it was derived. For example, nucleotide or amino acid substitutions leading to conservative substitutions or changes at “non-essential” amino acid residues may be made (e.g., in CDR and/or framework residues). Amino acid substitutions can include replacement of one or more amino acids with a naturally occurring or non-natural amino acid.

“Humanising substitutions”—As used herein, the term “humanising substitutions” refers to amino acid substitutions in which the amino acid residue present at a particular position in the VH or VL domain of an antibody (for example a camelid-derived MuSK antibody) is replaced with an amino acid residue which occurs at an equivalent position in a reference human VH or VL domain. The reference human VH or VL domain may be a VH or VL domain encoded by the human germline. Humanising substitutions may be made in the framework regions and/or the CDRs of the antibodies, defined herein. Preferably a CDR or all CDRs have been engineered to reduce immunogenicity. A reduced immunogenicity can be obtained by the introduction of humanising substitutions.

“Humanised variants”—As used herein the term “humanised variant” refers to a variant antibody which contains one or more “humanising substitutions” compared to a reference antibody, wherein a portion of the reference antibody (e.g. the VH domain and/or the VL domain or parts thereof containing at least one CDR) has an amino acid derived from a non-human species, and the “humanising substitutions” occur within the amino acid sequence derived from a non-human species.

“Germlined variants”—The term “germlined variant” is used herein to refer specifically to “humanised variants” in which the “humanising substitutions” result in replacement of one or more amino acid residues present at a particular position (s) in the VH or VL or VK domain of an antibody (for example a camelid-derived MuSK antibody) with an amino acid residue which occurs at an equivalent position in a reference human VH or VL or VK domain encoded by the human germline. It is typical that for any given “germlined variant”, the replacement amino acid residues substituted into the germlined variant are taken exclusively, or predominantly, from a single human germline-encoded VH or VL or VK domain. The terms “humanised variant” and “germlined variant” are often used interchangeably herein. Introduction of one or more “humanising substitutions” into a camelid-derived (e.g. llama derived) VH or VL or VK domain results in production of a “humanised variant” of the camelid (llama)-derived VH or VL or VK domain. If the amino acid residues substituted in are derived predominantly or exclusively from a single human germline-encoded VH or VL or VK domain sequence, then the result may be a “human germlined variant” of the camelid (llama)-derived VH or VL or VK domain.

“Affinity variants”—As used herein, the term “affinity variant” refers to a variant antibody which exhibits one or more changes in amino acid sequence compared to a reference antibody, wherein the affinity variant exhibits an altered affinity for the target antigen in comparison to the reference antibody. For example, affinity variants will exhibit a changed affinity for MuSK, as compared to the reference MuSK antibody. Preferably the affinity variant will exhibit improved affinity for the target antigen, e.g. MuSK, as compared to the reference antibody. Affinity variants typically exhibit one or more changes in amino acid sequence in the CDRs, as compared to the reference antibody. Such substitutions may result in replacement of the original amino acid present at a given position in the CDRs with a different amino acid residue, which may be a naturally occurring amino acid residue or a non-naturally occurring amino acid residue. The amino acid substitutions may be conservative or non-conservative.

“High human homology”—In the context of the invention, “high human homology” means “substantially human”. An antibody comprising a heavy chain variable domain (VH) and a light chain variable domain (VL) or (VK) may be considered as having high human homology if the (VH) domains and the (VL) or (VK) domains, taken together, exhibit at least 90%, at least 91%, at least 92%, at least 93%, at least 94%, at least 95%, at least 96%, at least 97%, at least 98%, at least 99% or 100% amino acid sequence identity to the closest matching human germline VH and VL (or VH and VK) sequences. Antibodies having high human homology may include antibodies comprising VH and VL (or VH and VK) domains of native non-human antibodies which exhibit sufficiently high percent sequence identity to human germline sequences (such as at least 90%, at least 91%, at least 92%, at least 93%, at least 94%, at least 95%, at least 96%, at least 97%, at least 98%, at least 99% or 100% amino acid sequence identity), including for example antibodies comprising VH and VL (or VH and VK) domains of camelid conventional antibodies, as well as engineered, especially humanised or germlined, variants of such antibodies and also “fully human” antibodies.

In one embodiment the VH domain of the antibody with high human homology may exhibit an amino acid sequence identity or sequence homology at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with one or more human VH domains across the framework regions FR1, FR2, FR3 and FR4 and/or across the CDR1, CDR2 and/or CDR3 of said VH domain. In other embodiments the amino acid sequence identity or sequence homology between the VH domain of the polypeptide of the invention and the closest matching human germline VH domain sequence may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%.

In one embodiment the VH domain of the antibody with high human homology may contain one or more (e.g. 1 to 10, or 1 or 2 or 3 or 4 or 5 or 6 or 7 or 8 or 9 or 10) amino acid sequence mis-matches across the framework regions FR1, FR2, FR3 and FR4, in comparison to the closest matched human VH sequence.

In another embodiment the VL (or VK) domain of the antibody with high human homology may exhibit a sequence identity or sequence homology of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with one or more human VL (or VK) domains across the framework regions FR1, FR2, FR3 and FR4 and/or across the CDR1, CDR2 and/or CDR3 of said VL domain. In other embodiments the amino acid sequence identity or sequence homology between the VL (or VK) domain of the polypeptide of the invention and the closest matching human germline VL (or VK) domain sequence may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%.

In one embodiment the VL (or VK) domain of the antibody with high human homology may contain one or more (e.g. 1 to 10 or 1 or 2 or 3 or 4 or 5 or 6 or 7 or 8 or 9 or 10) amino acid sequence mis-matches across the framework regions FR1, FR2, FR3 and FR4, in comparison to the closest matched human VL (or VK) sequence.

### B. MuSK Agonists

In a first aspect, the present invention provides a MuSK agonist, which binds to a MuSK, wherein the agonist binds to an epitope of a MuSK. The agonists of the invention preferably bind to human MuSK as earlier defined herein (SEQ ID NO:1). SEQ ID NO:1 is the extracellular domain of human MuSK (Uniprot, 015146). In an embodiment, the agonist of the invention is an antibody. The wording “agonist” in the context of the antibody of the invention may also be replaced by the wording “activating”. In an embodiment, the binding or the specific binding is in solution and in a cell-free environment. In another embodiment, the binding or specific binding is a binding or a specific binding to a MuSK expressed in a cell (in vivo, in vitro or ex vivo). The cell is preferably a muscle cell. Preferably, the MuSK is expressed at the cellular membrane of a cell, preferably at the NMJ. A cell may be a C2C12 cell line (ATCC® CRL-1772™, Sigma Catalogus number 91031101) or a HEK cell line expressing MuSK (HEK293E-253). MuSK may also be produced in HEK293 (ATCC® CRL-1573™) or bought from R&D Systems. In an embodiment, the specific binding is assessed as earlier defined herein.

In an embodiment, the specific binding is assessed in the absence of agrin.

In an embodiment, the specific binding is assessed in a competitive assay. In an embodiment, the binding of such agonist antibody is assessed in parallel with the binding of an anti-MuSK autoantibodies from a MG patient. In an embodiment, such antibody has been described in Huijbers M G et al PNAS 2013, 110(51): 20783-20788. In a preferred embodiment, the affinity of the binding of the agonist antibody to MuSK is stronger than the one of the autoantibody from a MG patient. In this context, stronger may mean at least 10 times, 50 times or 100 times stronger.

In a first aspect, there is provided a MuSK agonist antibody comprising:
A variable domain specifically binding to an epitope of MuSK;
A human antibody constant domain having an Fc domain which does not have complement effector functionality; and:
a combination of a heavy chain variable domain (VH) and a light chain variable domain (VK) selected from the following:
   (d1) a VH comprising the amino acid sequence of SEQ ID NO:37 or an amino acid sequence at least 90%, 95%, 97%, 98% or 99% identical thereto and a VK comprising the amino acid sequence of SEQ ID NO:57 or an amino acid sequence at least 90%, 95%, 97%, 98% or 99% identical thereto (3F6c);
   (d2) a VH comprising the amino acid sequence of SEQ ID NO:36 or an amino acid sequence at least 90%, 95%, 97%, 98% or 99% identical thereto and a VK comprising the amino acid sequence of SEQ ID NO:56 or an amino acid sequence at least 90%, 95%, 97%, 98% or 99% identical thereto (3D10a); or
   (d3) a VH comprising the amino acid sequence of SEQ ID NO:38 or an amino acid sequence at least 90%, 95%, 97%, 98% or 99% identical thereto and a VK comprising the amino acid sequence of SEQ ID NO:58 or an amino acid sequence at least 90%, 95%, 97%, 98% or 99% identical thereto (4D3).

The wording “a variable domain specifically binds to an epitope of MuSK” above may be replaced by the wording a MuSK agonist antibody specifically binds to an epitope of MuSK.

The epitope of the MuSK agonist antibody may be a linear epitope i.e. it may consist of two or more consecutive amino acids in the MuSK primary protein sequence (preferably SEQ ID NO:1). Alternatively, the epitope may be a conformational epitope comprising or consisting of two or more amino acids that are not located adjacent to each other in the MuSK primary protein sequence (preferably SEQ ID NO:1). For embodiments in which the agonist binds to a conformational epitope, the two or more amino acids of the epitope will typically be located in close proximity within the 3-dimensional structure of the MuSK protein. The epitopes to which the MuSK agonists of the invention bind may comprise or consist of at least two amino acids, at least three amino acids, at least four amino acids, at least five amino acids, at least six amino acids, at least seven amino acids. In certain embodiments, the epitopes to which the MuSK agonists bind comprise or consist of 5, 6, 7, 8, 9, 10, 11, 12, 13, 14, 15, 16, 17, 18, 19 or 20 amino acids. In an embodiment, the epitopes to which the MuSK agonists bind comprise or consist of 5, 6, 7, 8, 9, 10, 11, 12, 13, 14, 15, 16, 17, 18, 19 or 20 amino acids of SEQ ID NO:1.

A preferred region of SEQ ID NO:1 that is specifically bound by the MuSK agonist comprises amino acid 28-116 of SEQ ID NO:1 or is comprised within amino acid 28-116 of SEQ ID NO:1 (i.e. SEQ ID NO: 15). This region of SEQ ID NO:1 is the Ig-like 1 domain of MuSK.

Another region of SEQ ID NO:1 that may be specifically bound by the MuSK agonist comprises amino acid 121-205 of SEQ ID NO:1 or is comprised within amino acid 121-205 of SEQ ID NO:1 (i.e. SEQ ID NO: 16). This region of SEQ ID NO:1 is the Ig-like 2 domain of MuSK.

Another region of SEQ ID NO:1 that may be specifically bound by the MuSK agonist comprises amino acid 212-302 of SEQ ID NO:1 or is comprised within amino acid 212-302 of SEQ ID NO:1 (i.e. SEQ ID NO: 17). This region of SEQ ID NO:1 is the Ig-like 3 domain of MuSK.

Another region of SEQ ID NO:1 that may be specifically bound by the MuSK agonist comprises amino acid 312-450 of SEQ ID NO:1 or is comprised within amino acid 312-450 of SEQ ID NO:1 (i.e. SEQ ID NO: 18). This region of SEQ ID NO:1 is the Frizzle domain of MuSK

Therefore, in certain embodiments, the agonist antibody binds to an epitope which is comprised in an Ig-like domain of MuSK. In an embodiment, the Ig-like domain is Ig-like 1, Ig-like 2 and/or Ig-like 3 as identified above. In an embodiment, the epitope bound by the MuSK agonist antibody is comprised in the Ig-like 1 domain of MuSK.

In an embodiment, the agonist of the invention binds to an epitope of MuSK. By binding to an epitope of MuSK, the agonists of the invention are able to elicit an agonistic MuSK activity. Within the context of the application “elicit an agonistic MuSK activity” may be replaced by “activate MuSK”. An agonistic MuSK activity or an activation of MuSK may be triggered at the molecular and/or at the cellular level and/or in a more biological complex system as a NMJ, a synapse, a living organism. In the context of the application, an agonistic MuSK activity may be replaced by the triggering of a MuSK-induced signal or by the induction of MuSK activation in a muscle cell at the NMJ. A MuSK-induced signal (or MuSK activation or MuSK activity) may be at least one of the induction of MuSK dimerization, the induction of MuSK tyrosine phosphorylation, the induction or increase of induction of AChRs clustering at the NMJ (or clustering in vitro in myotubes AChR patches), the increase of the number of fully innervated NMJ, the decrease of the number of fully denervated NMJ, an improvement of the reliability of synaptic transmission, a prevention/stabilization or even a reduction/decrease of motor neuron death, an extension of the lifespan of a treated subject.

A MuSK-induced signal by the agonist antibody of the invention may be the induction of MuSK dimerization may be assessed by western blotting using an antibody against MuSK. In the context of the invention, an agonistic activity of MuSK may have been assessed when the induction of MuSK dimerization is increased of at least 10%, 20%, 30%, 40%, 50%, 60%, 70%, 80%, 90%, 100% or more in an experiment using the antibody of the invention by comparison with the same experimental setting without any antibody or with a negative control or with a negative control antibody as defined later herein. Alternatively, in the context of the invention, an agonistic activity of MuSK antibody may have been assessed when the induction of MuSK dimerization is the same or about the same (20% less, 10% less or the same or 10% more or 20% more) in an experiment using the antibody of the invention by comparison with the same experimental setting without a positive control antibody as defined later herein. Such a MuSK dimerization may be assessed without agrin. A positive control in the assessment of MuSK dimerization is agrin.

A MuSK-induced signal by the agonist antibody of the invention may be the induction of MuSK tyrosine phosphorylation and such phosphorylation may be assessed by western blotting using an antibody specific for tyrosine phosphorylation. In the context of the invention, an agonistic activity of MuSK may have been assessed when the induction of MuSK tyrosine phosphorylation is increased of at least 10%, 20%, 30%, 40%, 50%, 60%, 70%, 80%, 90%, 100%, 120%, 150%, 180%, 200% or more in an experiment using the antibody of the invention by comparison with the same experimental setting without any antibody or with a negative control as defined later herein. Alternatively, in the context of the invention, an agonistic activity of MuSK may have been assessed when the induction of MuSK tyrosine phosphorylation is the same or about the same (20% less, 10% less or the same or 10% more or 20% more) in an experiment using the antibody of the invention by comparison with the same experimental setting without a positive control antibody as defined later herein. Such a MuSK tyrosine phosphorylation may be assessed without agrin. A positive control in the assessment of MuSK tyrosine phosphorylation is agrin.

A MuSK-induced signal by the agonist antibody of the invention may be the induction of acetylcholine receptor (AChR) clustering at the NMJ and such clustering may be assessed by staining of AChR using an antibody specifically binding to AChR and visualising such staining in fluorescent microscopy using techniques known to the skilled person. Alternatively, the clustering may be assessed in vitro in myotubes AChR patches. A preferred antibody used to visualise AChR clustering is an antibody specific for AChR. More preferred antibody is AlexaFluor488 conjugated α-bungarotoxin (B13422, ThermoFisher). Usually the region to be analysed in fixed in paraformaldehyde and incubated at room temperature with the relevant antibody of the invention or with a positive or negative control and subsequently each region is washed with PBS and observed under an epi-fluorescent microscopy. In the context of the invention, an agonistic activity of MuSK may have been assessed when the induction of AChR clustering at the NMJ is the same or about the same (i.e. 20% less, 10% less or the same or 10% more or 20% more) or is increased of at least 10%, 20%, 30%, 40%, 50%, 60%, 70%, 80%, 90%, 100% in an experiment using the antibody of the invention by comparison with the same experimental setting without any antibody or with a positive control or with a negative control as defined later herein. Such a AChR clustering may be assessed without agrin. A positive control in the assessment of AChR clustering is agrin. A negative control may be an autoantibody from a MG patient.

In a preferred embodiment, the agonist antibody of the invention exhibits an induction or increase of induction of acetylcholine receptor clustering at the NMJ and such clustering may be assessed by visualizing a staining or an increased staining for AChRs at the NMJ of diaphragms of mice compared to the staining obtained without MuSK agonist antibody. This effect has been demonstrated at the NMJ of the diaphragm of NOD mice in the experimental part. In an embodiment, this induction or increase of clustering of AChRs at the NMJ results in a more normal/physiological NMJ morphology maintaining synaptic innervation and/or pre- and post-synaptic alignment.

In an embodiment, the agonist antibody does not cause AChR fragmentation upon repeated injection in vivo. In this context, “repeated” may mean 2 times, 3 times, 4 times, 5 times or more. In an embodiment, this induction or increase of clustering of AChRs at the NMJ results in a more normal/physiological NMJ morphology maintaining synaptic innervation and/or pre- and post-synaptic alignment and does not cause AChR fragmentation upon repeated injection.

A MuSK-induced signal by the agonist antibody of the invention in a muscle cell at the NMJ may be the increase of the number of fully innervated NMJ, the decrease of the number of fully denervated NMJ, an improvement of the reliability of synaptic transmission, a prevention/stabilization or even a reduction/decrease of motor neuron death. Each of these features could be assessed using techniques known to the skilled person such as staining of AChR using the a-bungarotoxin antibody as earlier defined herein, presynaptic labelling and quantifying innervation by fluorescent confocal microscopy, EMG single fibre EMG, electrophysiology of single synapses, staining of motor neuron cell bodies in bone marrow specific regions. All these assays have been described in Cantor S et al 2018 (Elife, 2018; 7:e34375).

A MuSK-induced signal or effect by the agonist antibody of the invention may be characterized by the extension of the lifespan of a treated subject. The extension may be of at least 1 day, 1 week, 2 weeks, 3 weeks, 4 weeks, 1 month, 2 months, 3 months, 4 months, 5 months, 6 months, 7 months, 8 months, 9 months, 10 months, 11 months, 1 year or more. This is assessed in comparison with the expected lifespan of a subject suffering from the same condition and having not been treated with an antibody of the invention. In this context, the subject may be an animal.

The agonistic properties of the MuSK agonists described herein may be measured in accordance with the assays described herein. An activating activity of a MuSK agonist antibody may be measured relative to a control, for example a negative control antibody (such as an isotype control) that may not bind MuSK. A preferred control antibody not binding to MuSK is Motavizumab which targets RSV (Review, MAbs, 1(5), 439-442, September-Octo 2009, DOI: 10.4161/mabs.1.5.9496). A preferred positive control agonist MuSK antibody is mAb #13 from Genentech. Another preferred positive control molecule for evidencing an activating MuSK activity is agrin (rat agrin from R&D systems, 550-AG).

The present invention provides exemplary MuSK agonist antibodies and antigen binding fragments thereof. These antibodies and antigen binding fragments serve as MuSK agonists in accordance with the invention. These antibodies and antigen binding fragments may be defined using the epitope they target and/or using their elicited activity as earlier defined. The exemplary MuSK agonist antibodies and antigen binding fragments of the invention may also be defined (preferably) exclusively with respect to their structural characteristics (sequence characteristics). The exemplary MuSK agonist antibodies and antigen binding fragments of the invention may also be defined using the epitope, their elicited activity and their structural characteristics. The structural characteristics of the agonist antibodies and antigen binding fragments of the invention are as described below.

In certain embodiments, the antibodies and antigen binding fragments that bind to MuSK or MuSK agonist antibodies are selected from antibody molecules comprising or consisting of a combination of a heavy chain variable domain (VH) and a light chain variable domain (VK) selected from the following:
(d1) a VH comprising the amino acid sequence of SEQ ID NO:37 or an amino acid sequence at least 90%, 95%, 97%, 98% or 99% identical thereto and a VK comprising the amino acid sequence of SEQ ID NO:57 or an amino acid sequence at least 90%, 95%, 97%, 98% or 99% identical thereto (3F6c);
(d2) a VH comprising the amino acid sequence of SEQ ID NO:36 or an amino acid sequence at least 90%, 95%, 97%, 98% or 99% identical thereto and a VK comprising the amino acid sequence of SEQ ID NO:56 or an amino acid sequence at least 90%, 95%, 97%, 98% or 99% identical thereto (3D10a); or
(d3) a VH comprising the amino acid sequence of SEQ ID NO:38 or an amino acid sequence at least 90%, 95%, 97%, 98% or 99% identical thereto and a VK comprising the amino acid sequence of SEQ ID NO:58 or an amino acid sequence at least 90%, 95%, 97%, 98% or 99% identical thereto (4D3).

Within the context of the invention, a VH or VK sequence may have less than 100% identity with a given sequence. This may means that the VH or VK sequence comprises one, two or three amino acid substitutions (e.g., conservative substitutions, humanising substitutions or affinity variants) in the recited sequence compared to the VH or VK sequence identified herein by a SEQ ID NO.

In an embodiment of this first aspect, there is provided a MuSK agonist antibody comprising:
A variable domain specifically binding to an epitope of MuSK;
A human antibody constant domain having an Fc domain which does not have complement effector functionality; and
a combination of a heavy chain variable domain (VH) and a light chain variable domain (VK) selected from the following:
   (d1) a VH comprising the amino acid sequence of SEQ ID NO:37 and a VK comprising the amino acid sequence of SEQ ID NO:57 (3F6c);
   (d2) a VH comprising the amino acid sequence of SEQ ID NO:36 and a VK comprising the amino acid sequence of SEQ ID NO:56 (3D10a); or
   (d3) a VH comprising the amino acid sequence of SEQ ID NO:38 and a VK comprising the amino acid sequence of SEQ ID NO:58 (4D3).

Such an agonistic MuSK antibody may exhibit at least one of: the induction of MuSK dimerization, the induction of MuSK tyrosine phosphorylation, the induction or increase of induction of acetylcholine receptor clustering at the NMJ (the clustering may be assessed in vitro in myotubes AChR patches), the increase of the number of fully innervated NMJ, the decrease of the number of fully denervated NMJ, an improvement of the reliability of synaptic transmission, a prevention/stabilization or even a reduction/decrease of motor neuron death, an extension of the lifespan of a treated subject. Each of these effects/activities have been earlier defined herein.

In an embodiment, the MuSK agonist antibody comprises a variable heavy chain domain (VH) and a variable light chain domain (VK) wherein the VH and VK domains comprise the CDR sequences selected from:
HCDR1 comprising or consisting of SEQ ID NO: 42; HCDR2 comprising or consisting of SEQ ID NO: 47; HCDR3 comprising or consisting of SEQ ID NO: 52; LCDR1 comprising or consisting of SEQ ID NO: 62; LCDR2 comprising or consisting of SEQ ID NO: 67; LCDR3 comprising or consisting of SEQ ID NO: 72 (3F6c);
HCDR1 comprising or consisting of SEQ ID NO: 41; HCDR2 comprising or consisting of SEQ ID NO: 46; HCDR3 comprising or consisting of SEQ ID NO: 51; LCDR1 comprising or consisting of SEQ ID NO: 61; LCDR2 comprising or consisting of SEQ ID NO: 66; LCDR3 comprising or consisting of SEQ ID NO: 71 (3D10a);
HCDR1 comprising or consisting of SEQ ID NO: 43; HCDR2 comprising or consisting of SEQ ID NO: 48; HCDR3 comprising or consisting of SEQ ID NO: 53; LCDR1 comprising or consisting of SEQ ID NO: 63; LCDR2 comprising or consisting of SEQ ID NO: 68; LCDR3 comprising or consisting of SEQ ID NO: 73 (4D3).

In an embodiment, this antibody has a variable domain binding to an epitope of MuSK. In an embodiment, the human antibody constant domain of this antibody has a Fc domain which does not complement effector functionality. Such an agonistic MuSK antibody may exhibit at least one of: the induction of MuSK dimerization, the induction of MuSK tyrosine phosphorylation, the induction or increase of induction of acetylcholine receptor clustering at the NMJ (or the clustering is assessed in vitro in myotubes AChR patches), the increase of the number of fully innervated NMJ, the decrease of the number of fully denervated NMJ, an improvement of the reliability of synaptic transmission, a prevention/stabilization or even a reduction/decrease of motor neuron death, an extension of the lifespan of a treated subject. Each of these effects/activities have been earlier defined herein.

In a preferred embodiment, this antibody exhibits an induction or increase of induction of acetylcholine receptor clustering at the NMJ assessed by staining for AChRs clustering at the NMJ of diaphragms of mice compared to the staining obtained without said antibody. In an embodiment, this induction or increase of clustering of AChRs at the NMJ results in a more normal/physiological NMJ morphology maintaining synaptic innervation and/or pre- and post-synaptic alignment. Each of this embodiment may be present alone or in combination in said antibody.

In certain embodiments, there is provided a MuSK agonist antibody or antigen binding fragment thereof, which binds MuSK, said antibody or antigen binding fragment comprising a heavy chain variable domain and a light chain variable domain, wherein
the variable heavy chain CDR1 sequence comprises or consists of SEQ ID NO: 42 or sequence variant thereof;
the variable heavy chain CDR2 sequence comprises or consists SEQ ID NO:47 or sequence variant thereof;
the variable heavy chain CDR3 sequence comprises or consists of SEQ ID NO:52 or sequence variant thereof;
the variable light chain CDR1 sequence comprises or consists of SEQ ID NO:62 sequence variant thereof;
the variable light chain CDR2 sequence comprises or consists SEQ ID NO:67 or sequence variant thereof;
the variable light chain CDR3 sequence comprises or consists of SEQ ID NO: 72 or sequence variant thereof; (3F6c) and
wherein the sequence variant comprises one, two or three amino acid substitutions (e.g., conservative substitutions, humanising substitutions or affinity variants) in the recited sequence.

In an embodiment, this antibody has a variable domain binding to an epitope of MuSK. In an embodiment, the human antibody constant domain of this antibody has a Fc domain which does not complement effector functionality. Such an agonistic MuSK antibody may exhibit at least one of: the induction of MuSK dimerization, the induction of MuSK tyrosine phosphorylation, the induction or increase of induction of acetylcholine receptor clustering at the NMJ (or the clustering is assessed in vitro in myotubes AChR patches), the increase of the number of fully innervated NMJ, the decrease of the number of fully denervated NMJ, an improvement of the reliability of synaptic transmission, a prevention/stabilization or even a reduction/decrease of motor neuron death, an extension of the lifespan of a treated subject. Each of these effects/activities have been earlier defined herein.

In a preferred embodiment, this antibody exhibits an induction or increase of induction of acetylcholine receptor clustering at the NMJ assessed by staining for AChRs clustering at the NMJ of diaphragms of mice compared to the staining obtained without said antibody. In an embodiment, this induction or increase of clustering of AChRs at the NMJ results in a more normal/physiological NMJ morphology maintaining synaptic innervation and/or pre- and post-synaptic alignment. Each of this embodiment may be present alone or in combination in said antibody.

In certain embodiments, there is provided a MuSK agonist antibody or antigen binding fragment thereof, which binds MuSK, said antibody or antigen binding fragment comprising a heavy chain variable domain and a light chain variable domain, wherein
the variable heavy chain CDR1 sequence comprises or consists of SEQ ID NO: 41 or sequence variant thereof;
the variable heavy chain CDR2 sequence comprises or consists SEQ ID NO:46 or sequence variant thereof;
the variable heavy chain CDR3 sequence comprises or consists of SEQ ID NO:51 or sequence variant thereof;
the variable light chain CDR1 sequence comprises or consists of SEQ ID NO:61 sequence variant thereof;
the variable light chain CDR2 sequence comprises or consists SEQ ID NO:66 or sequence variant thereof;
the variable light chain CDR3 sequence comprises or consists of SEQ ID NO: 71 or sequence variant thereof; (3D10a) and
wherein the sequence variant comprises one, two or three amino acid substitutions (e.g., conservative substitutions, humanising substitutions or affinity variants) in the recited sequence.

In an embodiment, this antibody has a variable domain binding to an epitope of MuSK. In an embodiment, the human antibody constant domain of this antibody has a Fc domain which does not complement effector functionality. Such an agonistic MuSK antibody may exhibit at least one of: the induction of MuSK dimerization, the induction of MuSK tyrosine phosphorylation, the induction or increase of induction of acetylcholine receptor clustering at the NMJ (or the clustering is assessed in vitro in myotubes AChR patches), the increase of the number of fully innervated NMJ, the decrease of the number of fully denervated NMJ, an improvement of the reliability of synaptic transmission, a prevention/stabilization or even a reduction/decrease of motor neuron death, an extension of the lifespan of a treated subject. Each of these effects/activities have been earlier defined herein.

In a preferred embodiment, this antibody exhibits an induction or increase of induction of acetylcholine receptor clustering at the NMJ assessed by staining for AChRs clustering at the NMJ of diaphragms of mice compared to the staining obtained without said antibody. In an embodiment, this induction or increase of clustering of AChRs at the NMJ results in a more normal/physiological NMJ morphology maintaining synaptic innervation and/or pre- and post-synaptic alignment. Each of this embodiment may be present alone or in combination in said antibody.

In certain embodiments, there is provided a MuSK agonist antibody or antigen binding fragment thereof, which binds MuSK, said antibody or antigen binding fragment comprising a heavy chain variable domain and a light chain variable domain, wherein the variable heavy chain CDR1 sequence comprises or consists of SEQ ID NO: 43 or sequence variant thereof;
the variable heavy chain CDR2 sequence comprises or consists SEQ ID NO:48 or sequence variant thereof;
the variable heavy chain CDR3 sequence comprises or consists of SEQ ID NO:53 or sequence variant thereof;
the variable light chain CDR1 sequence comprises or consists of SEQ ID NO:63 sequence variant thereof;
the variable light chain CDR2 sequence comprises or consists SEQ ID NO:68 or sequence variant thereof;
the variable light chain CDR3 sequence comprises or consists of SEQ ID NO: 73 or sequence variant thereof; (4D3) and
wherein the sequence variant comprises one, two or three amino acid substitutions (e.g., conservative substitutions, humanising substitutions or affinity variants) in the recited sequence.

In an embodiment, this antibody has a variable domain binding to an epitope of MuSK. In an embodiment, the human antibody constant domain of this antibody has a Fc domain which does not complement effector functionality. Such an agonistic MuSK antibody may exhibit at least one of: the induction of MuSK dimerization, the induction of MuSK tyrosine phosphorylation, the induction or increase of induction of acetylcholine receptor clustering at the NMJ (or the clustering is assessed in vitro in myotubes AChR patches), the increase of the number of fully innervated NMJ, the decrease of the number of fully denervated NMJ, an improvement of the reliability of synapse release, a prevention/stabilization or even a reduction/decrease of motor neuron death, an extension of the lifespan of a treated subject. Each of these effects/activities have been earlier defined herein.

In a preferred embodiment, this antibody exhibits an induction or increase of induction of acetylcholine receptor clustering at the NMJ assessed by staining for AChRs clustering at the NMJ of diaphragms of mice compared to the staining obtained without said antibody. In an embodiment, this induction or increase of clustering of AChRs at the NMJ results in a more normal/physiological NMJ morphology maintaining synaptic innervation and/or pre- and post-synaptic alignment. Each of this embodiment may be present alone or in combination in said antibody.

In an embodiment, the antibody comprises a variable heavy chain domain (VH) and a variable light chain domain (VK) wherein the VH and VK domains comprise the CDR sequences selected from:
a VH comprising the amino acid sequence of SEQ ID NO:37 or an amino acid sequence at least 90%, 95%, 97%, 98% or 99% identical thereto and a VK comprising the amino acid sequence of SEQ ID NO:57 or an amino acid sequence at least 90%, 95%, 97%, 98% or 99% identical thereto (3F6c) and wherein the HCDR1 comprising or consisting of SEQ ID NO: 42; the HCDR2 comprising or consisting of SEQ ID NO: 47; the HCDR3 comprising or consisting of SEQ ID NO: 52; the LCDR1 comprising or consisting of SEQ ID NO: 62; the LCDR2 comprising or consisting of SEQ ID NO: 67; and the LCDR3 comprising or consisting of SEQ ID NO: 72 (3F6c);
a VH comprising the amino acid sequence of SEQ ID NO:36 or an amino acid sequence at least 90%, 95%, 97%, 98% or 99% identical thereto and a VK comprising the amino acid sequence of SEQ ID NO:56 or an amino acid sequence at least 90%, 95%, 97%, 98% or 99% identical thereto (3D10a); and wherein the HCDR1 comprising or consisting of SEQ ID NO: 41; the HCDR2 comprising or consisting of SEQ ID NO: 46; the HCDR3 comprising or consisting of SEQ ID NO: 51; the LCDR1 comprising or consisting of SEQ ID NO: 61; LCDR2 comprising or consisting of SEQ ID NO: 66; and the LCDR3 comprising or consisting of SEQ ID NO: 71 (3D10a);
a VH comprising the amino acid sequence of SEQ ID NO:38 or an amino acid sequence at least 90%, 95%, 97%, 98% or 99% identical thereto and a VK comprising the amino acid sequence of SEQ ID NO:58 or an amino acid sequence at least 90%, 95%, 97%, 98% or 99% identical thereto (4D3); and wherein the HCDR1 comprising or consisting of SEQ ID NO: 43; the HCDR2 comprising or consisting of SEQ ID NO: 48; the HCDR3 comprising or consisting of SEQ ID NO: 53; the LCDR1 comprising or consisting of SEQ ID NO: 63; the LCDR2 comprising or consisting of SEQ ID NO: 68; and the LCDR3 comprising or consisting of SEQ ID NO: 73 (4D3).

In an embodiment, this antibody has a variable domain binding to an epitope of MuSK. In an embodiment, the human antibody constant domain of this antibody has a Fc domain which does not complement effector functionality. Such an agonistic MuSK antibody may exhibit at least one of: the induction of MuSK dimerization, the induction of MuSK tyrosine phosphorylation, the induction or increase of induction of acetylcholine receptor clustering at the NMJ (or the clustering is assessed in vitro in myotubes AChR patches), the increase of the number of fully innervated NMJ, the decrease of the number of fully denervated NMJ, an improvement of the reliability of synapse release, a prevention/stabilization or even a reduction/decrease of motor neuron death, an extension of the lifespan of a treated subject. Each of these effects/activities have been earlier defined herein.

In a preferred embodiment, this antibody exhibits an induction or increase of induction of acetylcholine receptor clustering at the NMJ assessed by staining for AChRs clustering at the NMJ of diaphragms of mice compared to the staining obtained without said antibody. In an embodiment, this induction or increase of clustering of AChRs at the NMJ results in a more normal/physiological NMJ morphology maintaining synaptic innervation and/or pre- and post-synaptic alignment. Each of this embodiment may be present alone or in combination in said antibody.

In an embodiment, the antibody comprises a heavy chain and a light chain as identified below:
a heavy chain comprising or consisting of the amino acid sequence of SEQ ID NO:131 or an amino acid sequence at least 90%, 95%, 97%, 98% or 99% identical thereto and a light chain comprising the amino acid sequence of SEQ ID NO:132 or an amino acid sequence at least 90%, 95%, 97%, 98% or 99% identical thereto (3F6c);
a heavy chain comprising or consisting of the amino acid sequence of SEQ ID NO:127 or an amino acid sequence at least 90%, 95%, 97%, 98% or 99% identical thereto and a light chain comprising the amino acid sequence of SEQ ID NO:128 or an amino acid sequence at least 90%, 95%, 97%, 98% or 99% identical thereto (3D10a);
a heavy chain comprising or consisting of the amino acid sequence of SEQ ID NO:129 or an amino acid sequence at least 90%, 95%, 97%, 98% or 99% identical thereto and a light chain comprising the amino acid sequence of SEQ ID NO:130 or an amino acid sequence at least 90%, 95%, 97%, 98% or 99% identical thereto (4D3).

In an embodiment, this antibody has a variable domain binding to an epitope of MuSK. In an embodiment, the human antibody constant domain of this antibody has a Fc domain which does not complement effector functionality. Such an agonistic MuSK antibody may exhibit at least one of: the induction of MuSK dimerization, the induction of MuSK tyrosine phosphorylation, the induction or increase of induction of acetylcholine receptor clustering at the NMJ (or the clustering is assessed in vitro in myotubes AChR patches), the increase of the number of fully innervated NMJ, the decrease of the number of fully denervated NMJ, an improvement of the reliability of synapse release, a prevention/stabilization or even a reduction/decrease of motor neuron death, an extension of the lifespan of a treated subject. Each of these effects/activities have been earlier defined herein.

In a preferred embodiment, this antibody exhibits an induction or increase of induction of acetylcholine receptor clustering at the NMJ assessed by staining for AChRs clustering at the NMJ of diaphragms of mice compared to the staining obtained without said antibody. In an embodiment, this induction or increase of clustering of AChRs at the NMJ results in a more normal/physiological NMJ morphology maintaining synaptic innervation and/or pre- and post-synaptic alignment. Each of this embodiment may be present alone or in combination in said antibody.

In an embodiment, the antibody of the invention does not comprise or does not consist of the heavy chain and light chain as identified below:
a heavy chain comprising or consisting of the amino acid sequence of SEQ ID NO:125 and a light chain comprising the amino acid sequence of SEQ ID NO:126 (3B5);
a heavy chain comprising or consisting of the amino acid sequence of SEQ ID NO: 133 and a light chain comprising the amino acid sequence of SEQ ID NO: 134 (3D9b).

For embodiments wherein the domains of the antibodies or antigen binding fragments are defined by a particular percentage sequence identity to a reference sequence, the VH and/or VL domains may retain identical CDR sequences to those present in the reference sequence such that the variation is present only within the framework regions (FR1, FR2, FR3, FR4 of the VK or of the VL domain are identified as SEQ ID NO: 85 to 124) A framework region variant encompassed by the invention may comprise one, two or three amino acid substitutions (e.g., conservative substitutions, humanising substitutions or affinity variants) by comparison to the recited sequence in any of SEQ ID NO: 85 to 124.

In certain embodiments, there is provided a MuSK agonist antibody or antigen binding fragment thereof, which binds MuSK, said antibody or antigen binding fragment comprising a heavy chain variable domain and a light chain variable domain, wherein
the variable heavy (VH) chain FR1 sequence comprises or consists of SEQ ID NO: 86, 87 or 88 or sequence variant thereof;
the variable heavy (VH) chain FR2 sequence comprises or consists SEQ ID NO:91, 92 or 93 or sequence variant thereof;
the variable heavy (VH) chain FR3 sequence comprises or consists of SEQ ID NO:96, 97 or 98 or sequence variant thereof;
the variable heavy (VH) chain FR4 sequence comprises or consists of SEQ ID NO:101, 102 or 103 or sequence variant thereof;
the variable light (VL) chain FR1 sequence comprises or consists of SEQ ID NO:106, 107 or 108 sequence variant thereof;
the variable light (VL) chain FR2 sequence comprises or consists SEQ ID NO:111, 112 or 113 or sequence variant thereof;
the variable light (VL) chain FR3 sequence comprises or consists of SEQ ID NO: 116, 117 or 118 or sequence variant thereof; and/or the variable light (VL) chain FR4 sequence comprises or consists of SEQ ID NO: 121, 122 or 123 or sequence variant thereof;
wherein the sequence variant comprises one, two or three amino acid substitutions (e.g., conservative substitutions, humanising substitutions or affinity variants) in the recited sequence.

The invention also provides antibodies or antigen binding fragments thereof, which bind to the same epitope as the MuSK antibodies exemplified herein. The identification and further characterization of such antibodies or antigen binding fragment can be done using a competition assay as defined earlier herein.

In certain embodiments, the exemplary MuSK agonist antibodies and antigen binding fragments thereof defined as having the CDR sequences recited above or defined as having a particular percentage identity to the specific VH/VL domain amino acid sequences recited above are humanised, germlined or affinity variants or are considered to be derived from, camelid derived or are considered the antibodies or antigen binding fragments thereof from which the CDR, VH and/or VL sequences derive.

In a preferred embodiment, the exemplary MuSK antibody having the CDR sequences recited above exhibit high human homology, for example are humanised or germlined variants of the antibodies or antigen binding fragments thereof from which the CDR sequences derive. Such MuSK agonist antibody may be considered to be substantially human as earlier defined herein.

In non-limiting embodiments, the exemplary MuSK antibodies and antigen binding fragments thereof having the CDR, VH and/or VL (or VK) sequences described herein may comprise CH1 domains and/or CL domains (from the heavy chain and light chain, respectively), the amino acid sequence of which is fully or substantially human. Such substantially human antibodies have been defined earlier herein. For antibody molecules intended for human therapeutic use, it is typical for the entire constant region of the antibody, or at least a part thereof, to have fully or substantially human amino acid sequence. Therefore, one or more or any combination of the CH1 domain, hinge region, CH2 domain, CH3 domain and CL domain (and CH4 domain if present) may be fully or substantially human with respect to its amino acid sequence.

Advantageously, the CH1 domain, hinge region, CH2 domain, CH3 domain and CL domain (and CH4 domain if present) may all have fully or substantially human amino acid sequence or are considered to be derived from a human corresponding sequence. All these terms have been defined earlier herein. In the context of the constant region of a humanised or chimeric antibody, or an antibody fragment, the term “substantially human” refers to an amino acid sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence, excepting those embodiments where the presence of a “fully human” hinge region is expressly required. Any of the exemplary Fc region modifications described herein may be incorporated into the MuSK antibodies having the CDR and/or VH/VL domain sequences recited above. In certain embodiments, the MuSK antibodies having the CDR and/or VH/VL domain sequences recited above comprise a modified human IgG Fc domain comprising or consisting of the amino acid substitutions H433K and N434F, wherein the Fc domain numbering is in accordance with EU numbering. In certain embodiments, the MuSK antibodies having the CDR and/or VH/VL domain sequences recited above comprise a modified human IgG Fc domain comprising or consisting of the amino acid substitutions M252Y, S254T, T256E, H433K and N434F.

Unless otherwise stated in the present application, % sequence identity between two amino acid sequences (usually each identified by a SEQ ID NO) may be determined by comparing these two sequences aligned in an optimum manner and in which the amino acid sequence to be compared can comprise additions or deletions with respect to the reference sequence for an optimum alignment between these two sequences. In an embodiment, the alignment is carried out over the full length of one of two amino acid sequences. However, it is also encompassed that the alignment is carried out over part of the length of one of the two amino acid sequences. In this context, “part” means 50%, 55%, 60%, 65%, 70%, 75%, 80%, 85%, 90%, 95%, 99% of the length of one of the two amino acid sequences. The percentage of identity is calculated by determining the number of identical positions for which the amino acid residue is identical between the two sequences, dividing this number of identical positions by the total number of positions in the comparison window and multiplying the result obtained by 100 in order to obtain the percentage of identity between these two sequences.

For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/gorf/bl2.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

In preferred embodiments, the MuSK agonists of the present invention are antibodies or antigen binding fragments thereof. The term “antibody” herein is used in the broadest sense and encompasses, but is not limited to, monoclonal antibodies (including full length monoclonal antibodies), polyclonal antibodies, multispecific antibodies (e.g., bispecific antibodies), so long as they exhibit the appropriate immunological specificity and agonistic activity for the MuSK protein. The MuSK agonist antibodies and antigen binding fragments described herein may exhibit immunological specificity for any of the MuSK epitopes described in section B above.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen.

“Antibody fragments” or “antigen binding fragments” comprise a portion of a full length antibody, generally the antigen binding or variable domain thereof. Antibody fragments are described elsewhere herein and examples of antibody fragments include Fab, Fab′, F(ab′)2, bi-specific Fab's, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-36 (2005), the contents of which are incorporated herein by reference).

The MuSK agonist antibodies and antigen binding fragments described herein are intended for human therapeutic use and therefore, will typically be immunoglobulins of the IgA, IgD, IgE, IgG, IgM type, often of the IgG type, in which case they can belong to any of the four sub-classes IgG1, IgG2a and b, IgG3 or IgG4. In preferred embodiments, the MuSK agonist antibodies are IgG antibodies. Monoclonal antibodies are preferred since they are highly specific, being directed against a single antigenic site.

The MuSK agonist antibodies and antigen binding fragments thereof may exhibit high human homology as defined elsewhere herein. Such antibody molecules having high human homology may include antibodies comprising VH and VL domains of native non-human antibodies which exhibit sufficiently high percent sequence identity to human germline sequences. In this context, exhibiting a “sufficiently high percent identity” means exhibiting a sequence identity or sequence homology of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the antibodies or antigen binding fragments thereof are humanised or germlined variants of non-human antibodies.

In certain embodiments, the MuSK agonist antibodies and antigen binding fragments described herein may be camelid-derived. Camelid-derived antibodies may be heavy-chain only antibodies i.e. VHH antibodies or may be conventional heterotetrameric antibodies. In preferred embodiments, the MuSK agonist antibodies and antigen binding fragments are derived from camelid heterotetrameric antibodies.

For example, the MuSK agonist antibodies and antigen binding fragments may be selected from immune libraries obtained by a method comprising the step of immunizing a camelid with the target of interest i.e. MuSK. The camelid may be immunized with the target protein or polypeptide fragment thereof, or with an mRNA molecule or cDNA molecule expressing the protein or a polypeptide fragment thereof. Methods for producing antibodies in camelid species and selecting antibodies against preferred targets from camelid immune libraries are described in, for example, International patent application no. WO2010/001251, incorporated herein by reference.

In certain embodiments, the MuSK agonist antibodies and antigen binding fragments may be camelid-derived in that they comprise at least one hypervariable (HV) loop or complementarity determining region obtained from a VH domain or a VL domain of a species in the family Camelidae. In particular, the MuSK agonist antibodies and antigen binding fragments may comprise VH and/or VL domains, or CDRs thereof, obtained by active immunisation of outbred camelids, e.g. llamas, with MuSK agonist.

The term “obtained from” in this context implies a structural relationship, in the sense that the HVs or CDRs of the antibodies embody an amino acid sequence (or minor variants thereof) which was originally encoded by a Camelidae immunoglobulin gene. However, this does not necessarily imply a particular relationship in terms of the production process used to prepare the antibodies or antigen binding fragments thereof.

Camelid-derived antibodies or antigen binding fragments thereof may be derived from any camelid species, including inter alia, llama, dromedary, alpaca, vicuna, guanaco or camel.

Antibody molecules comprising camelid-derived VH and VL domains, or CDRs thereof, are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously. Included within this definition are “species” chimeric polypeptides created by juxtaposition of peptide fragments encoded by two or more species, e.g. camelid and human.

In certain embodiments, the entire VH domain and/or the entire VL domain may be obtained from a species in the family Camelidae. The camelid-derived VH domain and/or the camelid-derived VL domain may then be subject to protein engineering, in which one or more amino acid substitutions, insertions or deletions are introduced into the camelid amino acid sequence. These engineered changes preferably include amino acid substitutions relative to the camelid sequence. Such changes include “humanisation” or “germlining” wherein one or more amino acid residues in a camelid-encoded VH or VL domain are replaced with equivalent residues from a homologous human-encoded VH or VL domain.

Isolated camelid VH and VL domains obtained by active immunisation of a camelid (e.g. llama) with MuSK can be used as a basis for engineering MuSK agonist antibodies and antigen binding fragments in accordance with the present invention. Starting from intact camelid VH and VL domains, it is possible to engineer one or more amino acid substitutions, insertions or deletions which depart from the starting camelid sequence. In certain embodiments, such substitutions, insertions or deletions may be present in the framework regions of the VH domain and/or the VL domain.

In other embodiments, there are provided “chimeric” antibody molecules comprising camelid-derived VH and VL domains (or engineered variants thereof) and one or more constant domains from a non-camelid antibody, for example human-encoded constant domains (or engineered variants thereof). In such embodiments it is preferred that both the VH domain and the VL domain are obtained from the same species of camelid, for example both VH and VL may be from Lama glama or both VH and VL may be from Lama pacos (prior to introduction of engineered amino acid sequence variation). In such embodiments both the VH and the VL domain may be derived from a single animal, particularly a single animal which has been actively immunised with the antigen of interest.

As an alternative to engineering changes in the primary amino acid sequence of Camelidae VH and/or VL domains, individual camelid-derived hypervariable loops or CDRs, or combinations thereof, can be isolated from camelid VH/VL domains and transferred to an alternative (i.e. non-Camelidae) framework, e.g. a human VH/VL framework, by CDR grafting.

In non-limiting embodiments, the MuSK agonist antibodies may comprise CH1 domains and/or CL domains (from the heavy chain and light chain, respectively), the amino acid sequence of which is fully or substantially human. The expression “substantially human” has been earlier defined herein. For antibody molecules intended for human therapeutic use, it is typical for the entire constant region of the antibody, or at least a part thereof, to have fully or substantially human amino acid sequence. Therefore, one or more or any combination of the CH1 domain, hinge region, CH2 domain, CH3 domain and CL domain (and CH4 domain if present) may be fully or substantially human with respect to its amino acid sequence. The CH1 domain, hinge region, CH2 domain, CH3 domain and/or CL domain (and/or CH4 domain if present) may be derived from a human antibody, preferably a human IgG antibody, more preferably a human IgG1 antibody of subtype IgG1, IgG2, IgG3 or IgG4.

Advantageously, the CH1 domain, hinge region, CH2 domain, CH3 domain and CL domain (and CH4 domain if present) may all have fully or substantially human amino acid sequence. The term “substantially human” in the context of an antibody of the invention or part thereof has already been defined herein. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence, excepting those embodiments where the presence of a “fully human” hinge region is expressly required.

The MuSK agonist antibodies may have one or more amino acid substitutions, insertions or deletions within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites).

The MuSK agonist antibodies may be modified within the Fc region to increase or decrease binding affinity for the neonatal receptor FcRn. The increased binding affinity may be measurable at acidic pH (for example from about approximately pH 5.5 to approximately pH 6.0). The increased binding affinity may also be measurable at neutral pH (for example from approximately pH 6.9 to approximately pH 7.4). By “increased binding affinity” is meant increased binding affinity to FcRn relative to the unmodified Fc region. A standardized pH gradient FcRn affinity liquid chromatography method may be used as known to the skilled person (such as the one described in mAntibodies, 2013, 5:576-586). Typically the unmodified Fc region will possess the wild-type amino acid sequence of human IgG1, IgG2, IgG3 or IgG4. In such embodiments, the increased FcRn binding affinity of the antibody molecule having the modified Fc region will be measured relative to the binding affinity of wild-type IgG1, IgG2, IgG3 or IgG4 for FcRn.

In an embodiment, the human antibody constant domain of the MuSK agonist antibody of the invention is a human IgG4 constant domain.

In certain embodiments, one or more amino acid residues within the Fc region may be substituted with a different amino acid so as to increase binding to FcRn. Several Fc substitutions have been reported that increase FcRn binding and thereby improve antibody pharmacokinetics. Such substitutions are reported in, for example, Zalevsky et al. (2010) Nat. Biotechnol. 28(2):157-9; Hinton et al. (2006) J Immunol. 176:346-356; Yeung et al. (2009) J Immunol. 182:7663-7671; Presta L G. (2008) Curr. Op. Immunol. 20:460-470; and Vaccaro et al. (2005) Nat. Biotechnol. 23(10):1283-88, the contents of which are incorporated herein in their entirety.

In certain embodiments, the MuSK agonist antibodies comprise a modified human IgG Fc domain comprising or consisting of the amino acid substitutions H433K and N434F, wherein the Fc domain numbering is in accordance with EU numbering. In a further embodiment, the MuSK agonist antibodies described herein comprise a modified human IgG Fc domain comprising or consisting of the amino acid substitutions M252Y, S254T, T256E, H433K and N434F, wherein the Fc domain numbering is in accordance with EU numbering.

In certain embodiments, the MuSK agonist antibodies comprise a modified human IgG Fc domain consisting of up to 2, up to 3, up to 4, up to 5, up to 6, up to 7, up to 8, up to 9, up to 10, up to 12, up to 15, up to 20 substitutions relative to the corresponding wild-type, control, reference, naturally occurring IgG sequence.

In certain embodiments, the human antibody constant domain of the MuSK agonist antibody has an Fc domain which does not have complement effector functionality.

In particular embodiments, the Fc region may be engineered such that there is no or reduced effector function. In certain embodiments, the MuSK agonist antibody of the invention may have an Fc region derived from naturally-occurring IgG isotypes having reduced effector function, for example IgG4. Fc regions derived from IgG4 may be further modified to increase therapeutic utility, for example by the introduction of modifications that minimise the exchange of arms between IgG4 molecules in vivo. Fc regions derived from IgG4 may be modified to include the S228P substitution. Fc receptor binding can be assessed according to methods known in the art, including for example testing binding of an antibody to Fc receptor protein in a BIACORE assay.

In an embodiment, the human antibody constant domain of the MuSK agonist antibody is derived from a human antibody which naturally has complement effector functionality, and which contains a mutation that silences the complement effector functionality. In an embodiment, the human antibody constant domain is derived from human IgG1, human IgG2, or human IgG3. In another embodiment, the human antibody constant domain is derived from human IgG1. In an embodiment, the mutations in the human antibody constant domain comprise L234A and L235A.

In certain embodiments, the antibody molecules are modified with respect to glycosylation. For example, an aglycoslated antibody can be made (i.e., the antibody lacks glycosylation). Glycosylation can be altered to, for example, increase the affinity of the antibody for the target antigen. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen.

### C. Polynucleotides Encoding MuSK Agonist Antibodies

The invention also provides polynucleotide molecules encoding the antibodies of the invention or fragments thereof. Polynucleotide molecules encoding the full-length antibodies are provided, together with polynucleotide molecules encoding fragments, for example the VH, VL (or VK), CDR3 domains of the antibodies described herein. These polynucleotide molecules are represented by nucleotide sequences.

In an embodiment of this aspect, there is provided a polynucleotide as defined above, wherein the nucleotide sequence encodes an antibody of the invention comprising a combination of a heavy chain variable domain (VH) and a light chain variable domain (VK) selected from the following:
(d1) a nucleotide sequence comprising or consisting of SEQ ID NO: 27 or a nucleotide sequence at least 90%, 95%, 97%, 98% or 99% identical thereto, preferably said nucleotide sequence encoding a VH comprising the amino acid sequence of SEQ ID NO:37 or an amino acid sequence at least 90%, 95%, 97%, 98% or 99% identical thereto and a nucleotide sequence comprising or consisting of SEQ ID NO: 32 or a nucleotide sequence at least 90%, 95%, 97%, 98% or 99% identical thereto, preferably said nucleotide sequence encoding VK comprising the amino acid sequence of SEQ ID NO:57 or an amino acid sequence at least 90%, 95%, 97%, 98% or 99% identical thereto (3F6c);
(d2) a nucleotide sequence comprising or consisting of SEQ ID NO: 26 or a nucleotide sequence at least 90%, 95%, 97%, 98% or 99% identical thereto, preferably said nucleotide sequence encoding VH comprising the amino acid sequence of SEQ ID NO:36 or an amino acid sequence at least 90%, 95%, 97%, 98% or 99% identical thereto and a nucleotide sequence comprising or consisting of SEQ ID NO: 31 or a nucleotide sequence at least 90%, 95%, 97%, 98% or 99% identical thereto, preferably said nucleotide sequence encoding a VK comprising the amino acid sequence of SEQ ID NO:56 or an amino acid sequence at least 90%, 95%, 97%, 98% or 99% identical thereto (3D10a); or
(d3) a nucleotide sequence comprising or consisting of SEQ ID NO: 28 or a nucleotide sequence at least 90%, 95%, 97%, 98% or 99% identical thereto, preferably said nucleotide sequence encoding a VH comprising the amino acid sequence of SEQ ID NO:38 or an amino acid sequence at least 90%, 95%, 97%, 98% or 99% identical thereto and
a nucleotide sequence comprising or consisting of SEQ ID NO: 33 or a nucleotide sequence at least 90%, 95%, 97%, 98% or 99% identical thereto, preferably said nucleotide sequence encoding a VK comprising the amino acid sequence of SEQ ID NO:58 or an amino acid sequence at least 90%, 95%, 97%, 98% or 99% identical thereto (4D3);

In an embodiment, this antibody encoded by this polynucleotide sequence has a variable domain binding to an epitope of MuSK. In an embodiment, the human antibody constant domain of this antibody encoded by this polynucleotide sequence has a Fc domain which does not complement effector functionality. Such an agonistic MuSK antibody encoded by such a polynucleotide may exhibit at least one of: the induction of MuSK dimerization, the induction of MuSK tyrosine phosphorylation, the induction or increase of induction of AChR clustering at the NMJ (or the clustering is assessed in vitro in myotubes AChR patches), the increase of the number of fully innervated NMJ, the decrease of the number of fully denervated NMJ, an improvement of the reliability of synapse release, a prevention/stabilization or even a reduction/decrease of motor neuron death, an extension of the lifespan of a treated subject. Each of these effects/activities have been earlier defined herein.

In a preferred embodiment, this antibody encoded by this polynucleotide sequence exhibits an induction or increase of induction of AChR clustering at the NMJ assessed by staining for AChRs clustering at the NMJ of diaphragms of mice compared to the staining obtained without said antibody. In an embodiment, this induction or increase of clustering of AChRs at the NMJ results in a more normal/physiological NMJ morphology maintaining synaptic innervation and/or pre- and post-synaptic alignment. Each of this embodiment may be present alone or in combination in said antibody.

Also provided are expression vectors containing said nucleotide sequences of the invention. In an embodiment, said expression vectors further comprise a regulatory sequence. Such a regulatory sequence is operably linked to the nucleotide sequences of the invention, which permit expression of the antibodies or fragments thereof in a host cell or cell-free expression system. The invention also relates to such a host cell or cell-free expression system containing this expression vector.

Polynucleotide molecules encoding MuSK agonist antibodies of the invention include, for example, recombinant DNA molecules. The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). An isolated polynucleotide (either DNA or RNA) may further represent a molecule produced directly by biological or synthetic means and separated from other components present during its production. In an embodiment, a nucleic acid or polynucleotide is considered to have bene “engineered” as it has been obtained using recombinant DNA technology and it does not exist as such in the nature

For recombinant production of a MuSK agonist antibody according to the invention, a recombinant polynucleotide encoding it or recombinant polynucleotides encoding the different chains or domains may be prepared (using standard molecular biology techniques) and inserted into a replicable vector for expression in a chosen host cell, or a cell-free expression system. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); mouse myeloma cells SP2/0-AG14 (ATCC CRL 1581; ATCC CRL 8287) or NSO (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art.

It should be noted that the term “host cell” generally refers to a cultured cell line. Whole human beings into which an expression vector encoding an antigen binding polypeptide according to the invention has been introduced are explicitly excluded from the definition of a “host cell”. In an embodiment, a cell or host cell is considered to have bene “engineered” as it has been obtained using recombinant DNA technology and it does not exist as such in the nature.

### D. Antibody Production

In a further aspect, the invention also provides a method of producing antibodies of the invention which comprises culturing a host cell (or cell free expression system) containing polynucleotide (e.g. an expression vector) encoding the antibody under conditions which permit expression of the antibody, and recovering the expressed antibody. This recombinant expression process can be used for large scale production of antibodies, including MuSK agonist antibodies according to the invention, including monoclonal antibodies intended for human therapeutic use. Suitable vectors, cell lines and production processes for large scale manufacture of recombinant antibodies suitable for in vivo therapeutic use are generally available in the art and will be well known to the skilled person.

### E. Compositions/Pharmaceutical Compositions

In a further aspect, the invention relates to a composition comprising a MuSK agonist antibody, a polynucleotide, an expression vector or a host cell or cell-free expression system as defined in earlier sections of the description.

In an embodiment, the composition is a pharmaceutical composition. In an embodiment, a pharmaceutical composition comprises a MuSK agonist antibody, a polynucleotide, an expression vector or a host cell or cell-free expression system as defined in earlier sections of the description and at least one pharmaceutically acceptable carrier or excipient. In a preferred embodiment, the pharmaceutical composition comprises a MuSK agonist antibody or antigen binding fragments thereof, formulated with one or more pharmaceutically acceptable carriers or excipients. The preparation of a pharmaceutical composition that contains at least one active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference).

Such composition may include one or a combination of (e.g., two or more different) MuSK agonist antibodies of the invention. Techniques for formulating monoclonal antibodies for human therapeutic use are well known in the art and are reviewed, for example, in Wang et al., Journal of Pharmaceutical Sciences, Vol. 96, pp 1-26, 2007, the contents of which are incorporated herein in their entirety.

Pharmaceutically acceptable excipients that may be used to formulate the compositions include, but are not limited to: ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In certain embodiments, the compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration.

### F. Methods of Treatment

In a further aspect, the invention relates to a method of treating a subject in need thereof, wherein the method comprises administering to the subject a therapeutically effective amount of an antibody, a polynucleotide, an expression vector, a host cell or cell-free expression system, a pharmaceutical composition. Each of a MuSK agonist antibody, a polynucleotide, an expression vector, a host cell or cell-free expression system and a pharmaceutical composition has been extensively defined in earlier sections of the description.

In a further aspect, the invention relates to a MuSK agonist antibody, a polynucleotide, an expression vector, a host cell or cell-free expression system, a pharmaceutical composition for use as a medicament. Each of a MuSK agonist antibody, a polynucleotide, an expression vector, a host cell or cell-free expression system and a pharmaceutical composition has been extensively defined in earlier sections of the description.

In an embodiment, a MuSK agonist, particularly the MuSK agonist antibodies and antigen binding fragments described herein, may be used in a method of treatment. Thus, provided herein is a MuSK agonist antibody in accordance with the first aspect of the invention for use as a medicament. Alternatively, provided herein is a MuSK agonist antibody in accordance with the first aspect of the invention for use in a method of treatment. In preferred embodiments, the invention provides MuSK antibodies and antigen binding fragments as described elsewhere herein for use as medicaments. Alternatively, the invention provides MuSK antibodies and antigen binding fragments as described elsewhere herein for use in a method of treatment. The MuSK agonists, including the MuSK agonist antibodies and antigen binding fragments thereof, for use as medicaments are typically formulated as pharmaceutical compositions. Importantly, all embodiments described above in relation to MuSK agonists, particularly the MuSK agonist antibodies and antigen binding fragments thereof, are equally applicable to the methods described herein.

The present invention also provides methods of treating a subject in need thereof, wherein the method comprises administering to the subject a therapeutically effective amount of a MuSK agonist antibody, a polynucleotide, an expression vector, a host cell or cell-free expression system or a pharmaceutical composition. Each of a MuSK agonist antibody, a polynucleotide, an expression vector, a host cell or cell-free expression system and a pharmaceutical composition has been extensively defined in earlier sections of the description. In such methods of treatment, the MuSK agonists, including the MuSK agonist antibodies and antigen binding fragments thereof, are typically formulated as pharmaceutical compositions.

In an embodiment, the subject is treated for a disease or condition associated with an impaired neuromuscular transmission. The activation of MuSK is expected to transduce signal at the NMJ and therefore to restore at least to some extent an impaired neuromuscular transmission. The MuSK agonist antibody and related aspects of the invention (i.e. polynucleotide, expression vector, host cell, cell-free expression system as earlier defined herein) are said to restore at least to some extent an impaired neuromuscular transmission when they are able to elicit an agonistic MuSK activity. In the context of the application, an agonistic MuSK activity may be replaced by the triggering of a MuSK-induced signal in a muscle cell at the NMJ. A MuSK-induced signal may be at least one of the induction of MuSK dimerization, the induction of MuSK tyrosine phosphorylation, the induction of AChR clustering at the NMJ (or the clustering is assessed in vitro in myotubes AChR patches), the increase of the number of fully innervated NMJ, the decrease of the number of fully denervated NMJ, an improvement of the reliability of synapse release, a prevention/stabilization or even a reduction/decrease of motor neuron death, an extension of the lifespan of a treated subject.

Accordingly in an embodiment, there is provided an antibody, a polynucleotide, an expression vector, a host cell or cell-free expression system, a pharmaceutical composition for use for treating a disease or condition associated with an impaired neuromuscular transmission. In an embodiment, the disease or condition associated with an impaired neuromuscular transmission is a neurodegenerative disease, preferably Amyotrophic Lateral Sclerosis (ALS), Alzheimer's disease, Huntington's disease, Parkinson's disease, frontotemporal dementia, Spinal Muscular Atrophy (SMA), congenital myasthenia, Emery-Dreifuss muscular dystrophy, Charcot-Marie tooth, Myasthenia Gravis, (post)polyomyelitis. A condition associated with an impaired neuromuscular transmission may be aging or age-related muscle wasting, sarcopenia. Accordingly in another embodiment, the disease or condition treated in the method of treatment of the invention is one of the diseases or conditions listed above.

In a further aspect, the invention relates to the use of a MuSK agonist antibody, a polynucleotide, an expression vector, a host cell or cell-free expression system, a pharmaceutical composition for the manufacture of a medicament for treating a disease or condition associated with an impaired neuromuscular transmission. Each of a MuSK agonist antibody, a polynucleotide, an expression vector, a host cell or cell-free expression system and a pharmaceutical composition has been extensively defined in earlier sections of the description. In an embodiment, the disease or condition associated with an impaired neuromuscular transmission is a neurodegenerative disease, preferably Amyotrophic Lateral Sclerosis (ALS), Alzheimer's disease, Huntington's disease, Parkinson's disease, frontotemporal dementia, Spinal Muscular Atrophy (SMA), congenital myasthenia, Emery-Dreifuss muscular dystrophy, Charcot-Marie tooth, Myasthenia Gravis, (post)polyomyelitis. A condition associated with an impaired neuromuscular transmission may be aging or age-related muscle wasting, sarcopenia.

As used herein, the term “therapeutically effective amount” is intended to mean the quantity or dose of MuSK agonist, e.g. antibody, that is sufficient to produce a therapeutic effect, for example, the quantity or dose of agonist required to elicit an MuSK agonistic activity. In an embodiment, such agonistic MuSK activity is able to eradicate or at least alleviate the symptoms associated with a disease or condition associated with an impaired neuromuscular transmission. An appropriate amount or dose can be determined by a physician, as appropriate.

As used herein, a method of “preventing” a disease or condition means preventing the onset of the disease, preventing the worsening of symptoms, preventing the progression of the disease or condition or reducing the risk of a subject developing the disease or condition. As used herein, a method of “treating” a disease or condition means curing a disease or condition and/or alleviating or eradicating the symptoms associated with the disease or condition such that the patient's suffering is reduced.

### G. Kits

Any of the MuSK agonists, antibodies or antigen binding fragments described herein can be packaged as a kit and optionally include instructions for use.

### General

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” when used in association with an integer (about 10) preferably means that the value may be the given value of 10 more or less 1 of the value: about 10 preferably means from 9 to 11. The word “about” when used in association with a numerical value (about 10.6) preferably means that the value may be the given value of 10.6 more or less 1% of the value 10.6.

All patent and literature references cited in the present specification are hereby incorporated by reference in their entirety. The following examples are offered for illustrative purposes only, and are not intended to limit the scope of the present invention in any way.

### Examples

The invention will be further understood with reference to the following non-limiting examples.

### Experimental Procedures

### Antibodies

Antibodies were produced at small scale in HEK293 cells at argenx and at large scale by U-protein Express (UPX) using the hIgG1-LALA-delK backbone. The references for the batch numbers, reports and relevant information about the antibodies is summarized in the tables 1 and 2. All antibodies were formulated in PBS with or without 0.02% Tween80. Endotoxin levels were determined at Eurofins.

TABLE 1
information about the antibodies used in this
report-small scale productions at argenx

| | 3D10a | 4D3 | 3F6c | 3D9b | mAb13 | Motavizumab |
|---|---|---|---|---|---|---|
| Batch nr | 20190503 | 20190503 | 20190503 | 20190503 | 20190903 | na |
| | | | | 20190813 | 20190920 | |

TABLE 2
information about the antibodies used in this report-large scale production at UPX

| | 3D10a | 4D3 | 3F6c | 3D9b | mAb13 | Motavizumab |
|---|---|---|---|---|---|---|
| UPX report reference | 2019-230 | | | | 2019-258 | 2019-259 |
| Batch nr | 3832 | 3833 | 3834 | 3835 | 3905 | 3906 |
| Human identity (%) | 99.4 | 100 | 100 | 100 | 96.3 | Na |
| Production yield (mg/L) HEK293 | 204 | 156 | 224 | 263 | 223 | 133 |
| Concentration (mg/mL) | 7.3 | 7.3 | 7.1 | 6.3 | 4.4 | 6.0 |
| Aggregates HPLC (%) | <1% | <1% | <1% | <1% | <1% | <1% |
| Endotoxin (EU/mg) | 0.0068 | 0.069 | 0.07 | 0.008 | 0.01 | 0.003 |

### Off-Target Binding

A Nunc Maxisorp plate was coated with 1% baculovirus particles overnight at 4° C. The next day, the plate was washed with PBS and blocked with 0.5% BSA faction V—PBS (Sigma) and incubated for 1 hour at room temperature (RT). Plate was washed with PBS. The samples were diluted to a concentration of 1.5, 15 and 150 pg/ml in blocking buffer and applied to the wells of a non-coated (but blocked) plate and to the baculovirus coated wells and allowed to bind for one hour at RT. The plate was washed with PBS. Next, detecting antibody was added and incubated for one hour at RT (Goat anti-human IgG (Fcγ fragment specific), HRP conjugated) in blocking buffer. Lastly, the plate was washed with PBS and TMB was added and the reaction was stopped with 0.5 M H2S04. OD 450 nm was determined by Elisa reader. The ratio of signal obtained on the coated well is divided by the signal obtained in the non-coated well which leads to an off-target score.

As controls, Lampalizumab (low off target binding) and 169C8 (high off target binding) were included.

### Thermostability

For this purpose, the antibodies were treated with a gradient of an increasing temperature from 55° C. up to 75° C. with Thermocycler (Biometra). Next, their binding capacity was analysed on Biacore 3000 to CM5 Chip coated with human Musk (R&D systems, Lot nr DHUCO 11908A, batch nr 1089_MK). Raw data were analyzed via BIA evaluation software with a blank subtraction. The percentage of activity was plotted in GraphPad Prism (Log (agonist) vs response, variable slope (4 parameters). The temperature where the antibody has lost 50% of its binding capacity was reported as TM50. Experiment 1 and 2 were done using a Biacore method where MuSK was coated at low density (2000 RU). Experiment 3 was done using a Biacore method where MuSK was coated at high density (6000 RU).

### Temperature Stress Testing

This study was done blinded. All samples were filtered using a 0.2 μm filter. The antibodies were diluted to 5 mg/mL in PBS containing 0.02% Tween80 and stored in glass vials at 4° C. and or 37° C. for a maximum of 6 weeks. Every week, samples were analysed for visual inspection. A negative control consisting of buffer only and 1 high aggregation control was included (mAb 36C4). Samples were analysed for integrity by Capillary electrophoresis SDS (CE-SDS), activity in Biacore (using the method described in the paragraph entitled “Thermostability” and using a calibration curve), protein content in nanodrop, aggregate formation in SE-HPLC and chemical modifications by masspectrometry (RPC-MS).

### Freeze Thaw Stability

This study was done blinded. All samples were filtered using a 0.2 μm filter. The antibodies were diluted to 5 mg/mL in PBS containing 0.02% Tween80 in glass vials. Samples underwent in total five cycles of freeze/thawing: antibodies were frozen at −20° C. for at least 6 hours and then thawed at RT for 1 hour. Samples were analysed for activity in Biacore (using the method described in the paragraph entitled “Thermostability” and using a calibration curve) and for aggregate formation in SE-HPLC.

### Binding to Mouse Neuromuscular Junctions in Immunohistochemistry

Mouse levator auris longus muscle (10 per NOD/SCID mouse) were dissected and immunostained with 1 μg of agonistic anti-MuSK antibody. The NMJs/AChRs were marked using BTX594 and bound monoclonals were detected with an anti-human IgG alexa fluor 488 antibody. Imaging was performed under an epifluorescent microscope with varying settings to get the best picture. All MuSK antibodies clearly label synaptic regions (colocalization with BTX). This method was also described in Neurology: Neuroimmunology & Neuroinflammation, 6: 1-9, 2019.

### Binding Affinity for Mouse, Human and Rhesus Monkey Recombinant MuSK in Biacore

Sequence of mouse and rhesus monkey MuSK was derived from the UNIPROT database. The genes were made by Geneart and cloned in the PUPE vector for expression in HEK293 cells (at argenx).

The MuSK variants contained a streptavidine and Flag tag, the latter was used for purification. Human MuSK was obtained from R&D Systems (produced in CHO cells, cat. 10189-MK-MTO, lot DHUC011905A) and contains a histidine tag.

For affinity determination, an anti-hFC (Jackson #109-005-098) CM5 chip (8000RU) was used that captures the antibodies of interest (1 fixed concentration of 1 μg/mL). Next human, rhesus or mouse MuSK was injected at different concentrations (0.3125-0.625-1.25-2.5-5-10 nM) to allow the determination of the affinity (KD).

### pH Dependency for Binding to MuSK in Biacore

For affinity determination, an anti-hFC (Jackson #109-005-098) CM5 chip (8000RU) was used that captures the antibodies of interest (1 fixed concentration of 1 μg/mL). Next human MuSK was injected at different concentrations (0.3125-0.625-1.25-2.5-5-10 nM) to allow the determination of the affinity (KD).

Two conditions were tested:

Association of MuSK in HBS EP pH 7.4 and dissociation in HBS EP pH 7.4 buffer Association of MuSK in HBS EP pH 7.4 and dissociation in HBS EP pH 5.5 buffer

Ca²⁺ and Mg²⁺ dependent binding to MuSK using mesoscale

A mesoscale (MSD) plate was coated with 1 μg/mL human monomeric MuSK-6×his (R&D Systems, produced in CHO cells, cat. 10189-MK-MTO, lot DHUC011905A), diluted in 1×PBS and incubate overnight at 4° C. Next, the plated was washed 3 times with 1×TBS pH7.4 (0.05% Tween 20), and blocked with 1% BSA in 1×TBS pH7.4 for 2h at RT, shaking at 600 rmp. The plated was wash 3 times with 1×TBS pH7.4. Next, antibodies were added starting at a concentration of 2 μg/ml. Fivefold dilutions were made (7 point in total) in 1×TBS pH7.4, 1×TBS 3 mM Ca2+ pH7.4, 1×TBS 1 mM Mg2+ pH7.4. Anti-human Fc SULFO tag (cat. #R32AJ-1, MesoScale Discovery, Rockville, Md., USA) was used as detection antibody.

### Epitope Mapping in ELISA: Binding to MuSK Domains and Epitope Binning

For binding to MuSK domains—An ELISA plate was coated with 0.5 μg/ml of anti-FLAG (M2 clone, Sigma Cat. F3165) and incubated overnight at 4° C. Next, the plate was washed with PBS and blocked with 1×PBS 1% casein for two hours at RT. After washing the plate, human monomeric MuSK FLAG streptavidin (batch 27-6-2019) and truncated MuSK variants (batch 3-9-2019) at 1 μg/ml in PBS where incubated for one hour at RT. After washing the plate, antibodies were diluted in blocking buffer and added to the plate. After a one hour incubation at RT, the plate was washed. Next, detecting antibody was added and incubated for one hour at RT (anti-human Fc HRP (1/5000) (clone 5A9, cat. No. Ab7499, ABCAM)) in blocking buffer. Lastly, the plate was washed with PBS and TMB was added and the reaction was stopped with 0.5 M H2SO4. OD 450 nm was determined by the Tecan Sunrise ELISA plate reader.

For epitope binning—An ELISA plate was coated at 1 μg/ml of the anti-MuSK Fabs and incubated overnight at 4° C. Next, the plate was wash with PBS followed by blocking with 1% casein in PBS for one hour. After washing the plate, 1 μg/ml of human monomeric MuSK (in house produced, batch 27-6-2019) was incubated for one hour at RT. The plate was washed and anti-MuSK antibodies at 0.3-10 μg/ml were added to the plate and incubated for one hour at RT. After washing the plate, detection antibody (goat anti-human Fc HRP (Jackson Immunoresearch cat. 109-035-098)) was added at 5000 fold dilution in PBS 0.1% casein. The plate was incubated for one hour at RT and washed with PBS-T. TMB was added and the reaction was stopped by adding H2504. OD at 250 nm was measured with the Tecan Sunrise ELISA plate reader.

### Epitope Mapping in Biacore

A chip was coated with ˜5000 RU of human MuSK (Lot nr DHUCO 11908A, batch nr 1089_MK). In a first experiment the saturating concentration of the pre-leads was determined. This is the concentration where the amplitude of a specific pre-lead doesn't increase anymore when additional pre-lead is injected on the immobilized MuSK.

For the epitope mapping experiment, a saturating concentration of each antibody was injected (this is the ‘first antibody’) and at the end of the injection time a second antibody was injected, to check if it still can bind to MuSK when MuSK is pre-saturated with the first antibody. After regeneration of the chip, the second antibody was injected alone (without pre-saturating MuSK with the first antibody) to monitor the amplitude of this antibody.

### Phosphorylation Assay

To determine the ability of the antibodies to induce MuSK phosphorylation we exposed C2C12 myotubes cultures (Cell lines service) to the antibodies as described previously (Huijbers & Zhang et al 2013 PNAS). Differentiated myotubes were stimulated with 0.1 nM agrin (550-AG-100, R&D systems) in the presence of antibodies (100 ng/mL). Immunoprecipitation of MuSK was initiated after 30 minutes of exposure: the myotubes were lysed and MuSK was precipitated using 1 mg/sample hIgG1 anti MuSK (clone 11-3F6 LUMC production) during an overnight incubation at 4° C. (Huijbers & Zhang et al. 2013 PNAS). Bound antigen-antibody complexes were precipitated using protein A agarose beads (11134515001, Roche) which were extensively washed and dissolved in 1× Laemmli sample buffer. Samples were subsequently ran on SDS-PAGE gel and transferred to PDVF membrane. MuSK and phosphorylated MuSK was detected using goat anti-rat MuSK (1:2000, AF562, R&D systems) and mouse anti-phosphotyrosine clone 4G10 (1:1000, 05-321, Millipore) as primary antibodies, and donkey anti-mouse-680RD (1:10,000, 926-68072, Licor) and donkey anti-goat 800CW (1:10,000, 926-32214, Licor) as secondary antibodies. Bound antibodies were detected using the Odyssey CLx (Licor).

### AChR Clustering Assay

AChR clustering was studied after 16 hours of exposing C2C12 derived myotubes to either 110 ng/mL recombinant antibodies or 0.1 nM agrin in a ELISA plate (655090, Greiner). In a separate experiment a dose response of the antibodies was tested (0.11, 1.1, 11 and 110 μg/mL). Subsequently the cells were washed three times with differentiation medium (DMEM, 31966 Gibco, 2% heat inactivated horse serum 26050-088, Gibco, 1% pen/strep and 1% L-glutamine) and incubated with 5 μg/mL AlexaFluor488 conjugated α-bungarotoxin (B13422, ThermoFisher) in differentiation medium for 30 minutes at 37° C. Nuclei were visualized using Hoechst. After staining, cells were fixed in 4% formalin solution for 10 minutes, washed three times with PBS and kept in the last wash at 4° C. for a maximum of one week before imaging. Every independent experiment consisted of five wells per condition and a minimum of four fields per well were randomly selected and imaged with Leica AF6000 microscope. AChR cluster count was analysed using Fiji (2.0.0v).

### AChR Clustering Assay in the Presence of MG Patient Derived Polyclonal Antibodies

AChR clustering was studied after 24 hours. First, C2C12 derived myotubes where exposed to polyclonal IgG from a patient for 1 hour, thereafter 110 ng/ml recombinant antibodies or 0.1 nM agrin was added and incubated for a total of 24 hours. Subsequently the cells were washed three times with differentiation medium (DMEM, 31966 Gibco, 2% heat inactivated horse serum 26050-088, Gibco, 1% pen/strep and 1% L-glutamine) and incubated with, 5 μg/mL AlexaFluor488 conjugated α-bungarotoxin (B13422, ThermoFisher) in differentiation medium for 30 minutes at 37° C. Nuclei were visualized using Hoechst. After staining cells were fixed in 4% formalin solution for 10 minutes, washed three times with PBS and kept in the last wash at 4° C. for a maximum of one week before imaging. Every independent experiment consisted of five wells per condition and four fields per well were randomly selected and imaged with Leica AF6000 microscope. AChR cluster count was analysed using Fiji (2.0.0v).

TABLE 3
Antibodies disclosed belonging to the invention
(SEQ ID NO as identified in the sequence listing)

| | VH aa | | | | VK aa | | | |
|---|---|---|---|---|---|---|---|---|
| | (DNA) | HCDR1 | HCDR2 | HCDR3 | (DNA) | LCDR1 | LCDR2 | LCDR3 |
| 3F6c | 37 (27) | 42 | 47 | 52 | 57 (32) | 62 | 67 | 72 |
| 3D10a | 36 (26) | 41 | 46 | 51 | 56 (31) | 61 | 66 | 71 |
| 4D3 | 38 (28) | 43 | 48 | 53 | 58 (33) | 63 | 68 | 73 |

The heavy chain of 3F6c is represented by SEQ ID NO: 131. The light chain of 3F6c is represented by SEQ ID NO: 132.

The heavy chain of 3D10a is represented by SEQ ID NO: 127. The light chain of 3D10a is represented by SEQ ID NO: 128.

The heavy chain of 4D3 is represented by SEQ ID NO: 129. The light chain of 4D3 is represented by SEQ ID NO: 130.

TABLE 4
Antibodies disclosed but not belonging to the invention
(SEQ ID NO as identified in the sequence listing)

| | VH aa | | | | VK aa | | | |
|---|---|---|---|---|---|---|---|---|
| | (DNA) | HCDR1 | HCDR2 | HCDR3 | (DNA) | LCDR1 | LCDR2 | LCDR3 |
| 3D9b | 35 (25) | 40 | 45 | 50 | 55 (30) | 60 | 65 | 70 |
| 3B5 | 39 (29) | 44 | 49 | 54 | 59 (34) | 64 | 69 | 74 |

The heavy chain of 3B5 is represented by SEQ ID NO: 125. The light chain of 3B5 is represented by SEQ ID NO: 126.

The heavy chain of 3D9b is represented by SEQ ID NO: 133. The light chain of 3D9b is represented by SEQ ID NO: 134.

### Example 1: Off Target Binding

A majority of human therapeutic antibody candidates show pharmacokinetic properties suitable for clinical use, but an unexpectedly fast antibody clearance is sometimes observed that may limit the clinical utility. Off target binding can contribute to unwanted fast clearance. An Elisa method was described (mAntibodies 2012, 4: 753-760) in which detection of non-specific binding to baculovirus particles was shown to correlate with antibodies having increased risk for fast clearance.

Table 5 summarizes the signal obtained in the ELISA for the different antibodies tested at 100 versus 10 μg/mL, including lampalizumab (control 1), an antibody with low off target binding, and 169C6 (control 2) with high off target binding properties. The highest signal is obtained for 3D9b, with OD≥2.3 on all wells tested, indicating high off target binding for this antibody. In general, all antibodies demonstrate high off target binding as compared to mAb13 and the low control 1.

TABLE 5
OD in ELISA for antibodies binding to baculovirus
lysates (baculo) versus non coated well (blanc)

| OD | Control 1 Low | Control 2 High | 3D10a | 4D3 | 3F6c | 3D9b | mAb13 |
|---|---|---|---|---|---|---|---|
| Baculo - 100 μg/mL | 0.2 | 3.1 | 2.7 | 2.9 | 4.1 | 3.9 | 2.4 |
| Baculo - 10 μg/mL | 0.1 | 1.4 | 0.9 | 0.9 | 0.8 | 2.3 | 0.4 |
| Blanc - 100 μg/mL | 0.05 | 0.2 | 1.2 | 1.4 | 0.5 | 3.7 | 1.7 |
| Blanc - 10 μg/mL | 0.06 | 0.02 | 0.2 | 0.3 | 0.1 | 2.6 | 0.3 |

### Example 2: Thermostability

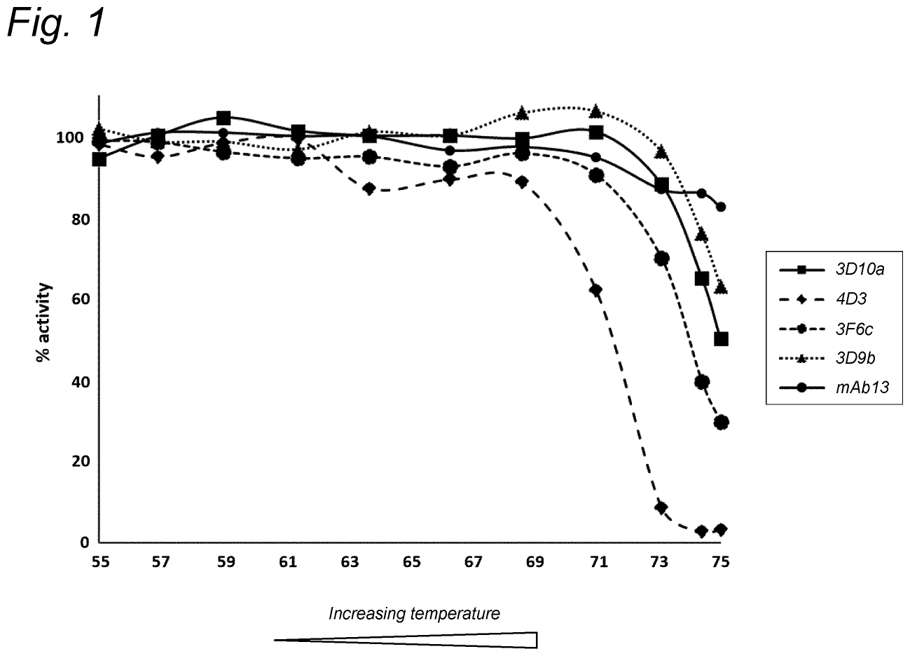

Antibodies were incubated at increasing temperature and then analysed for remaining biological activity in order to calculate the Tm50. Two independent experiments were performed. Binding to MuSK was tested in Biacore using 2 different coating densities: method 1 uses a low coating density whereas method 2 uses a higher coating density of MuSK. The conclusion is that all antibodies are extremely stable with Tm>71° C. (FIG. 1).

TABLE 6
Summary table for the thermostability testing from 2 independent
experiments using 2 different Biacore methods.

| Tm50 (° C.) | 3D10a | 4D3 | 3F6c | 3D9b | mAb13 |
|---|---|---|---|---|---|
| Experiment 1 - method 1 | >75 | 73 | >75 | ambiguous | ND |
| Experiment 2 - method 1 | ~73 | 72 | ~75 | ambiguous | ND |
| Experiment 2 - method 2 | ~75 | 71 | ~74 | ~74 | ~77 |

### Example 3: Temperature Stress Testing

Antibodies were diluted to 5 mg/mL in PBS containing 0.02% Tween80 and stored in glass vials at 4° C. and or 37° C. for a maximum of 6 weeks.

Every week, samples were analyzed for visual inspection. A negative control consisting of buffer only and 1 high aggregation control was included (mAb 36C4). Table 7 demonstrates that for all antibodies after visual inspection no aggregates were noted by 4 independent and blinded observers.

TABLE 7
summary visual inspection on aggregate formation after incubation
for 6 weeks at 4° C. or 37° C.

| | 3D10a | 4D3 | 3F6c | 3D9b | mAb13 |
|---|---|---|---|---|---|
| 6 weeks at 4° C. | A/A/A | A/A/A | A/A/A | Af/A/A | Na |
| 6 weeks at 37° C. | Af/B/Af | A/A/A | Af/B/Af | A/A/Af | Na |

A: sample is clear, no particles visible;
B: very few particles;
C: moderate presence of particles;
D: abundant particles observed;
f: fibers

Samples were analyzed for integrity by Capillary electrophoresis SDS (CE-SDS) and for protein content in nanodrop, no obvious degradation could be observed.

Aggregate formation was analyzed using SE-HPLC. Results in Table 8 indicate that aggregate levels are all below 1.5% and therefore we can conclude that no aggregates are formed after 6 weeks incubation at 37° C.

TABLE 8
SE-HPLC data on high molecular weight aggregate formation after
incubation for 6 weeks at 4° C. or 37° C.

| Aggregates (%) | 3D10a | 4D3 | 3F6c | 3D9b | mAb13 |
|---|---|---|---|---|---|
| 6 weeks at 4° C. | 0.1 | 0.1 | 0.1 | 1.4 | Na |
| 6 weeks at 37° C. | 0.3 | 0.6 | 0.4 | 0.3 | Na |

Next, sample were tested for activity in Biacore (using the method described in the paragraph “Thermostability” and using a calibration curve to allow calculating % activity). For all antibodies tested, activity remains 90-114% after 6 weeks incubation at 37° C. indicating that no major changes in the CDRs are expected.

TABLE 9
binding to MuSK in Biacore (expressed as percentage activity
versus a standard curve setting the time zero sample at
100%) after incubation for 6 weeks at 4° C. or 37° C.

| Rel. potency (%) | 3D10a | 4D3 | 3F6c | 3D9b | mAb13 |
|---|---|---|---|---|---|
| 6 weeks at 4° C. | 121 | 107 | 119 | 99 | Na |
| 6 weeks at 37° C. | 108 | 114 | 90 | 104 | Na |

During the stress test, samples were taken for analysis by LC-MS based peptide mapping at aRIC. A separate report is available about these results, but the findings are also summarized in the Table 10 below.

TABLE 10
chemical degradation as tested by LC-MS based peptide mapping
after 6 weeks incubation at 37° C. in the VH and VL (results
for constant regions are not included in this summary table)

| | | | | | | |
|---|---|---|---|---|---|---|
| Deamidation | HC (077-087) | FRWK3 | 3.6 | 2.9 | 4.2 | 4.6 |
| | LC (001-018) | FWK1 | 0.7 | 0.8 | 0.9 | 1.2 |
| | HC (099-107) | CDR3 | NA | NA | NA | 0.7 |
| | LC (046-061) | FWK2-CDR2 | NA | NA | NA | 3.9 |
| | LC (029-042) | CDR1-FWK1 | 0.3 | NA | NA | NA |
| Isomerization | HC (044-065) | FWK2-CDR2 | 2.0 | 2.1 | NA | NA |
| | HC (104-125) | CDR3-FWK4 | 0.3 | NA | NA | NA |
| Oxidation | LC (001-018) | FWK1 | 0.2 | 0.2 | 0.3 | 0.2 |

### Example 4: Freeze-Thaw Stability

In this study we evaluated if our antibodies are stable after repeated freeze thaw cycles. Table 11 and 13 demonstrate that indeed antibodies are stable after 5 cycles of freezing and thawing as we saw no major loss of activity in Biacore (89-120%, using same method as in 7.4) and aggregate levels were <1%.

TABLE 11
binding to MuSK in Biacore (expressed as percentage
activity versus a standard curve setting the time
zero sample at 100%) after 5 freeze - thaw cycles.

| Rel. potency (%) | 3D10a | 4D3 | 3F6c | 3D9b | mAb13 |
|---|---|---|---|---|---|
| 5x Freeze/thaw | 120 | 89 | 101 | 96 | Na |

TABLE 12
SE-HPLC data on high molecular weight aggregate
formation after 5 freeze - thaw cycles.

| Aggregates (%) | 3D10a | 4D3 | 3F6c | 3D9b | mAb13 |
|---|---|---|---|---|---|
| 5x Freeze/thaw | 0.1 | 0.1 | 0.1 | <0.1 | Na |

### Example 5: Binding to Mouse Neuromuscular Junctions in Immunohistochemistry

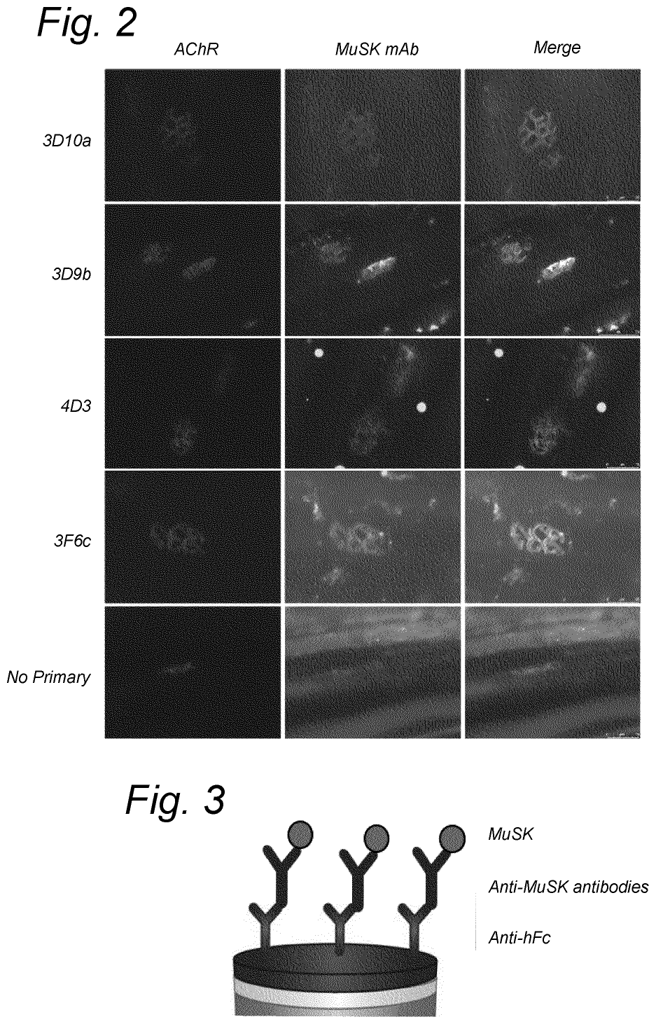

Demonstrating binding of our antibodies to mouse neuromuscular junctions (NMJs) is essential since we want to test these antibodies in mouse efficacy models. The results demonstrate that all of the pre-lead antibodies can bind to the characteristic “pretzel-like” structure of the mouse NMJ outlined by α-BTX (FIG. 2). This confirms ex-vivo binding of our antibodies to the mouse NMJ.

### Example 6: Binding Affinity for Mouse, Human and Rhesus Monkey Recombinant MuSK in Biacore

Animal cross-reactivity of candidate leads was analysed to assess which animal models can be used for in vivo proof of concept studies and which species is most suited for toxicology studies. At this stage we only investigated human, rhesus monkey and mouse, other species will be done later. The alignment of the sequence of human, cynomolgus monkey, rhesus monkey and mouse MuSK is shown below and shows an identity with human between 91-99% (Table 13).

TABLE 13
% identity and % similarity compared to full
length human MuSK extracellular domain

| | % identity | % similarity |
|---|---|---|
| Cynomolgus | 98.9 | 99.6 |
| Rhesus | 98.7 | 99.6 |
| Mouse | 91.3 | 97.2 |
| Rat | 91.5 | 97.7 |

TABLE 14
Amino acid positions of the different domains within
the extracellular domain of MuSK for each species.

| | Ig-like 1 | Ig-like 2 | Ig-like 3 | Frizzled |
|---|---|---|---|---|
| | domain | domain | domain | domain |
| Human MuSK | 28-116 | 121-205 | 212-302 | 312-450 |
| SEQ ID NO: 1 | | | | |
| Cynomolgus | 28-116 | 121-205 | 212-302 | 312-450 |
| MuSK | | | | |
| SEQ ID NO: 80 | | | | |
| Rhesus MuSK | 28-116 | 121-205 | 212-302 | 312-450 |
| SEQ ID NO: 82 | | | | |
| Mouse MuSK | 28-116 | 121-205 | 212-302 | 312-450 |
| SEQ ID NO: 83 | | | | |
| Rat MuSK | 28-116 | 121-205 | 212-302 | 312-450 |
| SEQ ID NO: 84 | | | | |

Ig-like 1 domain
1-LPKAPVITTPLETVDALVEEVATFMCAVESYPQPEISWTRNKILIKLFDTRYSIRENGQLLTILSVEDSDDGIYCCTANNGVGGAVESCGALQVKMKP
1-LPKAPVITTPLETVDALVEEVATFMCAVESYPQPEISWTRNKILIKLFDTRYSIRENGQLLTILSVEDSDDGIYCCTANNGVGGAVESCGALQVKMKP
1-LPKAPVITTPLETVDALVEEVATFMCAVESYPQPEISWTRNKILIKLFDTRYSIRENGQLLTILSVEDSDDGIYCCTANNGVGGAVESCGALQVKMKP

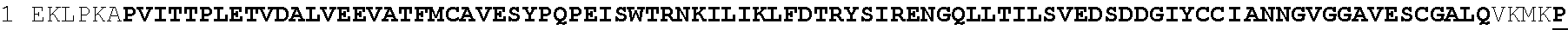

1 EKLPKAPVITTPLETVDALVEEVATFMCAVESYPQPEISWTRNKILIKLFDTRYSIRENGQLLTILSVEDSDDGIYCCTANNGVGGAVESCGALQVKMKP
Ig-like 2 domain
99
KITRPPINVKIIEGLKAVLPCTTMGNPKPSVSWIKGDSPLRENSRIAVLESGSLRIHNVOKEDAGOYRCVAKNSLGTAYSKVVKLEVEVFARILRAPESH
99

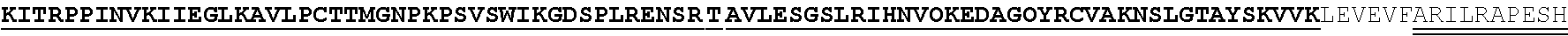

99

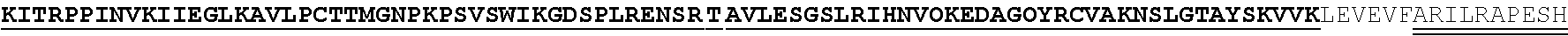

101

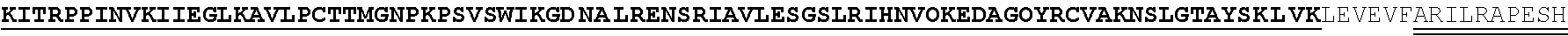

101

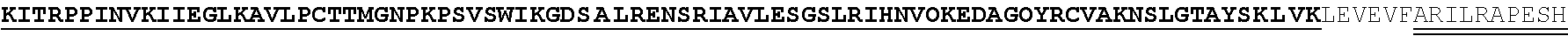

Ig-like 3 domain
199

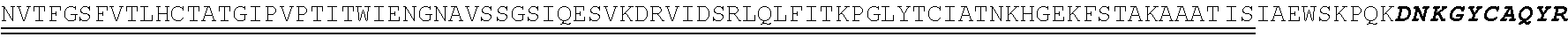

199

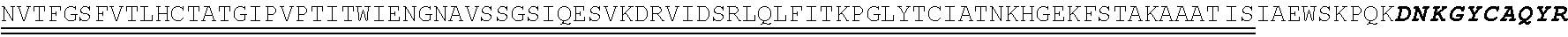

199
NVTFGSFVTLHCTATGIPVPTITWIENGNAVSSGSIQESVKDRVIDSRLOLFITKPGLYTCIATNKHGEKFSTAKAAATVSIAEWSKPQKDNKGYCAQYR
201

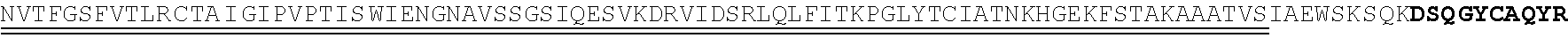

201

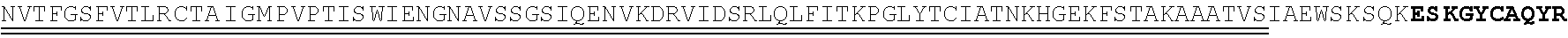

Frizzled domain
299

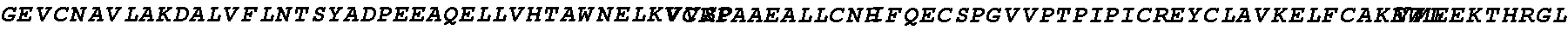

299

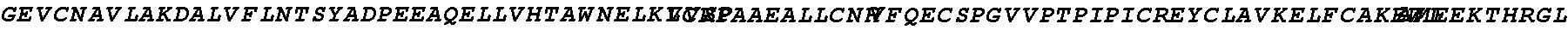

299

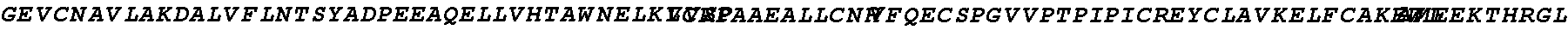

301

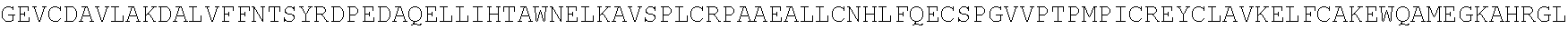

301

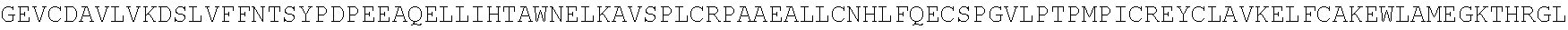

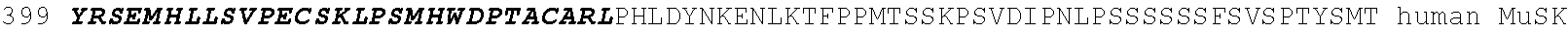

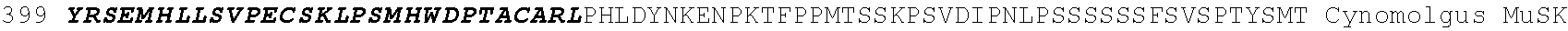

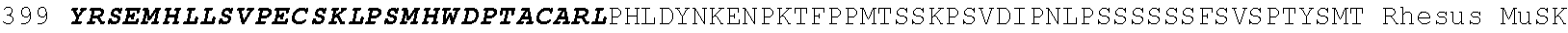

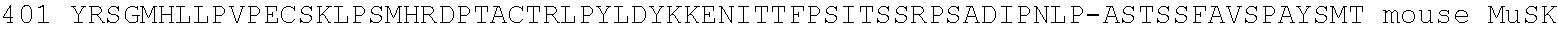

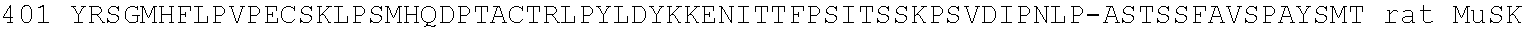

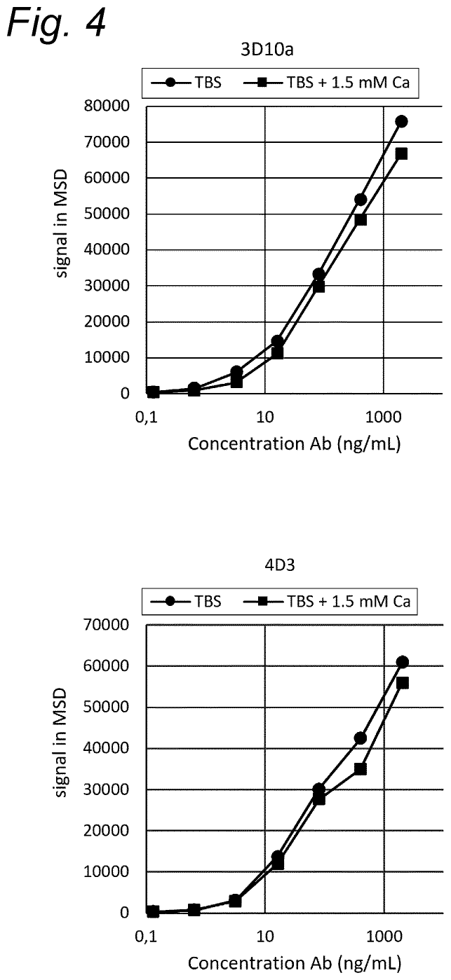

The affinity of 3D10a, 4D3, 3F6c and 3D9b for human, rhesus monkey and mouse MuSK was tested in Biacore using the setup shown in FIG. 3. Affinities were very comparable for all the antibodies for the three species, and within a factor 10 difference (Table 15). The affinities are very high keeping in mind that MuSK was added here in solution, allowing only for monovalent binding (affinity versus avidity). The affinity of the antibodies is ≤0.3 nM. For mAb13 the affinity for mouse MuSK is clearly lower (3 nM), whereas the affinity for human and rhesus monkey MuSK is comparable.

TABLE 15
Table summarizing the affinity (KD) of antibodies
for human, mouse and rhesus monkey MuSK.

| KD (nM) | 3D10a | 4D3 | 3F6c | 3D9b | mAb13 |
|---|---|---|---|---|---|
| Human | 0.16 | 0.26 | 0.05 | 0.17 | 0.24 |
| Rhesus monkey | 0.21 | 0.31 | 0.13 | 0.28 | 0.08 |
| Mouse | 0.10 | 0.17 | 0.03 | 0.14 | 3.19 |

### Example 7: pH Dependency for Binding to MuSK in Biacore

Conventional antibodies are taken up into cells by non-specific endocytosis or pinocytosis or via receptor mediated internalization. A Recycling Antibody is engineered so that a single antibody molecule can bind to an antigen multiple times, in contrast to conventional antibodies, which can only bind antigen once. Indeed, once conventional antibodies bind to a membrane anchored antigen such as a receptor, the antibody-antigen complex is internalized and degraded within the lysosome. This results in a shorter plasma half-life of the therapeutic antibody, necessitating frequent administration of the antibody drug or at higher doses to sustain efficacious plasma antibody concentration.

Antibodies can be engineered such that the antibody dissociates from the antigen at acidic pH within the endosome. Once dissociated, the recycling antibody is free to bind to the FcRn (neonatal Fc Receptor) within the endosome, which transports the antibody back into circulation to bind to more antigen

Most of the pH-dependent antibodies reported so far have been obtained after heavy engineering of the CDRs but sometimes this property can be pre-existing. We investigated in Biacore if one of our antibodies has the naturally existing pH dependency for binding to MuSK.

In order to study pH dependent binding of our antibodies to MuSK, the Biacore was used with the same setup as described in the paragraph entitled “Binding affinity for mouse, human and rhesus monkey recombinant MuSK in Biacore”. 6. The results are summarized in Table 16-18 and demonstrate that 3F6c binds with some pH dependency to MuSK, with a decreased affinity at pH 5.5. 3D9b binds also with some pH dependency to MuSK, but with a 3 fold increased affinity at pH 5.5 which could potentially lead to a shorter half life in vivo. For the other antibodies there is no real difference observed between pH 7.4 and 5.5.

TABLE 16
off rates (koff) in Biacore for binding of different concentrations
(0.3-10 nM) of antibodies to MuSK at neutral pH (pH 7.4).

| koff (10-4/s) | 3D10a | 4D3 | 3F6c | 3D9b |
|---|---|---|---|---|
| 0.3125 nM | 1.8 | 1.3 | 1.5 | 1.8 |
| 0.625 nM | 1.3 | 1.6 | 0.4 | 1.4 |
| 1.25 nM | 1.6 | 1.5 | 0.7 | 1.4 |
| 2.5 nM | 1.8 | 1.5 | 0.6 | 0.9 |
| 5 nM | 1.5 | 1.4 | 0.7 | 0.8 |
| 10 nM | 1.4 | 1.3 | 0.6 | 0.8 |
| Average | 1.6 | 1.4 | 0.8 | 1.2 |
| STD | 0.2 | 0.1 | 0.4 | 0.4 |

TABLE 17
off rates (koff) in Biacore for binding of different concentrations
(0.3-10 nM) of antibodies to MuSK at endosomal pH (pH 5.5).

| koff (10-4/s) | 3D10a | 4D3 | 3F6c | 3D9b |
|---|---|---|---|---|
| 0.3125 nM | 0.6 | 1.8 | 2.3 | 0.3 |
| 0.625 nM | 1.2 | 1.3 | 1.4 | 0.4 |
| 1.25 nM | 0.7 | 1.6 | 1.8 | 0.3 |
| 2.5 nM | 0.9 | 1.4 | 2.0 | 0.4 |
| 5 nM | 1.0 | 1.8 | 2.0 | 0.4 |
| 10 nM | 1.1 | 1.9 | 2.3 | 0.4 |
| Average | 0.9 | 1.6 | 2.0 | 0.4 |
| STD | 0.2 | 0.3 | 0.3 | 0.04 |

TABLE 18
off rates (koff) in Biacore for binding of different
concentrations (0.3-10 nM) of antibodies to MuSK
at neutral versus endosomal pH (pH 5.5).

| koff (10-4/s) | 3D10a | 4D3 | 3F6c | 3D9b |
|---|---|---|---|---|
| pH 7.4 | 1.6 | 1.4 | 0.8 | 1.2 |
| pH 5.5 | 0.9 | 1.6 | 2.0 | 0.4 |
| Fold difference (5.5/7.4) | 0.6 | 1.1 | 2.5 | 0.3 |

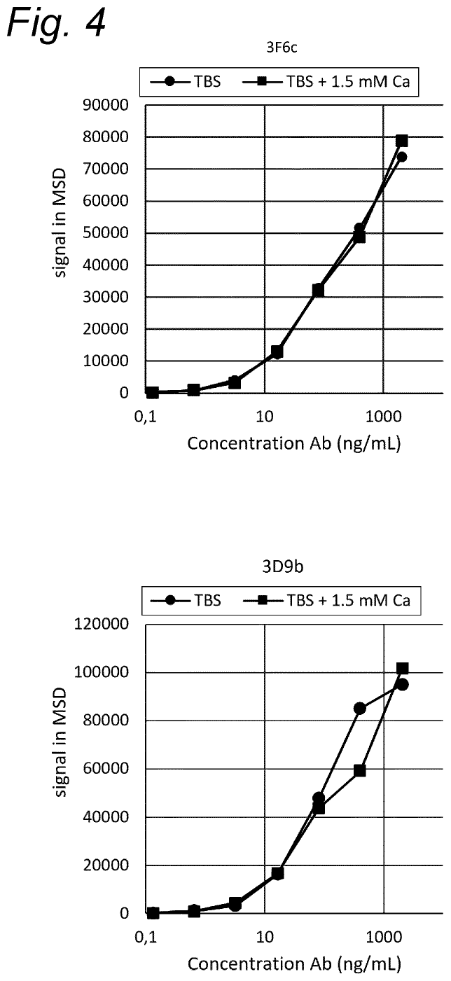

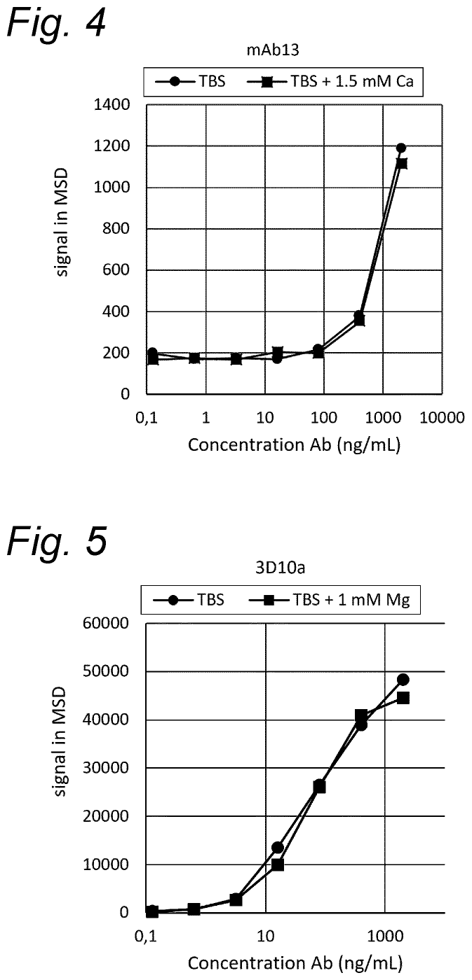

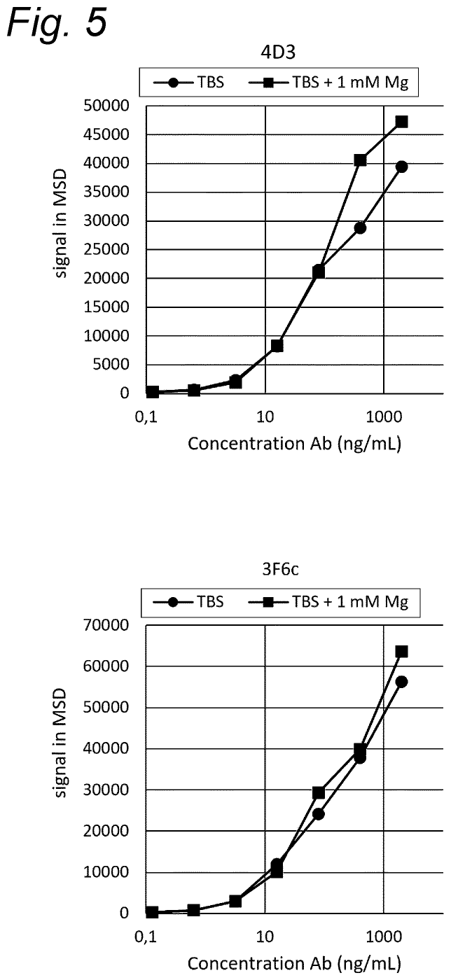

### Example 8: Ca and Mg Dependent Binding to MuSK Using Mesoscale

Whereas a pH-dependent antibody utilizes the pH difference—that is, the difference in proton concentration—between plasma and endosome, Calcium ion concentration is another factor reported to be different between plasma and endosome. Calcium ion concentration in plasma is 1.2-2 mM and that in the endosome is 3-30 μM. Since the difference in Calcium ion concentration is even greater than the difference in proton concentration, Calcium-dependent antibody may achieve more strongly dependent antigen binding than a pH-dependent antibody (mAntibodies 8:1, 65-73; January 2016). Therefore, we tested whether the affinity of our antibodies is influenced by the presence of calcium. The results demonstrate that there is no effect of Ca on the affinity of the antibodies for MuSK under the current conditions (FIG. 4).

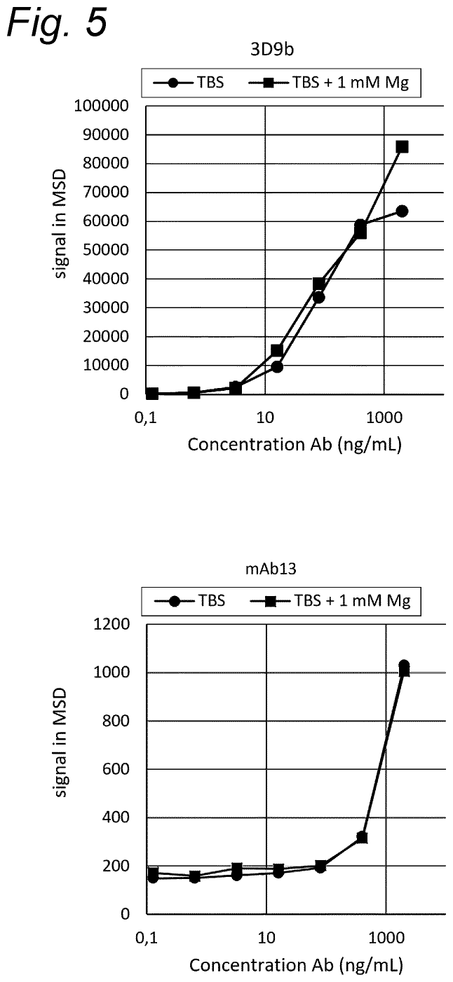

Next, we tested the role of magnesium since blood contains high concentrations of Magnesium. Also intracellular there is magnesium present, since this is essential for ATP to be biologically active (amongst other functions). Some numbers: in mammalian cell 10 mM (bound), 0.5 mM (free) and in blood plasma 1 mM. We compared the affinity of our antibodies in the absence and presence of 1 mM Mg in order to mimic the affinity of the antibodies for MuSK in the blood stream. Affinity was determined using Mesoscale. The results demonstrate that there is no effect of Mg on the affinity of the antibodies for MuSK (except for 4D3 maybe (to be confirmed)) under the current conditions (FIG. 5).

### Example 9: Epitope Mapping in ELISA: Binding to MuSK Domains and Epitope Binning

MuSK comprises three immunoglobulin (Ig)-like domains, a cysteine-rich domain (CRD) related to that of the Wnt-receptor Frizzled, a transmembrane helix, and a cytoplasmic tyrosine kinase domain (FIG. 11). The first and second Ig-like domains of MuSK (Ig1-2) are crucial for agrin-induced MuSK activation. Ig1-2 are configured in a linear, semi-rigid arrangement, and hydrophobic residues on the surface of Ig1 are required for activation of MuSK by agrin. The Fz-CRD is dispensable for agrin-induced MuSK activation and AChR clustering, but its absence affects the ability of MuSK to co-cluster with rapsyn and AChRs. Therefore, the MuSK Fz-CRD might mediate a direct interaction between MuSK and AChR on the cell surface or could bind to another transmembrane protein to bridge MuSK and cytoplasmic rapsyn. (J Mol Biol. 2009, 16; 393(1): 1-9)

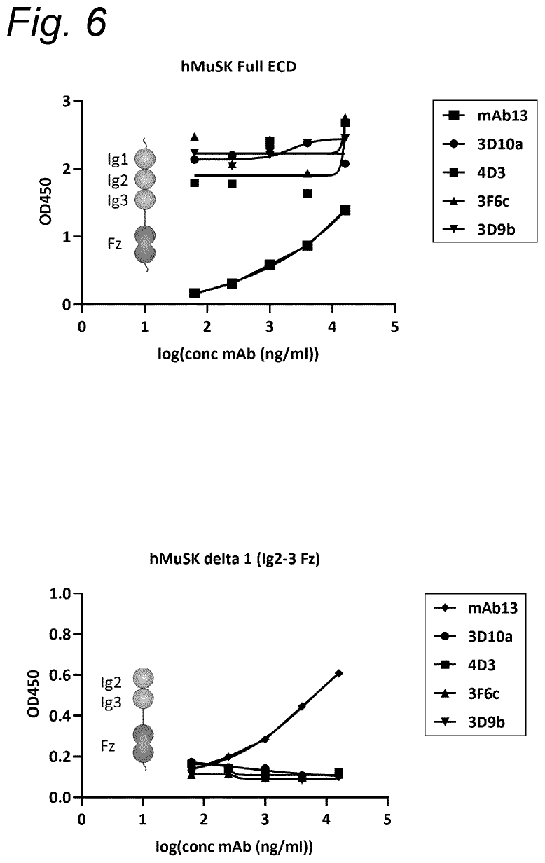

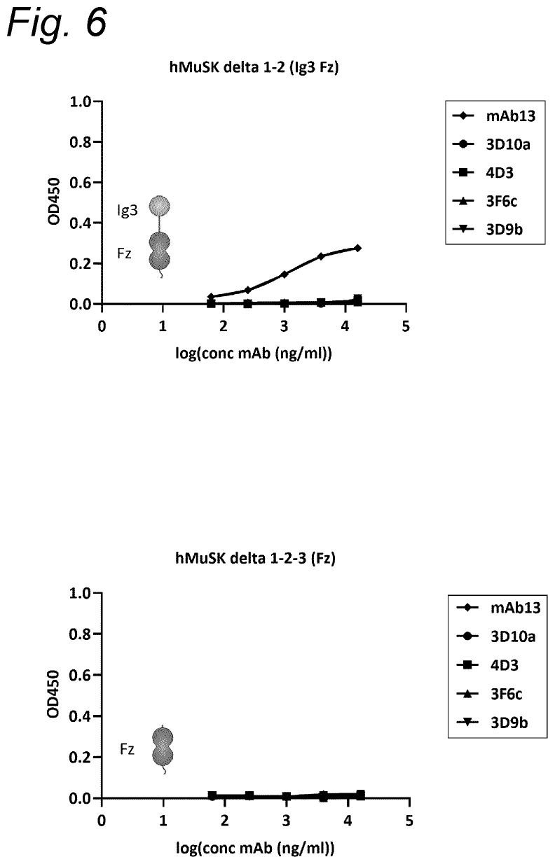

For the MuSK domain binding ELISA, binding of anti-MuSK antibodies to FLAG captured human monomeric MuSK and truncated variants was tested (FIG. 6). 3D9b, 3F6c, 3D10a, 4D3 and 3B5 bound only to full length MuSK, no binding was observed against the truncated MuSK variants. Therefore, we conclude that our antibodies bind to the Ig1 domain.

For the epitope binning ELISA, a plate was coated with anti-MuSK Fab and human monomeric MuSK was added after blocking the plate. Next the different antibodies were added and allowed to bind. Detection of the anti-MuSK binding was done using goat anti-human Fc HRP that does not recognize the CH1 domain of the coated Fab. The results demonstrate the following (Table 19):

3B5 binds to a different epitope than the other MuSK antibodies described here. They outcompete each other for the same epitope except for 3B5. This antibody induces MG when injected in wild type naive NOD-SCID mice and therefore is not suited for further development in the clinic (data not shown).

The other antibodies and their variants share an overlapping epitope in this assay.

TABLE 19
OD 450 nm in the epitope binning ELISA

| | 3B5wt | 3D10a | 4D3 | 3F6c | 3D9b |
|---|---|---|---|---|---|
| 3B5 | 0.048 | 1.875 | 1.955 | 2.029 | 1.992 |
| 3D10a | 2.119 | 0.049 | 0.062 | 0.162 | 0.115 |
| 4D3 | 0.882 | 0.008 | 0.023 | 0.100 | 0.061 |
| 3F6c | 2.196 | 0.057 | 0.076 | 0.156 | 0.133 |
| 3D9b | 2.662 | 0.060 | 0.086 | 0.186 | 0.150 |

### Example 10: Epitope Mapping in Biacore

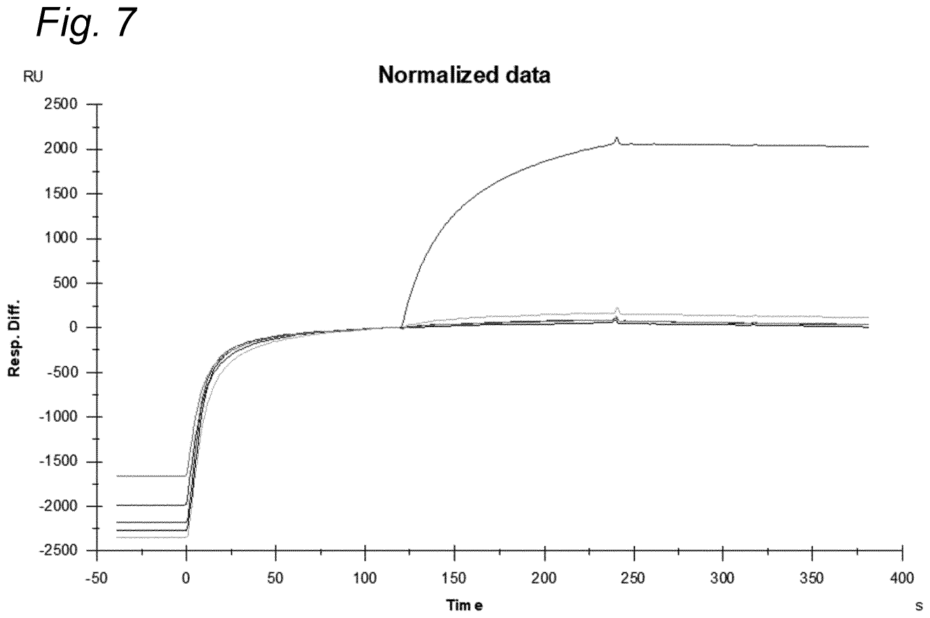

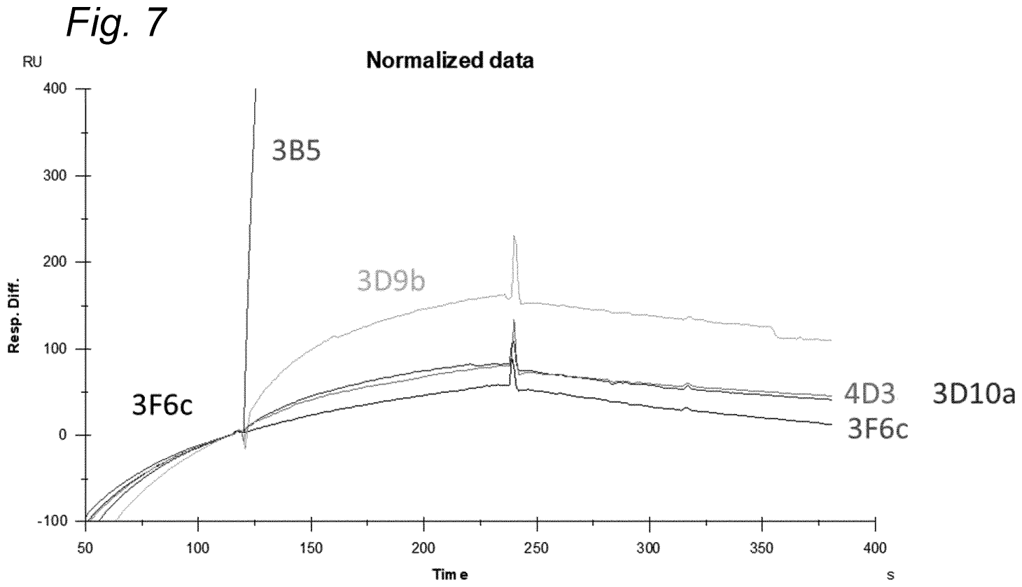

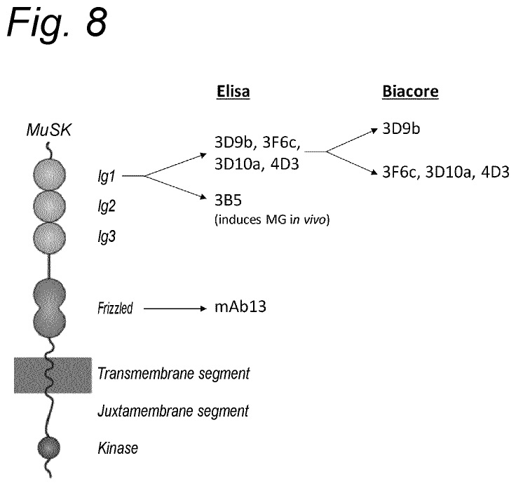

The aim of this experiment was to assess, using SPR technology, if the 4 pre-lead antibodies and 3B5 recognize a similar or different epitope(s) for binding to human MuSK (Tables 21-24). As 3D10a, 4D3 and 3F6c show similar behavior in this experiment and cannot bind together, it can be concluded that they recognize the same epitope on MuSK. However, 3D9b was still able to bind on MuSK after saturation with 3D10a, 4D3 or 3F6c. Nevertheless, the binding amplitude of 3D9b was diminished with similar intensity independent if MuSK was pre-saturated with 3D10a, 4D3 or 3F6c (FIG. 7). This reduced amplitude can be explained by the fact that the binding of 3D10a, 4D3 and 3F6c creates a conformational change in MuSK, thereby reducing the availability of the epitope of 3D9b. When MuSK is saturated with 3D9b similar behavior is again observed for 3D10a, 4D3 and 3F6c. They do not bind anymore or even induce displacement of 3D9b. 3B5 clearly recognizes a non-overlapping epitope then 3D10a, 4D3 and 3F6c. Pre-leads 3F6c and 3D10a probably stimulate the accessibility of the 3B5 epitope as the binding signal of 3B5 is at least 3 times higher than the amplitude of 3B5 on non-saturated MuSK.

In conclusion, this experiment suggests that 3D10a, 4D3 and 3F6c recognize a similar/identical epitope on MuSK. For 3D9b it's assumed that it recognizes a partial overlapping epitope compared with 3D10a, 4D3 and 3F6c or that the binding of 3D9b is diminished by a conformational change in MuSK caused by 3D10a, 4D3 and 3F6c. Lastly, 3B5 binds to a non-overlapping epitope on MuSK compared to 3D10a, 4D3, 3F6c and 3D9b.

TABLE 20
signal obtained in Biacore (SPR) for binding of different antibodies
to a MuSK coated chip with and without saturation first with 3D10a

| | | amplitude | amplitude | | |
|---|---|---|---|---|---|
| | | of prelead | of prelead | | |
| | | without | after | | normalized |
| | | presaturating | presaturating | extra | extra |
| | | MuSK with | MuSK with | amplitude | amplitude |
| 3D10a | Prelead | 3D10a (RU) | 3D10a (RU) | (%) | (%) |
| 500 nM | 4D3 | 2617 | 42 | 1.6 | 0.0 |
| 500 nM | 3F6c | 2509 | 26 | 1.0 | −0.6 |
| 500 nM | 3B5 | 658 | 2262 | 344 | 342.2 |
| 500 nM | 3D9b | 2113 | 169 | 8.0 | 6.4 |
| 500 nM | 3D10a | 3128 | 56 | 1.8 | 0.2 |

TABLE 21
signal obtained in Biacore (SPR) for binding of different antibodies
to a MuSK coated chip with and without saturation first with 4D3

| | | amplitude | amplitude | | |
|---|---|---|---|---|---|
| | | of prelead | of prelead | | |
| | | without | after | | normalized |
| | | presaturating | presaturating | extra | extra |
| | | MuSK with | MuSK with | amplitude | amplitude |
| 4D3 | Prelead | 4D3 (RU) | 4D3 (RU) | (%) | (%) |
| 500 nM | 4D3 | 859 | 45 | 5.2 | 0.0 |
| 500 nM | 3F6c | 1013 | 59 | 5.8 | 0.6 |
| 500 nM | 3B5 | 151 | 141 | 93 | 88.1 |
| 500 nM | 3D9b | 1097 | 163 | 14.9 | 9.6 |
| 500 nM | 3D10a | 951 | 68 | 7.2 | 1.9 |

TABLE 22
signal obtained in Biacore (SPR) for binding of different antibodies
to a MuSK coated chip with and without saturation first with 3F6c

| | | amplitude | amplitude | | |
|---|---|---|---|---|---|
| | | of prelead | of prelead | | |
| | | without | after | | normalized |
| | | presaturating | presaturating | extra | extra |
| | | MuSK with | MuSK with | amplitude | amplitude |
| 3F6c | Prelead | 3F6 (RU) | 3F6 (RU) | (%) | (%) |
| 500 nM | 4D3 | 2390 | 68 | 2.8 | 1.1 |
| 500 nM | 3F6c | 3638 | 64 | 1.8 | 0.0 |
| 500 nM | 3B5 | 562 | 2230 | 397 | 395.0 |
| 500 nM | 3D9b | 1663 | 191 | 11.5 | 9.7 |
| 500 nM | 3D10a | 2367 | 75 | 3.2 | 1.4 |

TABLE 23
signal obtained in Biacore (SPR) for binding of different antibodies
to a MuSK coated chip with and without saturation first with 3D9b

| | | amplitude | amplitude | | |
|---|---|---|---|---|---|
| | | of prelead | of prelead | | |
| | | without | after | | normalized |
| | | presaturating | presaturating | extra | extra |
| | | MuSK with | MuSK with | amplitude | amplitude |
| 3D9b | Prelead | 3D9b (RU) | 3D9b (RU) | (%) | (%) |
| 500 nM | 4D3 | 188.4 | −3 | −1.6 | −2.6 |
| 500 nM | 3F6c | 235 | −5 | −2.1 | −3.1 |
| 500 nM | 3B5 | 399 | 2149 | 539 | 537.6 |
| 500 nM | 3D9b | 3970 | 40 | 1.0 | 0.0 |
| 500 nM | 3D10a | 189 | −1 | −0.5 | −1.5 |

### Example 11: MuSK Phosphorylation Assay

In order to evaluate the extent of MuSK phosphorylation induced by our pre-lead agonistic MuSK antibodies, an in vitro MuSK phosphorylation assay using mouse C2C12 myotubes was used. 3D10a, 4D3 and 3F6c have similar MuSK phosphorylation levels as agrin. Moreover, 3D9b can induce almost double the amount of MuSK phosphorylation compared agrin. These data support capacity of our pre-lead Antibodies to induce MuSK phosphorylation be MuSK agonists, to similar levels or more than agrin (Table 24).

TABLE 24
Table presenting MuSK phosphorylation amounts compared to
agrin of the pre-lead agonistic MuSK Antibodies and mAb13.

| | | | | | | Neg |
|---|---|---|---|---|---|---|
| Phosphorylation | 3D10a | 4D3 | 3F6c | 3D9b | mAb13 | Ctrl* |
| % versus | 83 ± 3 | 112 ± 3 | 100 ± 12 | 205 ± 29 | 61 ± 6 | 11 ± 5 |
| agrin | | | | | | (N = 4) |

(All numbers are averages normalized against an agrin control and N = 3 unless otherwise specified).
*The negative control is anti-biotin. (produced by LUMC)

### Example 12: AChR Clustering Assay

Transmission at the NMJ depends on proper clustering of AChRs on the muscle membrane exactly opposing the ACh releasing presynaptic nerve terminal. This clustering depends on agrin-LRP4-MuSK signalling. Agrin is released from the motor neuron presynapse and activates LRP4 binding to MuSK, inducing MuSK dimerization and transautophosphorylation. MuSK phosphorylation will consequentially lead to AChR clustering through (mostly unknown) intracellular signalling. As can be seen in Table 26-27, all the agonistic anti-MuSK pre-lead Antibodies can induce AChR clustering to a similar level as the benchmark mAb #13 Ab (independent from agrin). Interestingly, mAb #13 seems to be slightly better at inducing smaller (number of ≥3 μm² clusters) AChR clusters in vitro. However, our pre-lead anti-MuSK Antibodies seem to be better at inducing large AChR clusters (number of 15 pmt clusters) in vitro. What the importance is of small and large AChR clusters in vivo is unclear, therefore injecting the anti-MuSK Antibodies in vivo will be essential to select a lead Ab.

TABLE 25
AChR clusters larger or equal to 15 μm (large clusters) compared
to agrin upon incubation with 1.1 μg/ml pre-lead agonistic MuSK
Ab. (All numbers are averages normalized against 100% agrin as control
and N = 3 unless otherwise specified) The N = 8 negative
control is anti-biotin, while the N = 3 negative control is Motavizumab.

| | | | | | | | Neg |
|---|---|---|---|---|---|---|---|
| Exp | Batch | 3D10a | 4D3 | 3F6c | 3D9b | mAb13 | Ctrl |
| 1 | Argenx | 52 ± 1 | 84 ± 11 | 56 ± 3 | 80 ± 12 | 52 ± 4 | 12 ± 2 |
| | | | | | | | (N = 8) |
| 2 | Argenx | 73 ± 9 | 58 ± 5 | 70 ± 6 | 74 ± 13 | 60 ± 6 | — |
| | | | (N=5) | | (N = 6) | | |
| 3 | UPX | 61 ± 4 | 59 ± 4 | 57 ± 5 | 70 ± 4 | 68 ± 11 | 22 ± 3 |
| | | (N = 6) | (N = 6) | (N = 6) | (N = 6) | | |

TABLE 26
AChR clusters larger or equal to 3 μm compared to agrin upon incubation with
1.1 μg/ml pre-lead agonistic MuSK Ab. (All numbers are averages normalized
against 100% agrin as control and N = 3 unless otherwise specified) The
N = 8 negative control is anti-biotin, while the N = 3 negative control is Motavizumab.

| | | | | | | | Neg |
|---|---|---|---|---|---|---|---|
| Exp | Batch | 3D10a | 4D3 | 3F6c | 3D9b | mAb13 | Ctrl |
| 1 | Argenx | 64 ± 2 | 102 ± 12 | 71 ± 6 | 89 ± 17 | 84 ± 5 | 21 ± 3 |
| | | | | | | | (N = 8) |
| 2 | Argenx | 99 ± 9 | 73 ± 7 | 105 ± 7 | 95 ± 15 | 98 ± 3 | |
| | | | (N = 5) | | (N = 6) | | |
| 3 | UPX | 71 ± 4 | 72 ± 4 | 69 ± 7 | 91 ± 5 | 115 ± 16 | 35 ± 5 |
| | | (N = 6) | (N = 6) | (N = 6) | (N = 6) | | |

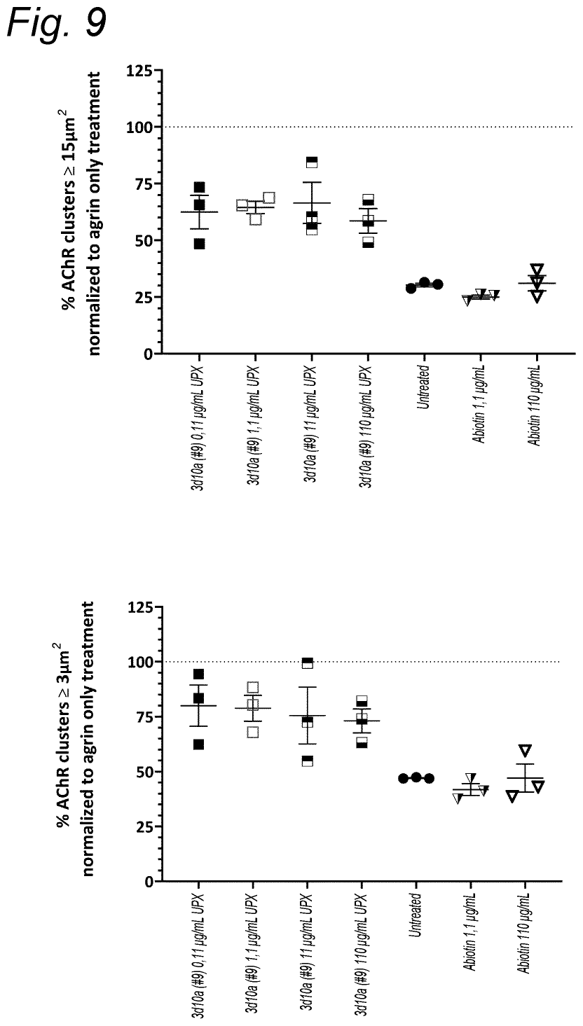

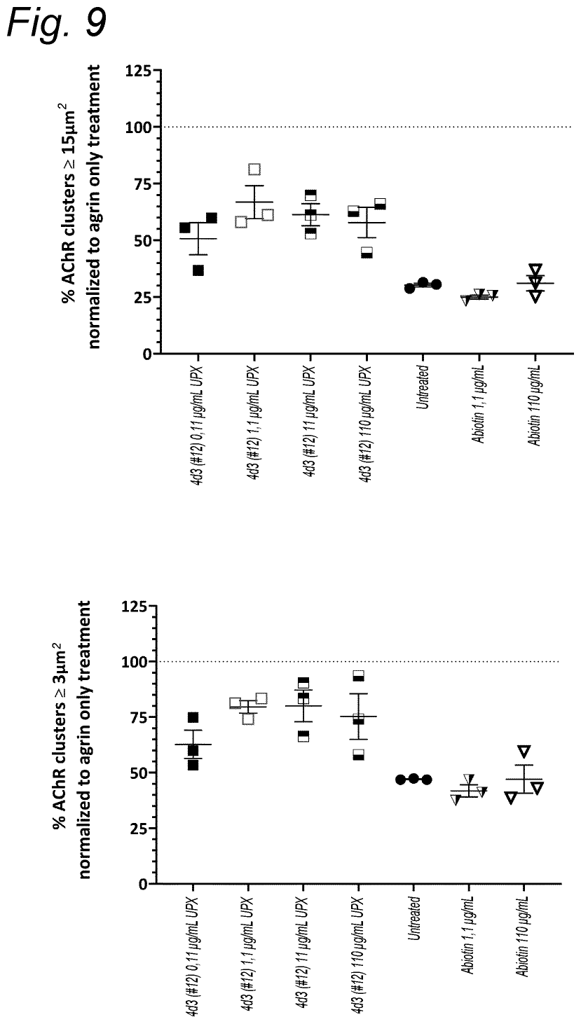

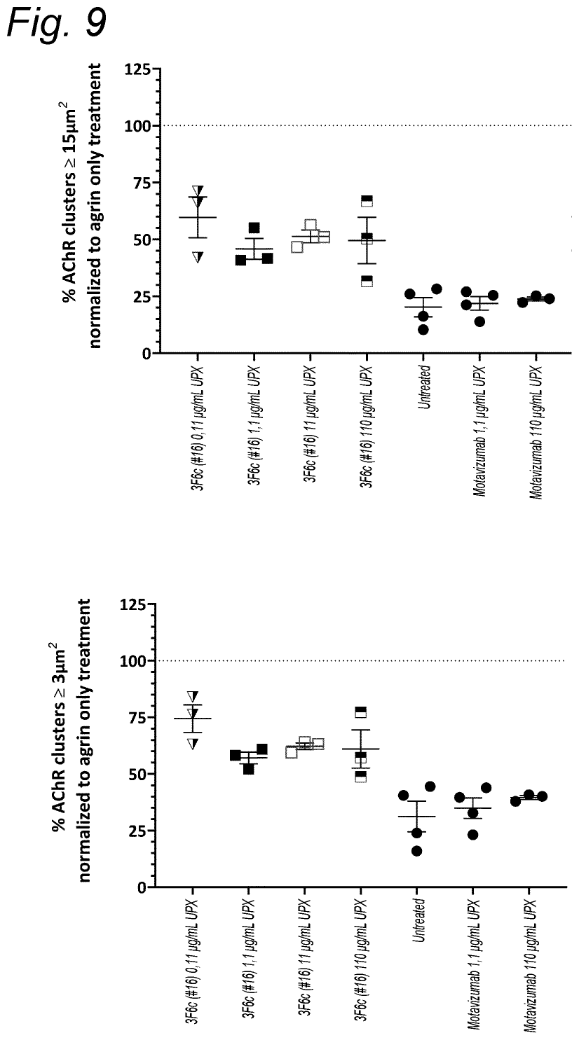

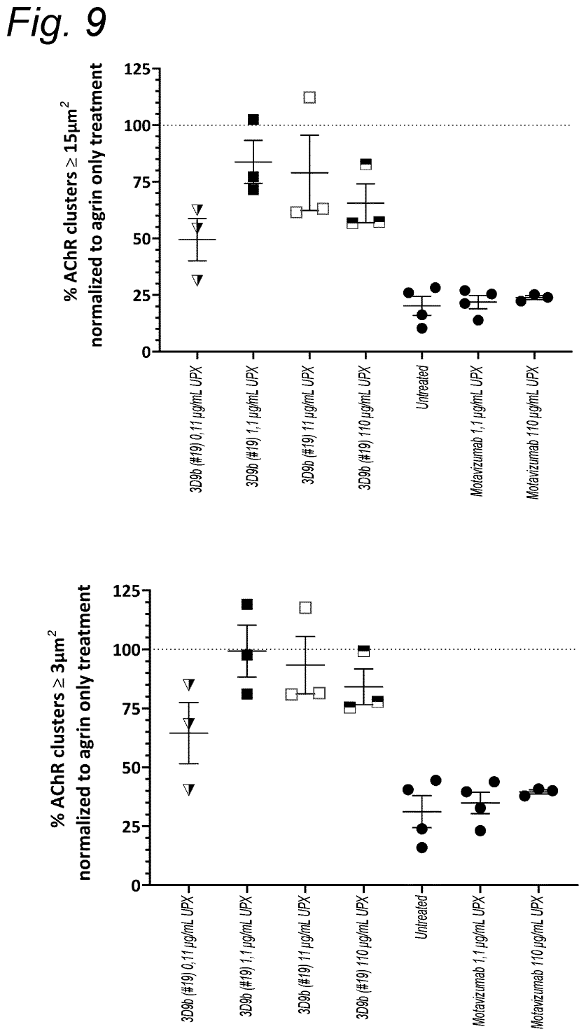

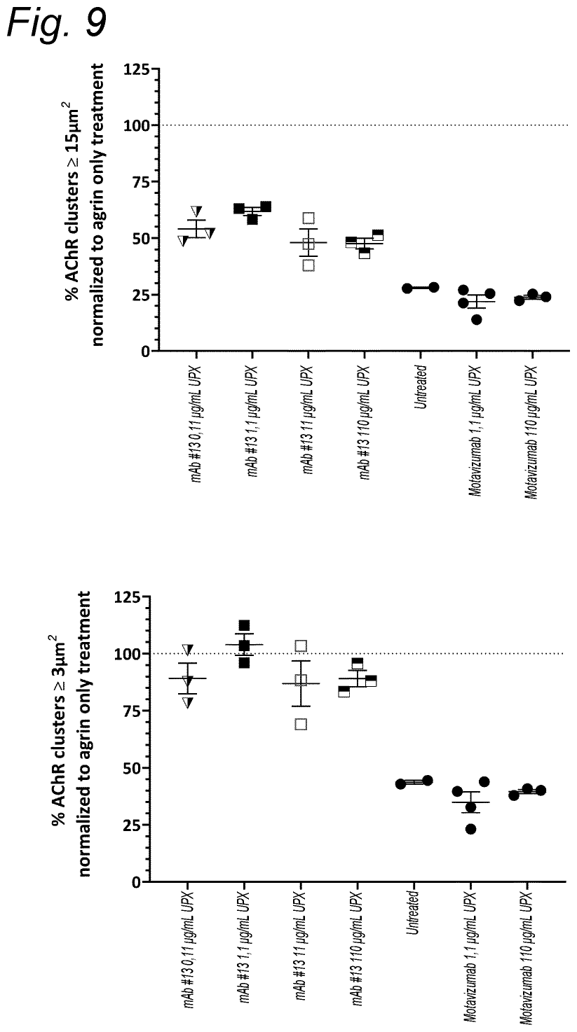

We did not observe a significant increase in AChR clusters when adding higher concentrations of agonistic MuSK Ab.”. This data is important since it suggests that overdosing will have no effect on AChR cluster formation, at least not in vitro (Table 27-28 and FIG. 9).

TABLE 27
AChR clusters larger or equal to 15 μm (large clusters) compared to agrin
upon incubation with 1.1 μg/ml pre-lead agonistic MuSK Ab. (All numbers are
averages normalized against an agrin control and N = 3 unless otherwise specified)

| Concentration (μg/ml) | 3D10a | 4D3 | 3F6c | 3D9b | mAb13 | Motavizumab |
|---|---|---|---|---|---|---|
| 0.11 | 62 ± 6 | 44 ± 5 | 60 ± 7 | 49 ± 8 | 54 ± 3 | |
| 1.1 | 64 ± 2 | 58 ± 1 | 46 ± 4 | 84 ± 8 | 62 ± 1 | 18 ± 2 |
| | | | | | | (N = 4) |
| 11 | 61 ± 11 | 61 ± 4 | 51 ± 2 | 79 ± 14 | 48 ± 5 | |
| 110 | 58 ± 4 | 58 ± 5 | 50 ± 8 | 66 ± 7 | 48 ± 2 | 24 ± 1 |

TABLE 28
AChR clusters larger or equal to 3 μm compared to agrin upon incubation with
a dose-concentration range of pre-lead agonistic MuSK Ab. (All numbers are averages
normalized against an agrin control and N = 3 unless otherwise specified)

| Conc. (μg/mL) | 3D10a | 4D3 | 3F6c | 3D9b | mAb13 | Motavizumab |
|---|---|---|---|---|---|---|
| 0.11 | 80 ± 8 | 63 ± 5 | 74 ± 5 | 65 ± 11 | 89 ± 5 | |
| 1.1 | 79 ± 5 | 80 ± 2 | 57 ± 2 | 99 ± 9 | 104 ± 4 | 39 ± 5 |
| | | | | | | (N = 4) |
| 11 | 76 ± 11 | 80 ± 6 | 62 ± 1 | 93 ± 10 | 87 ± 8 | |
| 110 | 73 ± 4 | 75 ± 8 | 61 ± 7 | 84 ± 6 | 89 ± 3 | 40 ± 1 |

### Example 13: AChR Clustering Assay in the Presence of MG Patient Derived Polyclonal Antibodies

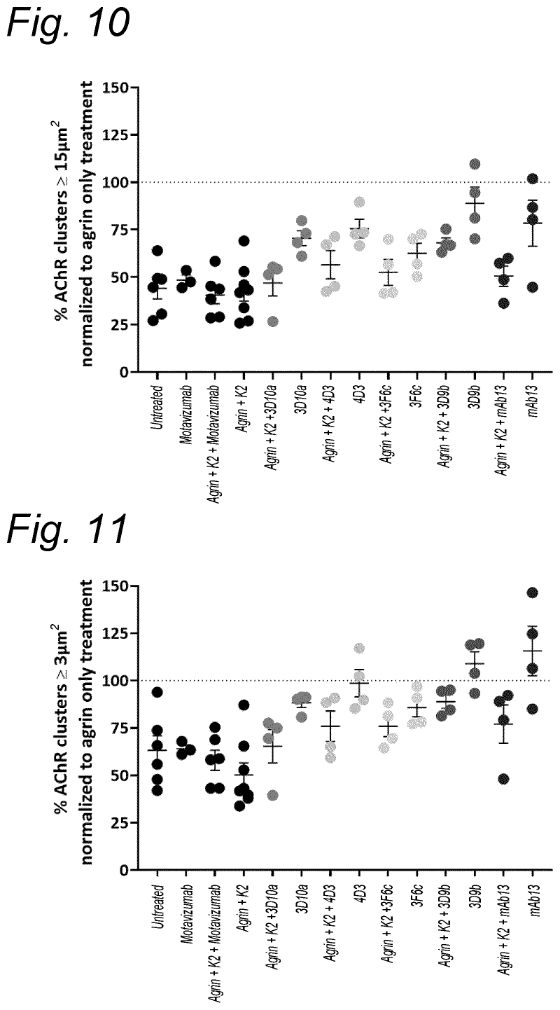

Agonistic anti-MuSK Antibodies could potentially be used as a therapeutic for MuSK induced MG. Therefore, it is important to see if these anti-MuSK Ab can outcompete or counteract the inhibitory effects of polyclonal autoantibodies derived from MuSK MG patients (K2). This will also reveal the binding capacity to MuSK in presence of other MuSK Antibodies, which can aid the lead-selection process. Interestingly, in this in vitro test 4D3, 3D9b and 3F6c perform the best as the therapeutic setting shows AChR cluster numbers close to the antibody-only treatment setting and the relative levels of AChR clustering are the highest. (FIGS. 10 and 11)

### Example 14: AChR Staining of Diaphragm Neuromuscular Junctions with AF488-BTx

### Methods

### Fluorescence Microscopy of Neuromuscular Junctions

We also analyzed morphology of NMJs in diaphragm muscles of the MuSK antibody treated mice. Diaphragm strips were fixed in 1% paraformaldehyde in PBS, washed in PBS and incubated for 3.5 h with 1 μg/ml Alexa Fluor 488 conjugated alpha-bungarotoxin, followed by PBS wash (for 48 hrs), all at room temperature. AChR receptor staining at diaphragm NMJ was visualized. Muscles were whole-mounted and randomly chosen NMJ viewed under an epi-fluorescent microscopy using identical settings.

Antibodies

Antibodies (3D10a, 4D3, 3D9b and 3F6c) were produced by U-protein Express (UPX) using the hIgG1-LALA-delK backbone. Control antibody 3B5 was produced by Evitria, initiated by LUMC and has an hIgG4 S228P backbone. The references for the reports and relevant information about the antibodies is summarized in Table 30. All antibodies from UPX were formulated in PBS-0.02% Tween80. The 3B5 antibody was stored in PBS. Endotoxin levels were determined at Eurofins for the UPX antibodies only.

TABLE 29
information about the antibodies used in this report

| | 3D10a | 4D3 | 3F6c | 3D9b | 3B5 |
|---|---|---|---|---|---|
| UPX report reference | 2019-230 | | | | Evitria by LUMC |
| Batch nr | 3832 | 3833 | 3834 | 3835 | 1 (No. 12022) |
| Human identity (%) | 99.4 | 100 | 100 | 100 | 87.5 |
| Production yield (mg/L) HEK293 | 204 | 156 | 224 | 263 | 193 (produced in |
| | | | | | CHO cells) |
| Concentration (mg/mL) | 7.3 | 7.3 | 7.1 | 6.3 | 1.1 |
| Aggregates % aggregates after | <1% | <1% | <1% | <1% | Not determined |
| mAbselect + gelfiltration | | | | | |
| Endotoxin (EU/mg) | 0.0068 | 0.069 | 0.07 | 0.008 | Not determined |

### NOD/SCID Mouse Study—Multiple Dosing of Anti-MuSK

To circumvent the potential problem of a mouse immune response against repetitively injected human IgG we used immunodeficient NOD.CB17-Prkdcscid/J mice (Shultz et al., 1995). Original breeders were purchased from Jackson Laboratory (Bar Harbor). Mice were bred and housed in sterile individually ventilated cages in the Leiden University Medical Centre animal facilities. Sterilized food and drinking water was provided ad libitum. We used 5 female and 5 male NOD/SCID mice, aged 5 weeks at the start, for dosing with two times 20 mg/kg per week. Each pre-lead anti-MuSK hIgG1 antibody or positive control (3B5 hIgG4 S228P): inducing MG-like phenotypes) was tested in one male and one female mouse. Mice were injected twice a week (on Mondays and Thursdays) intraperitoneally with a dose of 20 mg/kg (based on the body weights on the third day prior to the first injection). Anti-MuSK hIgG1 antibodies were diluted in a volume of 250 μl sterile PBS. Positive control injection volume was in ˜400 ul sterile PBS (because the stock solution was not concentrated enough to limit the injection volume to 250 μl). Daily, the body weight of each mouse was determined and neuromuscular performance was assessed as described below. At the endpoint day (see Table 30), electromyographical testing under anesthesia was performed, followed by CO₂ euthanasia and dissection of diaphragm muscle nerve preparations for ex vivo muscle contraction tests and histology (see below). For rough comparisons, two untreated NOD/SCID mice (one male/one female) were included in the endpoint analyses. All experiments were carried out according to Dutch law and Leiden University guidelines. The study was done blinded, except for 3B5 since a higher volume was injected and no blinding could be done.

TABLE 30
Dosing scheme details

| antibody | 3D10a | 4D3 | 3F6c | 3D9b | 3B5 |
|---|---|---|---|---|---|
| #injections | 8 | 7 | 7 | 7 | 6 |
| Treatment duration (days) | 25 | 24 | 23 | 22 | 21 |
| Time between last injection | 1 | 3 | 2 | 1 | 4 |
| and endpoint analyses (days) | | | | | |

### Results

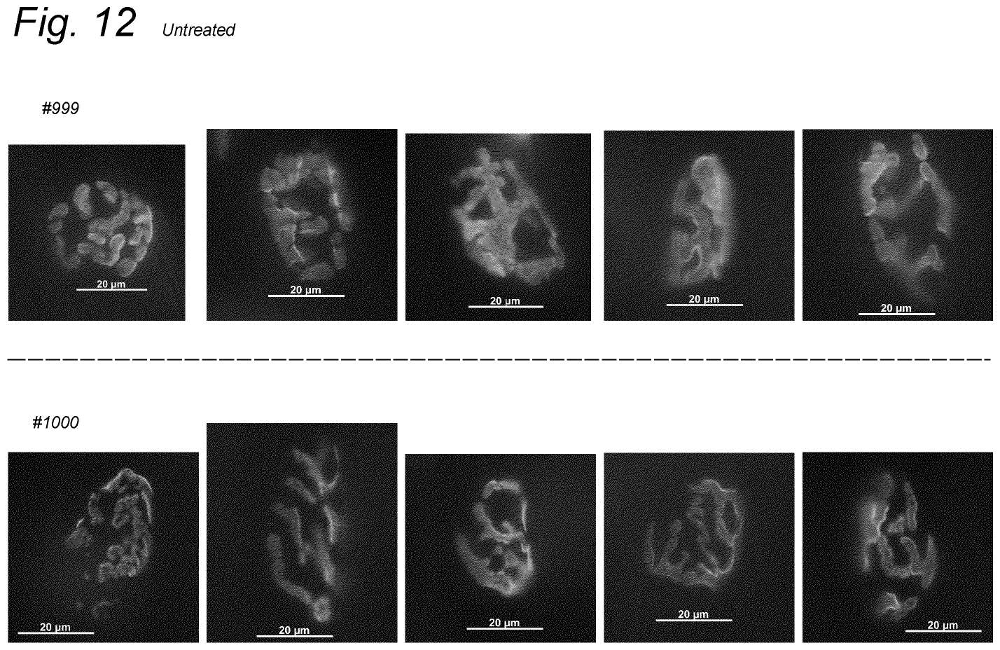

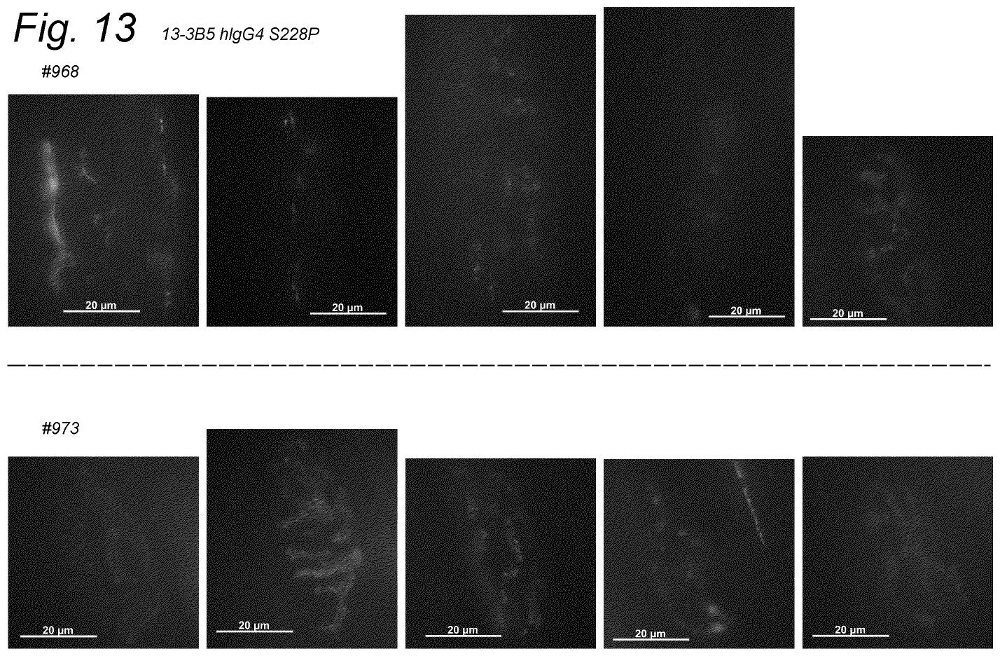

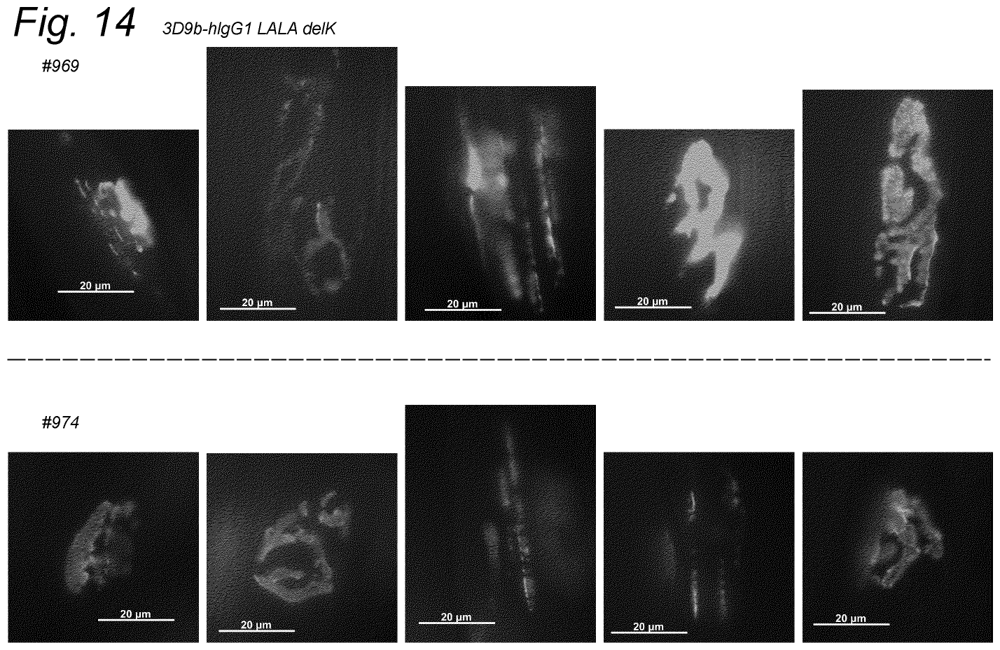

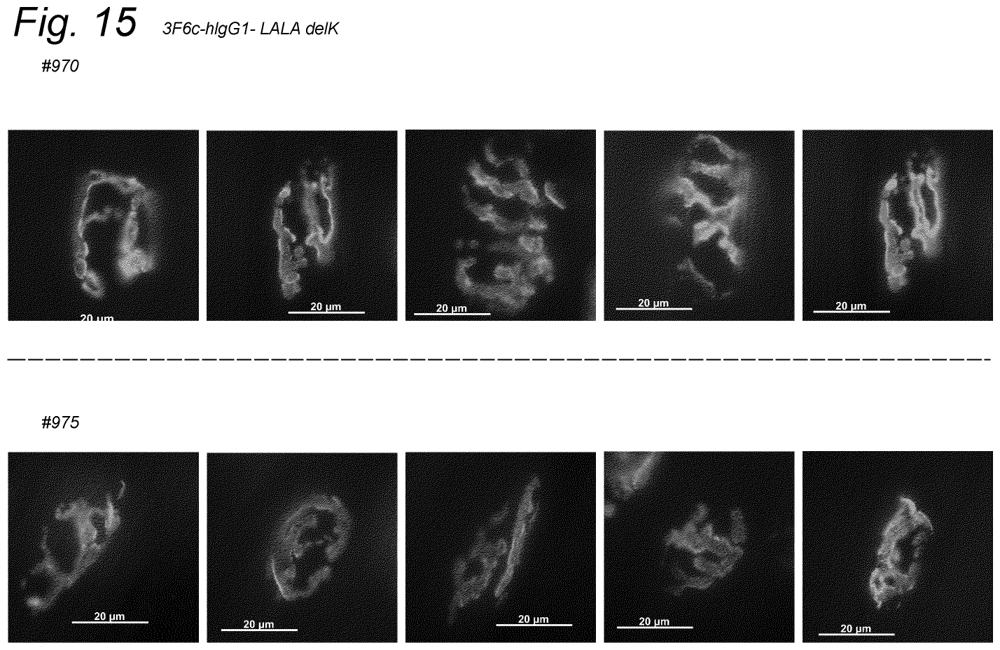

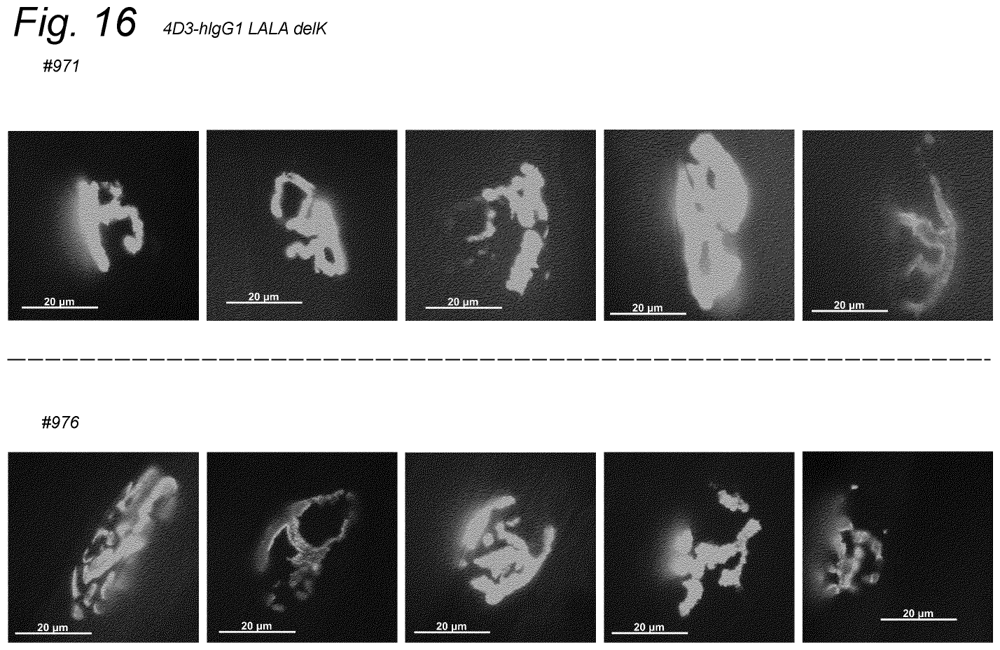

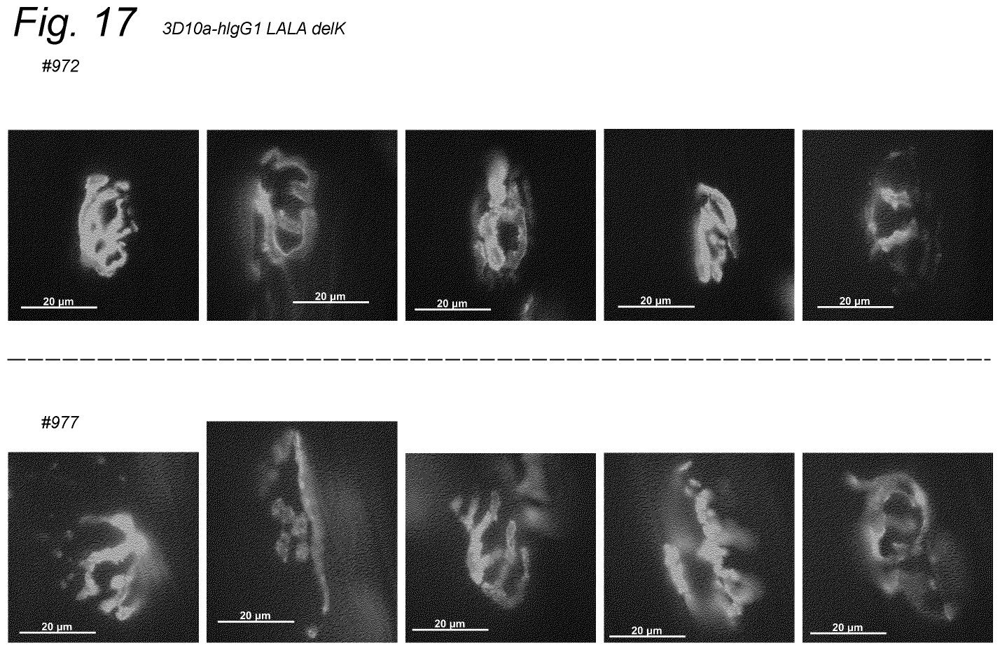

Diaphragms from all mice were stained for AChRs to enable NMJ imaging. Epi-fluorescent microscopy of NMJ from untreated mice showed the expected “pretzel-like” morphological (FIG. 12). The samples of untreated mice where used to optimize the imaging settings, so that these can be used (and not changed anymore) when imaging the NMJs of the treated mice. NMJs of mice treated with 3B5 showed severe morphological abnormalities (FIG. 13), including very small and less intensely stained AChR clusters (not quantified). Many NMJs had an irregular, dispersed and punctuate staining pattern. A proportion of NMJs showed remarkable striping, with multiple elongated clusters running in parallel along the longitudinal muscle fiber axis. Mice treated with 3D9b showed a mixed set of NMJs, a minority had severe morphological abnormalities similar to those seen in the 3B5, others appeared as the untreated condition (FIG. 14). NMJs of mice treated with 3F6c showed similar morphology and AChR intensity as the untreated NMJs (FIG. 15). Interestingly, NMJs of 4D3 or 3D10a treated mice showed similar morphology as untreated mice, but with an increased AChR signal intensity. This possibly suggest more or more densely packed AChRs (FIG. 16-17).

1-24. (canceled)

25. An antibody that specifically binds to MuSK, wherein the antibody comprises:
(i) a heavy chain variable region (VH) comprising the HCDR1, HCDR2, and HCDR3 amino acid sequences of the VH amino acid sequence of SEQ ID NO: 37; and a light chain variable region (VL) comprising the LCDR1, LCDR2, and LCDR3 amino acid sequences of the VL amino acid sequence of SEQ ID NO: 57;
(ii) a VH comprising the HCDR1, HCDR2, and HCDR3 amino acid sequences of the VH amino acid sequence of SEQ ID NO: 36; and a VL comprising the LCDR1, LCDR2, and LCDR3 amino acid sequences of the VL amino acid sequence of SEQ ID NO: 56; or
(iii) a VH comprising the HCDR1, HCDR2, and HCDR3 amino acid sequences of the VH amino acid sequence of SEQ ID NO: 38; and a VL comprising the LCDR1, LCDR2, and LCDR3 amino acid sequences of the VL amino acid sequence of SEQ ID NO: 58.

26. The antibody of claim 25, wherein the antibody comprises
(i) the HCDR1, HCDR2, and HCDR3 amino acid sequences of SEQ ID NOs: 42, 47, and 52, respectively; and LCDR1, LCDR2, and LCDR3 amino acid sequences of SEQ ID NOs: 62, 67, and 72, respectively;
(ii) the HCDR1, HCDR2, and HCDR3 amino acid sequences of SEQ ID NOs: 41, 46, and 51, respectively; and LCDR1, LCDR2, and LCDR3 amino acid sequences of SEQ ID NOs: 61, 66, and 71, respectively; or
(iii) the HCDR1, HCDR2, and HCDR3 amino acid sequences of SEQ ID NOs: 43, 48, and 53, respectively; and LCDR1, LCDR2, and LCDR3 amino acid sequences of SEQ ID NOs: 63, 68, and 73, respectively.

27. The antibody of claim 25, wherein:
(i) the VH comprises the amino acid sequence of SEQ ID NO:37 or an amino acid sequence at least 90%, 95%, 97%, 98% or 99% identical thereto and the VL comprises the amino acid sequence of SEQ ID NO:57 or an amino acid sequence at least 90%, 95%, 97%, 98% or 99% identical thereto (3F6c);
(ii) the VH comprises the amino acid sequence of SEQ ID NO:36 or an amino acid sequence at least 90%, 95%, 97%, 98% or 99% identical thereto and the VL comprises the amino acid sequence of SEQ ID NO:56 or an amino acid sequence at least 90%, 95%, 97%, 98% or 99% identical thereto (3D10a); or
(iii) the VH comprises the amino acid sequence of SEQ ID NO:38 or an amino acid sequence at least 90%, 95%, 97%, 98% or 99% identical thereto and the VL comprises the amino acid sequence of SEQ ID NO:58 or an amino acid sequence at least 90%, 95%, 97%, 98% or 99% identical thereto (4D3).

28. The antibody of claim 25, wherein the antibody comprises:
(i) a heavy chain comprising the amino acid sequence of SEQ ID NO:131 or an amino acid sequence at least 90%, 95%, 97%, 98% or 99% identical thereto and a light chain comprising the amino acid sequence of SEQ ID NO:132 or an amino acid sequence at least 90%, 95%, 97%, 98% or 99% identical thereto (3F6c);
(ii) a heavy chain comprising the amino acid sequence of SEQ ID NO:127 or an amino acid sequence at least 90%, 95%, 97%, 98% or 99% identical thereto and a light chain comprising the amino acid sequence of SEQ ID NO:128 or an amino acid sequence at least 90%, 95%, 97%, 98% or 99% identical thereto (3D10a); or
(iii) a heavy chain comprising the amino acid sequence of SEQ ID NO:129 or an amino acid sequence at least 90%, 95%, 97%, 98% or 99% identical thereto and a light chain comprising the amino acid sequence of SEQ ID NO:130 or an amino acid sequence at least 90%, 95%, 97%, 98% or 99% identical thereto (4D3).

29. The antibody of claim 25, wherein the antibody comprises a human heavy chain constant region.

30. The antibody of claim 29, wherein the human heavy chain constant region does not have a complement effector functionality.

31. The antibody of claim 29, wherein the human heavy chain constant region is a human IgG1, IgG2, IgG3, or IgG4 constant region.

32. The antibody of claim 29, wherein the human heavy chain constant region comprises one or more mutation.

33. The antibody of claim 32, wherein the human heavy chain constant region comprises L234A and L235A mutations, numbered according to the EU numbering system.

34. The antibody of claim 25, wherein the antibody comprises a human lambda light chain constant region or a human kappa light chain constant region.

35. The antibody of claim 25, wherein the antibody:
(i) agonizes MuSK; and/or
(ii) induces or increases induction of acetylcholine receptor (AChR) clustering at the neuromuscular junction (NMJ).

36. An isolated polynucleotide encoding a VH and/or VL of the antibody of claim 25.

37. The polynucleotide of claim 36, comprising a nucleotide sequence at least 90%, 95%, 97%, 98% or 99% identical to the nucleotide sequence set forth in SEQ ID NO: 27, 32, 26, 31, 28 or 33.

38. An expression vector comprising the isolated polynucleotide of claim 36.

39. A host cell or cell-free expression system comprising the isolated polynucleotide of claim 36.

40. A method of producing an antibody that specifically binds to MuSK, the method comprising culturing the host cell of claim 39 under conditions such that the polynucleotide is expressed and the antibody is produced.

41. A pharmaceutical composition comprising the antibody of claim 25 and a pharmaceutically acceptable carrier or excipient.

42. A method of treating a disease or condition related to an impaired neuromuscular transmission in a subject in need thereof, the method comprising administering to the subject a therapeutically effective amount of the antibody of claim 25.

43. The method of claim 42, wherein the disease or condition related to an impaired neuromuscular transmission is a neurodegenerative disease, optionally wherein the neurodegenerative disease is Amyotrophic Lateral Sclerosis (ALS), Alzheimer's disease, Huntington's disease, Parkinson's disease, frontotemporal dementia, Spinal Muscular Atrophy (SMA), congenital myasthenia, Emery-Dreifuss muscular dystrophy, Charcot-Marie tooth, Myasthenia Gravis, (post)poliomyelitis, aging or age-related muscle wasting, or sarcopenia.

44. A kit comprising the antibody of claim 25.


SEQUENCE LISTING (134 sequences)

SEQ ID NO: 1 - protein, 472 aa, Homo sapiens
LPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMT

SEQ ID NO: 2 - protein, 10 aa, Homo sapiens
EPKSCDKTHT

SEQ ID NO: 3 - protein, 5 aa, Homo sapiens
CPPCP

SEQ ID NO: 4 - protein, 8 aa, Homo sapiens
APELLGGP

SEQ ID NO: 5 - protein, 12 aa, Homo sapiens
ELKTPLGDTTHT

SEQ ID NO: 6 - protein, 50 aa, Homo sapiens
CPRCPEPKSCDTPPPCPRCPEPKSCDTPPPCPRCPEPKSCDTPPPCPRCP

SEQ ID NO: 7 - protein, 8 aa, Homo sapiens
APELLGGP

SEQ ID NO: 8 - protein, 7 aa, Homo sapiens
ESKYGPP

SEQ ID NO: 9 - protein, 5 aa, Homo sapiens
CPSCP

SEQ ID NO: 10 - protein, 8 aa, Homo sapiens
APEFLGGP

SEQ ID NO: 11 - skipped

SEQ ID NO: 12 - protein, 9 aa, Homo sapiens
CCVECPPCP

SEQ ID NO: 13 - protein, 7 aa, Homo sapiens
APPVAGP

SEQ ID NO: 14 - DNA, 1416 bp, synthetic construct
  misc_feature 1..1416: note = Human MuSK extracellular domain (codon optimized for Homosapiens)
  source 1..1416: mol_type = other DNA
ctgcctaaggctcccgtgatcaccacacctctggaaaccgtggatgccctggtggaagaggtggccacattcatgtgtgccgtggaaagctaccctcagcctgagatcagctggacccggaacaagatcctgatcaagctgttcgacacccggtacagcatcagagagaacggccagctgctgaccatcctgagcgtggaagatagcgacgacggcatctactgctgcaccgccaacaatggtgttggcggagccgtggaatcttgtggcgccctgcaagtgaagatgaagcccaagatcacccggcctccaatcaacgtgaagatcatcgagggcctgaaggccgtgctgccttgtaccacaatgggcaaccctaagcctagcgtgtcctggatcaagggcgacagccctctgagagagaacagcagaatcgccgtgctggaaagcggcagcctgaggatccacaacgtgcagaaagaggacgccggccagtacagatgcgtggccaagaattctctgggcaccgcctacagcaaggtggtcaagctggaagtggaagtgttcgcccggatcctgagagcccctgagtctcacaatgtgaccttcggcagcttcgtgaccctgcactgtacagccacaggcatccctgtgcctaccatcacctggatcgagaacggcaatgccgtgtccagcggcagcatccaagagtccgtgaaggacagagtgatcgacagcagactgcagctgttcatcaccaagcctggcctgtacacctgtatcgccaccaacaagcacggggagaagttctccacagccaaggccgctgccaccatctctattgccgagtggtccaagcctcagaaggacaacaagggctactgcgcccagtacaggggcgaagtgtgcaatgctgtgctggccaaagacgccctggtgttcctgaacacctcctacgccgatcctgaagaggcccaagaactgctggtgcacaccgcctggaatgagctgaaggtggtgtcccctgtgtgcagacctgctgctgaagccctgctgtgcaaccacatcttccaagagtgtagccctggcgtggtgcccacacctattcctatctgccgcgagtactgcctggccgtgaaagagctgttttgcgccaaagaatggctggtcatggaagagaaaacccaccggggcctgtatcggagcgaaatgcatctcctgtccgtgcctgagtgcagcaagctgcctagcatgcactgggaccctacagcctgtgccagactgccacacctggactacaacaaagagaacctcaagacgttccctccaatgaccagcagcaagcccagcgtggacatccccaatctgcctagctctagcagcagcagcttcagcgtgtcccctacctacagcatgacc

SEQ ID NO: 15 - protein, 89 aa, Homo sapiens
PVITTPLETVDALVEEVATFMCAVESYPQPEISWTRNKILIKLFDTRYSIRENGQLLTILSVEDSDDGIYCCTANNGVGGAVESCGALQ

SEQ ID NO: 16 - protein, 85 aa, Homo sapiens
PKITRPPINVKIIEGLKAVLPCTTMGNPKPSVSWIKGDSPLRENSRIAVLESGSLRIHNVQKEDAGQYRCVAKNSLGTAYSKVVK

SEQ ID NO: 17 - protein, 90 aa, Homo sapiens
RILRAPESHNVTFGSFVTLHCTATGIPVPTITWIENGNAVSSGSIQESVKDRVIDSRLQLFITKPGLYTCIATNKHGEKFSTAKAAATIS

SEQ ID NO: 18 - protein, 139 aa, Homo sapiens
DNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARL

SEQ ID NO: 19 - protein, 379 aa, synthetic construct
  REGION 1..379: note = Human MuSK ECD delta Ig-like 1
VKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMT

SEQ ID NO: 20 - DNA, 1137 bp, synthetic construct
  misc_feature 1..1137: note = Human MuSK ECD delta Ig-like 1
  source 1..1137: mol_type = other DNA
gtgaagatgaagcccaagatcacccggcctccaatcaacgtgaagatcatcgagggcctgaaggccgtgctgccttgtaccacaatgggcaaccctaagcctagcgtgtcctggatcaagggcgacagccctctgagagagaacagcagaatcgccgtgctggaaagcggcagcctgaggatccacaacgtgcagaaagaggacgccggccagtacagatgcgtggccaagaattctctgggcaccgcctacagcaaggtggtcaagctggaagtggaagtgttcgcccggatcctgagagcccctgagtctcacaatgtgaccttcggcagcttcgtgaccctgcactgtacagccacaggcatccctgtgcctaccatcacctggatcgagaacggcaatgccgtgtccagcggcagcatccaagagtccgtgaaggacagagtgatcgacagcagactgcagctgttcatcaccaagcctggcctgtacacctgtatcgccaccaacaagcacggggagaagttctccacagccaaggccgctgccaccatctctattgccgagtggtccaagcctcagaaggacaacaagggctactgcgcccagtacaggggcgaagtgtgcaatgctgtgctggccaaagacgccctggtgttcctgaacacctcctacgccgatcctgaagaggcccaagaactgctggtgcacaccgcctggaatgagctgaaggtggtgtcccctgtgtgcagacctgctgctgaagccctgctgtgcaaccacatcttccaagagtgtagccctggcgtggtgcccacacctattcctatctgccgcgagtactgcctggccgtgaaagagctgttttgcgccaaagaatggctggtcatggaagagaaaacccaccggggcctgtatcggagcgaaatgcatctcctgtccgtgcctgagtgcagcaagctgcctagcatgcactgggaccctacagcctgtgccagactgccacacctggactacaacaaagagaacctcaagacgttccctccaatgaccagcagcaagcccagcgtggacatccccaatctgcctagctctagcagcagcagcttcagcgtgtcccctacctacagcatgacc

SEQ ID NO: 21 - protein, 290 aa, synthetic construct
  REGION 1..290: note = Human MuSK no Ig-like 1 and 2 domain
LEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMT

SEQ ID NO: 22 - DNA, 870 bp, synthetic construct
  misc_feature 1..870: note = Human MuSK no Ig-like 1 and 2 domain
  source 1..870: mol_type = other DNA
ctggaagtggaagtgttcgcccggatcctgagagcccctgagtctcacaatgtgaccttcggcagcttcgtgaccctgcactgtacagccacaggcatccctgtgcctaccatcacctggatcgagaacggcaatgccgtgtccagcggcagcatccaagagtccgtgaaggacagagtgatcgacagcagactgcagctgttcatcaccaagcctggcctgtacacctgtatcgccaccaacaagcacggggagaagttctccacagccaaggccgctgccaccatctctattgccgagtggtccaagcctcagaaggacaacaagggctactgcgcccagtacaggggcgaagtgtgcaatgctgtgctggccaaagacgccctggtgttcctgaacacctcctacgccgatcctgaagaggcccaagaactgctggtgcacaccgcctggaatgagctgaaggtggtgtcccctgtgtgcagacctgctgctgaagccctgctgtgcaaccacatcttccaagagtgtagccctggcgtggtgcccacacctattcctatctgccgcgagtactgcctggccgtgaaagagctgttttgcgccaaagaatggctggtcatggaagagaaaacccaccggggcctgtatcggagcgaaatgcatctcctgtccgtgcctgagtgcagcaagctgcctagcatgcactgggaccctacagcctgtgccagactgccacacctggactacaacaaagagaacctcaagacgttccctccaatgaccagcagcaagcccagcgtggacatccccaatctgcctagctctagcagcagcagcttcagcgtgtcccctacctacagcatgacc

SEQ ID NO: 23 - protein, 193 aa, synthetic construct
  REGION 1..193: note = Human MuSK no Ig-like 1, 2 and 3 domain
IAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMT

SEQ ID NO: 24 - DNA, 579 bp, synthetic construct
  misc_feature 1..579: note = Human MuSK no Ig-like 1, 2 and 3 domain
  source 1..579: mol_type = other DNA
attgccgagtggtccaagcctcagaaggacaacaagggctactgcgcccagtacaggggcgaagtgtgcaatgctgtgctggccaaagacgccctggtgttcctgaacacctcctacgccgatcctgaagaggcccaagaactgctggtgcacaccgcctggaatgagctgaaggtggtgtcccctgtgtgcagacctgctgctgaagccctgctgtgcaaccacatcttccaagagtgtagccctggcgtggtgcccacacctattcctatctgccgcgagtactgcctggccgtgaaagagctgttttgcgccaaagaatggctggtcatggaagagaaaacccaccggggcctgtatcggagcgaaatgcatctcctgtccgtgcctgagtgcagcaagctgcctagcatgcactgggaccctacagcctgtgccagactgccacacctggactacaacaaagagaacctcaagacgttccctccaatgaccagcagcaagcccagcgtggacatccccaatctgcctagctctagcagcagcagcttcagcgtgtcccctacctacagcatgacc

SEQ ID NO: 25 - DNA, 363 bp, synthetic construct
  misc_feature 1..363: note = VH3D9b
  source 1..363: mol_type = other DNA
gaggtgcagctggtggaatctggcggcggacttgtgaaacctggcggctctctgagactgagctgtgccgcttccggcttcaccttcagcacctacacaatgcactgggtccgacaggcccctggcaaaggacttgaatgggtgtccagcatcagcagcagaagcgcctacaagtactacgccgacagcgtgaagggcagattcaccatcagccgggacaacgccaagaacagcctgtacctgcagatgaactccctgagagccgaggacaccgccgtgtactactgcgccagagacttctttcagctgggccctcctagattcgactcttggggacagggcaccctggtcacagttagctct

SEQ ID NO: 26 - DNA, 363 bp, synthetic construct
  misc_feature 1..363: note = VH3D10a
  source 1..363: mol_type = other DNA
gaggtgcagctggtggaatctggcggcggacttgtgaaacctggcggctctctgagactgagctgtgccgcttccggcttcacctttagcgcctctacaatggcctgggtccgacaggctcctggcaaaggacttgaatgggtgtccagcgtgtccggcgatagccaccacatctactacgccgacagcctgaagggcagattcaccatcagccgggacaacgccaagaacagcctgtacctgcagatgaactccctgagagccgaggacaccgccgtgtactattgcgccagagagagactgctgagactcggcgtgggctttgattcttggggccagggaacactggtcaccgtgtcatct

SEQ ID NO: 27 - DNA, 363 bp, synthetic construct
  misc_feature 1..363: note = VH3F6c
  source 1..363: mol_type = other DNA
gaggtgcagctggtggaatctggcggcggacttgtgaaacctggcggctctctgagactgagctgtgccgcttccggcttcaccttcagcagctacaccatggactgggtccgacaggcccctggcaaaggacttgaatgggtgtccagcatcggcagcaacggcgactacatctactacgccgatagcgtgcggggcagattcaccatctccagagacaacgccaagaacagcctgtacctgcagatgaactccctgagagccgaggacaccgccgtgtactattgtgccagaggacagctggccgtggccggcacacactttgattattggggccagggcaccctggtcaccgttagttct

SEQ ID NO: 28 - DNA, 363 bp, synthetic construct
  misc_feature 1..363: note = VH4D3
  source 1..363: mol_type = other DNA
gaggtgcagctggtggaatctggcggcggacttgtgaaacctggcggctctctgagactgagctgtgccgcttccggcttcaccttcagcagctacaccatgacctgggtccgacaggcccctggcaaaggacttgaatgggtgtccagcatcagctctggcggccactacatctactacaccgacagcctgaagggcagattcaccatctccagagacaacgccaagaacagcctgtacctgcagatgaactccctgagagccgaggacaccgccgtgtactattgcgccagagagagactgctgagactcggcgtgggcttcgatttttggggccagggaacactggtcaccgtgtctagt

SEQ ID NO: 29 - DNA, 363 bp, synthetic construct
  misc_feature 1..363: note = VH3B5
  source 1..363: mol_type = other DNA
caagtgcagctggtgcagtctggcgccgtggttgctaaacctggcgcctctgtgcaggtttcctgcaagacaagcggctacaccttcaccggccagtacctgcactgggttcgacaggctccaggacagggactcgagtggatcggctggatcaatcctagcagcggcgtgaccaagttcgccgagaagtttgagggcagagccaccatgaccagagacaccagcatcaccaccgcctacatcgacctgagaagcctgagatccgacgacaccgccacctactactgtgccacactgtctctgggcgtgtactacgttggaatggtggcctggggacagggcacactggttacagttagcagc

SEQ ID NO: 30 - DNA, 321 bp, synthetic construct
  misc_feature 1..321: note = VK3D9b
  source 1..321: mol_type = other DNA
gacatccagatgacacagagccccagcagcctgtctgcctctgtgggagacagagtgaccatcacctgtcgggccagccagagaatcagcagcttcctgaactggtatcagcagaagcccggcaaggcccctaaactgctgatctacggcgccagcaatctgcagagcggagtgcctagcagattttccggctctggcagcggcaccgatttcaccctgaccatatctagcctgcagcctgaggacttcgccacctactactgccagcagagctacagccccagctacacatttggccagggcaccaagctggaaatcaag

SEQ ID NO: 31 - DNA, 321 bp, synthetic construct
  misc_feature 1..321: note = VK3D10a
  source 1..321: mol_type = other DNA
gacatccagatgacacagagccccagcagcctgtctgcctctgtgggagacagagtgaccatcacctgtcgggccagccagagaatcagcggcttcgtgaactggtatcagcagaagcctggcaaggcccctaagctgctgatctacgccgctagcacactgcagagcggagtgcctagcagattttctggcagcggctccggcaccgatttcaccctgaccatatctagcctgcagcctgaggacttcgccacctactactgccagcagagctacagccctctgtacacctttggccagggcaccaagctggaaatcaag

SEQ ID NO: 32 - DNA, 321 bp, synthetic construct
  misc_feature 1..321: note = VK3F6c
  source 1..321: mol_type = other DNA
gacatccagatgacacagagccccagcagcctgtctgcctctgtgggagacagagtgaccatcacctgtcgggccagccagaaagtgaacaaatacgtgaactggtatcagcagaagcccggcaaggcccctaagctgctgatctatgccgccagcacactgcagagcggagtgccttctagattttccggcagcggctccggcaccgatttcaccctgaccatatctagcctgcagcctgaggacttcgccacctactactgccagcagagctacagccctctgagcacatttggccagggcaccaagctggaaatcaag

SEQ ID NO: 33 - DNA, 321 bp, synthetic construct
  misc_feature 1..321: note = VK4D3
  source 1..321: mol_type = other DNA
gacatccagatgacacagagccccagcagcctgtctgcctctgtgggagacagagtgaccatcacctgtagagccagccagagcatcagcggctacctgaactggtatcagcagaagcctggcaaggcccctaagctgctgatctacgccgctagcacactgcagagcggagtgcctagcagattttctggcagcggctccggcaccgatttcaccctgaccatatctagcctgcagcctgaggacttcgccacctactactgccagcagagctacagcgccctgtacacatttggccagggcaccaagctggaaatcaag

SEQ ID NO: 34 - DNA, 324 bp, synthetic construct
  misc_feature 1..324: note = VL3B5
  source 1..324: mol_type = other DNA
agctacgagctgacccagcctccaagcgtgtcagtgtctcctggacagaccgccaccatcacatgcagcggagatggactggctcagcagcacgtgtactggttccagcaaagacctggacaggcccctctgctgatcatctacaaggacatcgagcggcccagcggcatccctgagagattttctggcagcagcagcggcaccaccgccatgcttacaattagcggagtgcaggccgaggacgaggccgattactattgtcagagcggcgatagaaccgccacctccgttctttttggcggaggcaccaagatgaccgtgctg

SEQ ID NO: 35 - protein, 121 aa, synthetic construct
  REGION 1..121: note = VH3D9b
EVQLVESGGGLVKPGGSLRLSCAASGFTFSTYTMHWVRQAPGKGLEWVSSISSRSAYKYYADSVKGRFTISRDNAKNSLYLQMNSLRAEDTAVYYCARDFFQLGPPRFDSWGQGTLVTVSS

SEQ ID NO: 36 - protein, 121 aa, synthetic construct
  REGION 1..121: note = VH3D10a
EVQLVESGGGLVKPGGSLRLSCAASGFTFSASTMAWVRQAPGKGLEWVSSVSGDSHHIYYADSLKGRFTISRDNAKNSLYLQMNSLRAEDTAVYYCARERLLRLGVGFDSWGQGTLVTVSS

SEQ ID NO: 37 - protein, 121 aa, synthetic construct
  REGION 1..121: note = VH3F6c
EVQLVESGGGLVKPGGSLRLSCAASGFTFSSYTMDWVRQAPGKGLEWVSSIGSNGDYIYYADSVRGRFTISRDNAKNSLYLQMNSLRAEDTAVYYCARGQLAVAGTHFDYWGQGTLVTVSS

SEQ ID NO: 38 - protein, 121 aa, synthetic construct
  REGION 1..121: note = VH4D3
EVQLVESGGGLVKPGGSLRLSCAASGFTFSSYTMTWVRQAPGKGLEWVSSISSGGHYIYYTDSLKGRFTISRDNAKNSLYLQMNSLRAEDTAVYYCARERLLRLGVGFDFWGQGTLVTVSS

SEQ ID NO: 39 - protein, 121 aa, synthetic construct
  REGION 1..121: note = VH3B5
QVQLVQSGAVVAKPGASVQVSCKTSGYTFTGQYLHWVRQAPGQGLEWIGWINPSSGVTKFAEKFEGRATMTRDTSITTAYIDLRSLRSDDTATYYCATLSLGVYYVGMVAWGQGTLVTVSS

SEQ ID NO: 40 - protein, 5 aa, synthetic construct
  REGION 1..5: note = VH3D9b HCDR1
TYTMH

SEQ ID NO: 41 - protein, 5 aa, synthetic construct
  REGION 1..5: note = VH3D10a HCDR1
ASTMA

SEQ ID NO: 42 - protein, 5 aa, synthetic construct
  REGION 1..5: note = VH3F6c HCDR1
SYTMD

SEQ ID NO: 43 - protein, 5 aa, synthetic construct
  REGION 1..5: note = VH4D3 HCDR1
SYTMT

SEQ ID NO: 44 - protein, 5 aa, synthetic construct
  REGION 1..5: note = VH3B5 HCDR1
GQYLH

SEQ ID NO: 45 - protein, 17 aa, synthetic construct
  REGION 1..17: note = VH3D9b HCDR2
SISSRSAYKYYADSVKG

SEQ ID NO: 46 - protein, 17 aa, synthetic construct
  REGION 1..17: note = VH3D10a HCDR2
SVSGDSHHIYYADSLKG

SEQ ID NO: 47 - protein, 17 aa, synthetic construct
  REGION 1..17: note = VH3F6c HCDR2
SIGSNGDYIYYADSVRG

SEQ ID NO: 48 - protein, 17 aa, synthetic construct
  REGION 1..17: note = VH4D3 HCDR2
SISSGGHYIYYTDSLKG

SEQ ID NO: 49 - protein, 17 aa, synthetic construct
  REGION 1..17: note = VH3B5 HCDR2
WINPSSGVTKFAEKFEG

SEQ ID NO: 50 - protein, 12 aa, synthetic construct
  REGION 1..12: note = VH3D9b HCDR3
DFFQLGPPRFDS

SEQ ID NO: 51 - protein, 12 aa, synthetic construct
  REGION 1..12: note = VH3D10a HCDR3
ERLLRLGVGFDS

SEQ ID NO: 52 - protein, 12 aa, synthetic construct
  REGION 1..12: note = VH3F6c HCDR3
GQLAVAGTHFDY

SEQ ID NO: 53 - protein, 12 aa, synthetic construct
  REGION 1..12: note = VH4D3 HCDR3
ERLLRLGVGFDF

SEQ ID NO: 54 - protein, 12 aa, synthetic construct
  REGION 1..12: note = VH3B5 HCDR3
LSLGVYYVGMVA

SEQ ID NO: 55 - protein, 107 aa, synthetic construct
  REGION 1..107: note = VK3D9b
DIQMTQSPSSLSASVGDRVTITCRASQRISSFLNWYQQKPGKAPKLLIYGASNLQSGVPSRFSGSGSGTDFTLTISSLQPEDFATYYCQQSYSPSYTFGQGTKLEIK

SEQ ID NO: 56 - protein, 107 aa, synthetic construct
  REGION 1..107: note = VK3D10a
DIQMTQSPSSLSASVGDRVTITCRASQRISGFVNWYQQKPGKAPKLLIYAASTLQSGVPSRFSGSGSGTDFTLTISSLQPEDFATYYCQQSYSPLYTFGQGTKLEIK

SEQ ID NO: 57 - protein, 107 aa, synthetic construct
  REGION 1..107: note = VK3F6c
DIQMTQSPSSLSASVGDRVTITCRASQKVNKYVNWYQQKPGKAPKLLIYAASTLQSGVPSRFSGSGSGTDFTLTISSLQPEDFATYYCQQSYSPLSTFGQGTKLEIK

SEQ ID NO: 58 - protein, 107 aa, synthetic construct
  REGION 1..107: note = VK4D3
DIQMTQSPSSLSASVGDRVTITCRASQSISGYLNWYQQKPGKAPKLLIYAASTLQSGVPSRFSGSGSGTDFTLTISSLQPEDFATYYCQQSYSALYTFGQGTKLEIK

SEQ ID NO: 59 - protein, 106 aa, synthetic construct
  REGION 1..106: note = VL3B5
ELTQPPSVSVSPGQTATITCSGDGLAQQHVYWFQQRPGQAPLLIIYKDIERPSGIPERFSGSSSGTTAMLTISGVQAEDEADYYCQSGDRTATSVLFGGGTKMTVL

SEQ ID NO: 60 - protein, 11 aa, synthetic construct
  REGION 1..11: note = VK3D9b LCDR1
RASQRISSFLN

SEQ ID NO: 61 - protein, 11 aa, synthetic construct
  REGION 1..11: note = VK3D10a LCDR1
RASQRISGFVN

SEQ ID NO: 62 - protein, 11 aa, synthetic construct
  REGION 1..11: note = VK3F6c LCDR1
RASQKVNKYVN

SEQ ID NO: 63 - protein, 11 aa, synthetic construct
  REGION 1..11: note = VK4D3 LCDR1
RASQSISGYLN

SEQ ID NO: 64 - protein, 11 aa, synthetic construct
  REGION 1..11: note = VL3B5 LCDR1
SGDGLAQQHVY

SEQ ID NO: 65 - protein, 7 aa, synthetic construct
  REGION 1..7: note = VK3D9b LCDR2
GASNLQS

SEQ ID NO: 66 - protein, 7 aa, synthetic construct
  REGION 1..7: note = VK3D10a LCDR2
AASTLQS

SEQ ID NO: 67 - protein, 7 aa, synthetic construct
  REGION 1..7: note = VK3F6c LCDR2
AASTLQS

SEQ ID NO: 68 - protein, 7 aa, synthetic construct
  REGION 1..7: note = VK4D3 LCDR2
AASTLQS

SEQ ID NO: 69 - protein, 7 aa, synthetic construct
  REGION 1..7: note = VL3B5 LCDR2
KDIERPS

SEQ ID NO: 70 - protein, 9 aa, synthetic construct
  REGION 1..9: note = VK3D9b LCDR3
QQSYSPSYT

SEQ ID NO: 71 - protein, 9 aa, synthetic construct
  REGION 1..9: note = VK3D10a LCDR3
QQSYSPLYT

SEQ ID NO: 72 - protein, 9 aa, synthetic construct
  REGION 1..9: note = VK3F6c LCDR3
QQSYSPLST

SEQ ID NO: 73 - protein, 9 aa, synthetic construct
  REGION 1..9: note = VK4D3 LCDR3
QQSYSALYT

SEQ ID NO: 74 - protein, 11 aa, synthetic construct
  REGION 1..11: note = VL3B5 LCDR3
QSGDRTATSVL

SEQ ID NO: 75 - DNA, 987 bp, synthetic construct
  misc_feature 1..987: note = hIgG1LALAdelK
  source 1..987: mol_type = other DNA
gccagtacaaaaggtccaagtgtgttccctcttgctccctcatccaagagtaccagtggaggcaccgccgctcttggctgcttggttaaggattatttcccagagcctgtcactgtttcatggaactccggcgccttgacatctggtgtgcatacctttccagccgtgctgcagtcaagtggcctctacagcctcagtagcgtggtcactgtgcccagcagctctctcggcacacaaacttatatctgtaatgtgaatcataagccttcaaataccaaggtggataagaaagtggaaccaaaatcatgtgacaagacacacacctgccctccttgtccagcccccgaagcggccggtgggcccagcgtgttcctgtttcctcctaaacccaaagacactctgatgattagtaggaccccagaagtcacttgcgtggtggttgacgtgtcacatgaagatcccgaggtcaagttcaattggtatgttgacggggtcgaagttcacaacgctaaaactaaaccaagagaggaacagtataactctacctaccgggtggtgagtgttctgactgtcctccatcaagactggctgaatggcaaagaatacaagtgtaaggtgagcaacaaagccctgcccgctcctatagagaaaacaatatccaaagccaaaggtcaacctcgcgagccacaggtgtacaccctcccaccaagccgcgatgaacttactaagaaccaagtctctcttacttgcctggttaaggggttctatccatccgacattgcagtcgagtgggagtctaatggacagcctgagaacaactacaaaaccacccctcctgttctggattctgacggatctttcttcctttattctaaactcaccgtggataaaagcaggtggcagcagggcaacgtgttcagctgttccgttatgcatgaggccctgcataaccattatacccagaagtctttgtccctcagtccagga

SEQ ID NO: 76 - protein, 329 aa, synthetic construct
  REGION 1..329: note = hIgG1LALAdelK
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - DNA, 981 bp, synthetic construct
  misc_feature 1..981: note = hIgG4_S228P
  source 1..981: mol_type = other DNA
gctagcaccaagggcccctccgtgttccccctggccccttgctcccggtccacctccgagtctaccgccgctctgggctgcctggtgaaagactacttccccgagcctgtgaccgtgagctggaactctggcgccctgacctccggcgtgcacaccttccctgccgtgctgcaatcctccggcctgtactccctgtcctccgtggtgacagtgccctcctccagcctgggcaccaagacctacacctgtaacgtggaccacaagccctccaacaccaaggtggacaagcgggtggaatctaaatacggccctccctgccccccctgccctgcccctgaatttctgggcggaccttccgtgtttctgttccccccaaagcccaaggacaccctgatgatctcccggacccccgaagtgacctgcgtggtggtggacgtgtcccaggaagatccagaggtgcagttcaactggtatgttgacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtgaaaggcttctacccctccgacattgccgtggaatgggagtccaacggccagcccgagaacaactacaagaccaccccccctgtgctggactccgacggctccttcttcctgtactctcggctgacagtggataagtcccggtggcaggaaggcaacgtgttctcctgcagcgtgatgcacgaggccctgcacaaccactatacccagaagtccctgtccctgagcctgggcaag

SEQ ID NO: 78 - protein, 327 aa, synthetic construct
  REGION 1..327: note = hIgG4_S228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 869 aa, Macaca fascicularis
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRTAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPLCRPAAEALLCNHVFQECSPGVVPTPIPICREYCLAVKELFCAKEWLAMEEKTHRGLYRSEMHLLSVPECSKLPSMHWDPTACARLPHLDYNKENPKTFPPMTSSKPSVDIPNLPSSSSSSFSVSPTYSMTVIISIMSSFAIFALLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 80 - protein, 472 aa, Macaca fascicularis
LPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRTAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPLCRPAAEALLCNHVFQECSPGVVPTPIPICREYCLAVKELFCAKEWLAMEEKTHRGLYRSEMHLLSVPECSKLPSMHWDPTACARLPHLDYNKENPKTFPPMTSSKPSVDIPNLPSSSSSSFSVSPTYSMT

SEQ ID NO: 81 - protein, 869 aa, Macaca mulatta
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRTAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATVSIAEWSKPQKDNKGYCAQYRGEVCNAVLAKDALVFLNTSYADPEEAQELLVHTAWNELKVVSPLCRPAAEALLCNHVFQECSPGVVPTPIPICREYCLAVKELFCAKEWLAMEEKTHRGLYRSEMHLLSVPECSKLPSMHWDPTACARLPHLDYNKENPKTFPPMTSSKPSVDIPNLPSSSSSSFSVSPTYSMTVIISIMSSFAIFALLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 82 - protein, 472 aa, Macaca mulatta
LPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRTAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATVSIAEWSKPQKDNKGYCAQYRGEVCNAVLAKDALVFLNTSYADPEEAQELLVHTAWNELKVVSPLCRPAAEALLCNHVFQECSPGVVPTPIPICREYCLAVKELFCAKEWLAMEEKTHRGLYRSEMHLLSVPECSKLPSMHWDPTACARLPHLDYNKENPKTFPPMTSSKPSVDIPNLPSSSSSSFSVSPTYSMT

SEQ ID NO: 83 - protein, 473 aa, Mus musculus
EKLPKAPVITTPLETVDALVEEVATFMCAVESYPQPEISWTRNKILIKLFDTRYSIRENGQLLTILSVEDSDDGIYCCIANNGVGGAVESCGALQVKMKPKITRPPINVKIIEGLKAVLPCTTMGNPKPSVSWIKGDNALRENSRIAVLESGSLRIHNVQKEDAGQYRCVAKNSLGTAYSKLVKLEVEVFARILRAPESHNVTFGSFVTLRCTAIGIPVPTISWIENGNAVSSGSIQESVKDRVIDSRLQLFITKPGLYTCIATNKHGEKFSTAKAAATVSIAEWSKSQKDSQGYCAQYRGEVCDAVLAKDALVFFNTSYRDPEDAQELLIHTAWNELKAVSPLCRPAAEALLCNHLFQECSPGVVPTPMPICREYCLAVKELFCAKEWQAMEGKAHRGLYRSGMHLLPVPECSKLPSMHRDPTACTRLPYLDYKKENITTFPSITSSRPSADIPNLPASTSSFAVSPAYSMT

SEQ ID NO: 84 - protein, 473 aa, Rattus norvegicus
EKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSALRENSRIAVLESGSLRIHNVQKEDAGQYRCVAKNSLGTAYSKLVKLEVEVFARILRAPESHNVTFGSFVTLRCTAIGMPVPTISWIENGNAVSSGSIQENVKDRVIDSRLQLFITKPGLYTCIATNKHGEKFSTAKAAATVSIAEWSKSQKESKGYCAQYRGEVCDAVLVKDSLVFFNTSYPDPEEAQELLIHTAWNELKAVSPLCRPAAEALLCNHLFQECSPGVLPTPMPICREYCLAVKELFCAKEWLAMEGKTHRGLYRSGMHFLPVPECSKLPSMHQDPTACTRLPYLDYKKENITTFPSITSSKPSVDIPNLPASTSSFAVSPAYSMT

SEQ ID NO: 85 - protein, 30 aa, synthetic construct
  REGION 1..30: note = VH3D9b FR1
EVQLVESGGGLVKPGGSLRLSCAASGFTFS

SEQ ID NO: 86 - protein, 30 aa, synthetic construct; identical to SEQ ID NO: 85
  REGION 1..30: note = VH3D10a FR1

SEQ ID NO: 87 - protein, 30 aa, synthetic construct; identical to SEQ ID NO: 85
  REGION 1..30: note = VH3F6c FR1

SEQ ID NO: 88 - protein, 30 aa, synthetic construct; identical to SEQ ID NO: 85
  REGION 1..30: note = VH4D3 FR1

SEQ ID NO: 89 - protein, 30 aa, synthetic construct
  REGION 1..30: note = VH3B5 FR1
QVQLVQSGAVVAKPGASVQVSCKTSGYTFT

SEQ ID NO: 90 - protein, 14 aa, synthetic construct
  REGION 1..14: note = VH3D9b FR2
WVRQAPGKGLEWVS

SEQ ID NO: 91 - protein, 14 aa, synthetic construct
  REGION 1..14: note = VH3D10a FR2
WVRQAPGKGLEWVS

SEQ ID NO: 92 - protein, 14 aa, synthetic construct
  REGION 1..14: note = VH3F6c FR2
WVRQAPGKGLEWVS

SEQ ID NO: 93 - protein, 14 aa, synthetic construct
  REGION 1..14: note = VH4D3 FR2
WVRQAPGKGLEWVS

SEQ ID NO: 94 - protein, 14 aa, synthetic construct
  REGION 1..14: note = VH3B5 FR2
WVRQAPGQGLEWIG

SEQ ID NO: 95 - protein, 32 aa, synthetic construct
  REGION 1..32: note = VH3D9b FR3
RFTISRDNAKNSLYLQMNSLRAEDTAVYYCAR

SEQ ID NO: 96 - protein, 32 aa, synthetic construct; identical to SEQ ID NO: 95
  REGION 1..32: note = VH3D10a FR3

SEQ ID NO: 97 - protein, 32 aa, synthetic construct; identical to SEQ ID NO: 95
  REGION 1..32: note = VH3F6c FR3

SEQ ID NO: 98 - protein, 32 aa, synthetic construct; identical to SEQ ID NO: 95
  REGION 1..32: note = VH4D3 FR3

SEQ ID NO: 99 - protein, 32 aa, synthetic construct
  REGION 1..32: note = VH3B5 FR3
RATMTRDTSITTAYIDLRSLRSDDTATYYCAT

SEQ ID NO: 100 - protein, 11 aa, synthetic construct
  REGION 1..11: note = VH3D9b FR4
WGQGTLVTVSS

SEQ ID NO: 101 - protein, 11 aa, synthetic construct
  REGION 1..11: note = VH3D10a FR4
WGQGTLVTVSS

SEQ ID NO: 102 - protein, 11 aa, synthetic construct
  REGION 1..11: note = VH3F6c FR4
WGQGTLVTVSS

SEQ ID NO: 103 - protein, 11 aa, synthetic construct
  REGION 1..11: note = VH4D3 FR4
WGQGTLVTVSS

SEQ ID NO: 104 - protein, 11 aa, synthetic construct
  REGION 1..11: note = VH3B5 FR4
WGQGTLVTVSS

SEQ ID NO: 105 - protein, 23 aa, synthetic construct
  REGION 1..23: note = VL3D9b FR1
DIQMTQSPSSLSASVGDRVTITC

SEQ ID NO: 106 - protein, 23 aa, synthetic construct
  REGION 1..23: note = VL3D10a FR1
DIQMTQSPSSLSASVGDRVTITC

SEQ ID NO: 107 - protein, 23 aa, synthetic construct
  REGION 1..23: note = VL3F6c FR1
DIQMTQSPSSLSASVGDRVTITC

SEQ ID NO: 108 - protein, 23 aa, synthetic construct
  REGION 1..23: note = VL4D3 FR1
DIQMTQSPSSLSASVGDRVTITC

SEQ ID NO: 109 - protein, 22 aa, synthetic construct
  REGION 1..22: note = VL3B5 FR1
SYELTQPPSVSVSPGQTATITC

SEQ ID NO: 110 - protein, 15 aa, synthetic construct
  REGION 1..15: note = VL3D9b FR2
WYQQKPGKAPKLLIY

SEQ ID NO: 111 - protein, 15 aa, synthetic construct
  REGION 1..15: note = VL3D10a FR2
WYQQKPGKAPKLLIY

SEQ ID NO: 112 - protein, 15 aa, synthetic construct
  REGION 1..15: note = VL3F6c FR2
WYQQKPGKAPKLLIY

SEQ ID NO: 113 - protein, 15 aa, synthetic construct
  REGION 1..15: note = VL4D3 FR2
WYQQKPGKAPKLLIY

SEQ ID NO: 114 - protein, 15 aa, synthetic construct
  REGION 1..15: note = VL3B5 FR2
WFQQRPGQAPLLIIY

SEQ ID NO: 115 - protein, 32 aa, synthetic construct
  REGION 1..32: note = VL3D9b FR3
GVPSRFSGSGSGTDFTLTISSLQPEDFATYYC

SEQ ID NO: 116 - protein, 32 aa, synthetic construct; identical to SEQ ID NO: 115
  REGION 1..32: note = VL3D10a FR3

SEQ ID NO: 117 - protein, 32 aa, synthetic construct; identical to SEQ ID NO: 115
  REGION 1..32: note = VL3F6c FR3

SEQ ID NO: 118 - protein, 32 aa, synthetic construct; identical to SEQ ID NO: 115
  REGION 1..32: note = VL4D3 FR3

SEQ ID NO: 119 - protein, 32 aa, synthetic construct
  REGION 1..32: note = VL3B5 FR3
GIPERFSGSSSGTTAMLTISGVQAEDEADYYC

SEQ ID NO: 120 - protein, 10 aa, synthetic construct
  REGION 1..10: note = VL3D9b FR4
FGQGTKLEIK

SEQ ID NO: 121 - protein, 10 aa, synthetic construct
  REGION 1..10: note = VL3D10a FR4
FGQGTKLEIK

SEQ ID NO: 122 - protein, 10 aa, synthetic construct
  REGION 1..10: note = VL3F6c FR4
FGQGTKLEIK

SEQ ID NO: 123 - protein, 10 aa, synthetic construct
  REGION 1..10: note = VL4D3 FR4
FGQGTKLEIK

SEQ ID NO: 124 - protein, 10 aa, synthetic construct
  REGION 1..10: note = VL3B5 FR4
FGGGTKMTVL

SEQ ID NO: 125 - protein, 448 aa, synthetic construct
  REGION 1..448: note = 3B5 hIgG4_S228P heavy chain
QVQLVQSGAVVAKPGASVQVSCKTSGYTFTGQYLHWVRQAPGQGLEWIGWINPSSGVTKFAEKFEGRATMTRDTSITTAYIDLRSLRSDDTATYYCATLSLGVYYVGMVAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 126 - protein, 212 aa, synthetic construct
  REGION 1..212: note = 3B5 hIgG4_S228P light chain
ELTQPPSVSVSPGQTATITCSGDGLAQQHVYWFQQRPGQAPLLIIYKDIERPSGIPERFSGSSSGTTAMLTISGVQAEDEADYYCQSGDRTATSVLFGGGTKMTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 127 - protein, 450 aa, synthetic construct
  REGION 1..450: note = 3D10a hIgG1LALAdelK heavy chain
EVQLVESGGGLVKPGGSLRLSCAASGFTFSASTMAWVRQAPGKGLEWVSSVSGDSHHIYYADSLKGRFTISRDNAKNSLYLQMNSLRAEDTAVYYCARERLLRLGVGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 214 aa, synthetic construct
  REGION 1..214: note = 3D10a hIgG1LALAdelK light chain
DIQMTQSPSSLSASVGDRVTITCRASQRISGFVNWYQQKPGKAPKLLIYAASTLQSGVPSRFSGSGSGTDFTLTISSLQPEDFATYYCQQSYSPLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 450 aa, synthetic construct
  REGION 1..450: note = 4D3 hIgG1LALAdelK heavy chain
EVQLVESGGGLVKPGGSLRLSCAASGFTFSSYTMTWVRQAPGKGLEWVSSISSGGHYIYYTDSLKGRFTISRDNAKNSLYLQMNSLRAEDTAVYYCARERLLRLGVGFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 214 aa, synthetic construct
  REGION 1..214: note = 4D3 hIgG1LALAdelK light chain
DIQMTQSPSSLSASVGDRVTITCRASQSISGYLNWYQQKPGKAPKLLIYAASTLQSGVPSRFSGSGSGTDFTLTISSLQPEDFATYYCQQSYSALYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 450 aa, synthetic construct
  REGION 1..450: note = 3F6c hIgG1LALAdelK heavy chain
EVQLVESGGGLVKPGGSLRLSCAASGFTFSSYTMDWVRQAPGKGLEWVSSIGSNGDYIYYADSVRGRFTISRDNAKNSLYLQMNSLRAEDTAVYYCARGQLAVAGTHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 214 aa, synthetic construct
  REGION 1..214: note = 3F6c hIgG1LALAdelK light chain
DIQMTQSPSSLSASVGDRVTITCRASQKVNKYVNWYQQKPGKAPKLLIYAASTLQSGVPSRFSGSGSGTDFTLTISSLQPEDFATYYCQQSYSPLSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 450 aa, synthetic construct
  REGION 1..450: note = 3D9b hIgG1LALAdelK heavy chain
EVQLVESGGGLVKPGGSLRLSCAASGFTFSTYTMHWVRQAPGKGLEWVSSISSRSAYKYYADSVKGRFTISRDNAKNSLYLQMNSLRAEDTAVYYCARDFFQLGPPRFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 214 aa, synthetic construct
  REGION 1..214: note = 3D9b hIgG1LALAdelK light chain
DIQMTQSPSSLSASVGDRVTITCRASQRISSFLNWYQQKPGKAPKLLIYGASNLQSGVPSRFSGSGSGTDFTLTISSLQPEDFATYYCQQSYSPSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC